SEQUENCE LISTING (6 sequences)

SEQ ID NO: 1 - DNA, 462 bp, synthetic construct
  misc_feature 1..462: note = synthetic oligonucleotide
  source 1..462: mol_type = other DNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 2 - protein, 153 aa, synthetic construct
  REGION 1..153: note = synthetic oligonucleotide
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 3 - DNA, 462 bp, synthetic construct
  misc_feature 1..462: note = synthetic oligonucleotide
  source 1..462: mol_type = other DNA
atgtaccggatgcagctgctgtcctgcatcgcactgtccctcgccctggtgacaaattctgcccccacctcctccagcacaaaaaagacccagttgcagctggagcacctgctgctggatctgcagatgatcctgaatggcatcaataactacaaaaaccctaaactgaccagaatgctgacctttaaattttacatgcctaaaaaggcaaccgagctgaagcacctgcagtgcctggaagaggaactgaagcccctggaggaggtgctgaacctggcccagagcaagaactttcacctgcggccccgcgacctgatcagcaacatcaacgtgatcgtgctggagctgaagggcagtgaaaccacattcatgtgcgagtacgccgacgagaccgccacaatcgtggagttcctgaacagatggatcacattctgtcagtccatcattagcacactgacctaa

SEQ ID NO: 4 - DNA, 462 bp, synthetic construct
  misc_feature 1..462: note = synthetic oligonucleotide
  source 1..462: mol_type = other DNA
atgtaccgcatgcagctgctgagctgcatcgccctgagcctggccctggtgaccaacagcgcccccaccagcagcagcaccaagaagacccagctgcagctggagcacctgctgctggacctgcagatgatcctgaacggcatcaacaactacaagaaccccaagctgacccgcatgctgaccttcaagttctacatgcccaagaaggccaccgagctgaagcacctgcagtgcctggaggaggagctgaagcccctggaggaggtgctgaacctggcccagagcaagaacttccacctgcgcccccgcgacctgatcagcaacatcaacgtgatcgtgctggagctgaagggcagcgagaccaccttcatgtgcgagtacgccgacgagaccgccaccatcgtggagttcctgaaccgctggatcaccttctgccagagcatcatcagcaccctgacctaa

SEQ ID NO: 5 - DNA, 459 bp, synthetic construct
  misc_feature 1..459: note = synthetic oligonucleotide
  source 1..459: mol_type = other DNA
atgtataggatgcagctgctctcttgtatcgcgttgtctctggctttggtgactaactcagctcccacgtccagcagtaccaaaaagacccagctgcagctggaacatcttctgttggatctgcaaatgatactgaatgggatcaacaactataaaaacccaaaactgactagaatgctgactttcaagttctacatgcctaaaaaggcaacagaattgaagcaccttcagtgcctggaggaggagcttaagcccctggaggaggtgctgaatctggcccaaagtaagaattttcatctgcgacccagggatctgatcagtaatatcaatgtgatcgtcctggagctgaagggcagtgagaccacgtttatgtgtgaatacgcagacgaaaccgccactatcgttgaattcttgaacaggtggatcaccttttgtcagagtatcatcagcaccctcact

SEQ ID NO: 6 - DNA, 462 bp, synthetic construct
  misc_feature 1..462: note = synthetic oligonucleotide
  source 1..462: mol_type = other DNA
atgtacagaatgcagctgctgagctgcatcgccctgagcctggccctggtgaccaacagcgcccccacaagcagcagcaccaagaagacacagctgcagctggagcacctgctgctggacctgcagatgatcctgaacggcatcaacaactacaagaaccccaagctgacaagaatgctgaccttcaagttctacatgcccaagaaggccaccgagctgaagcacctgcagtgcctggaggaggagctgaagcccctggaagaggtgctgaacctggctcagagcaagaacttccacctgagacctagagacctgatcagcaacatcaacgtgatcgtgctggagctgaagggcagcgagaccaccttcatgtgcgagtacgccgacgagaccgccaccatcgtggagttcctgaacagatggatcaccttctgtcagagcatcatcagcaccctgacctga

(19) United States
(12) Patent Application Publication
(10) Pub. No.: US 2024/0415931 A1
(43) Pub. Date: Dec. 19, 2024

(54) METHODS OF USE AND ADMINISTRATION OF ENCAPSULATED CELLS

(71) Applicant: William Marsh Rice University, Houston, TX (US)

(72) Inventors: Omid VEISEH, Houston, TX (US); Amanda NASH, Houston, TX (US); Samira AGHLARA-FOTOVAT, Houston, TX (US)

(73) Assignee: William Marsh Rice University, Houston, TX (US)

(21) Appl. No.: 18/703,201

(22) PCT Filed: Oct. 19, 2022
(86) PCT No.: PCT/US2022/078381

§ 371 Date: Apr. 19, 2024

Related U.S. Application Data
(60) Provisional application No. 63/342,212, filed on May 16, 2022.
(60) Provisional application No. 63/257,899, filed on Oct. 20, 2021.

(51) Int. Cl. A61K 38/20; A61K 9/50; A61K 35/12; A61P 35/00

(52) U.S. Cl. CPC A61K 38/2013; A61K 9/5036; A61K 9/5068; A61P 35/00; A61K 2035/128

(57) ABSTRACT

A fundamental barrier to successful device-based therapies is the Inability to deliver a sustained amount of therapeutics that do not have a systemic toxic impact on the subject. Thus, there is a need for identifying new compositions and methods to enhance the delivery, distribution, and/or efficacy of therapeutic agents. The present disclosure relates to implantable constructs (encapsulated cells) designed to deliver antigenic therapeutic reagents, such as IL-2, and optionally one additional therapeutic.

### CROSS-REFERENCE TO RELATED APPLICATIONS

This application claims priority to U.S. Provisional Application No. 63/257,899, filed Oct. 20, 2021, and U.S. Provisional Application No. 63/342,212, filed May 16, 2022, each of which is hereby incorporated by reference in its entirety.

### STATEMENT REGARDING FEDERALLY FUNDED RESEARCH

This invention was made with government support under Grant No. R01DK120459, awarded by the National Institutes of Health. The government has certain rights in the invention.

### REFERENCE TO A SEQUENCE LISTING

This application contains a Sequence Listing XML, which has been submitted electronically and is hereby incorporated by reference in its entirety. Said XML Sequence Listing, created on Oct. 19, 2022, is named RICEP0096WO.xml and is 11,033 bytes in size.

### BACKGROUND

### I. Field

The present disclosure relates to the fields of biology, medicine, bioengineering and medicals devices. More particular, it relates to the development and use of implantable constructs designed to deliver antigenic therapeutic reagents to a subject and protect them from immune responses generated by the host. In particular, the constructs are designed to degrade over time or upon a particular signal, thereby providing control of the length of time the therapeutic agent is delivered to the subject.

### II. Related Art

Advances in biomedical research have led to methods for localized and targeted therapies for the treatment of diseases, such as cancer. However, in many instances, the percentage of patients responsive to these approaches remain modest (Park et al., Sci. Transl. Med. 10(433) 2018).

One approach involves the use of implantable devices to deliver therapeutic agents, however, a fundamental barrier to successful device-based therapies is the inability to deliver a sustained amount of therapeutics that do not have a systemic toxic impact on the subject. Thus, there is a need for identifying new compositions and methods to enhance the delivery, distribution, and/or efficacy of therapeutic agents.

The development of this invention was funded in part by the Cancer Prevention and Research Institute of Texas under Grant No. RR160047.

### SUMMARY

Thus, in accordance with the present disclosure, there is provided. a method of delivering a native IL-2 to the pleural cavity of subject, the method comprising implanting, or delivering to, the pleural cavity a pharmaceutical composition comprising a population of encapsulated cells comprising a heterologous oligonucleotide molecule encoding the native human IL-2. Also provided is a method of treating a disease, in a subject, the method comprising implanting, or delivering to, the pleural cavity of the subject a pharmaceutical composition comprising a population of encapsulated cells comprising a heterologous oligonucleotide molecule encoding the native human IL-2. Also provided is a method of treating a pleural disease or condition, in a subject, the method comprising implanting, or delivering to, the pleural cavity a pharmaceutical composition comprising a population of encapsulated cells comprising a heterologous oligonucleotide molecule encoding the native human IL-2. Also provided is a method of treating a pleural disease or condition, in a subject by generating memory immunity, the method comprising implanting, or delivering to, the pleural cavity a pharmaceutical composition comprising a population of encapsulated cells comprising a heterologous oligonucleotide molecule encoding the native human IL-2.

The pleural disease or condition may be pleural cancer, pleural metastatic disease, pleurisy, lung infection, viral pneumonia, bacterial pneumonia, idiopathic pulmonary fibrosis, acute respiratory distress syndrome, pleural thickening, pleural pseudotumor, pleural plaque, extrapleural hematoma, Castleman disease, hemangioendothelioma, splenosis, paramalignang effusion, pleural effusion, pneumothorax, hemothorax, reactive pleuritis. The pleural cancer may be lung cancer, metastases, mesothelioma, malignant mesothelioma, lymphoma, malignant fibrous tumor, sarcoma, askin tumor, extraskeletal osteosarcoma, malignant fibrous histiocytoma, solitary fibrous tumor, lipoma, mesothelial cyst, calcifying fibrous pseudotumor, primary effusion lymphoma.

In another embodiment, there is provided a method of treating a mesothelioma, in a subject, the method comprising implanting, or delivering to, the pleural cavity a pharmaceutical composition comprising a population of encapsulated cells comprising a heterologous oligonucleotide molecule encoding the native human IL-2. The mesothelioma may be a pleural mesothelioma, a malignant pleural mesothelioma, or a diffuse pleural mesothelioma. Also provided is a method of treating a mesothelioma, in a subject by generating memory immunity, the method comprising implanting, or delivering to, the pleural cavity a pharmaceutical composition comprising a population of encapsulated cells comprising a heterologous oligonucleotide molecule encoding the native human IL-2. The mesothelioma may be a pleural mesothelioma, a malignant pleural mesothelioma, or a diffuse pleural mesothelioma.

In yet another embodiment, there is provided a method of providing systemic treatment to a subject with cancer, the method comprising implanting in the pleural cavity of the subject a pharmaceutical composition comprising a population of encapsulated cells comprising a heterologous oligonucleotide molecule encoding the native human IL-2, whereby the pharmaceutical composition stimulates the activation of immune cells in the pleural cavity and the activated immune cells migrate to a region of the subject that is distal to the pleural cavity to treat the cancer systemically in the subject. Also provided is a method of providing systemic treatment to a subject with cancer, the method comprising implanting in the pleural cavity of the subject a pharmaceutical composition comprising a population of encapsulated cells comprising a heterologous oligonucleotide molecule encoding the native human IL-2, whereby the pharmaceutical composition activates immune cells and the activated immune cells migrate out of the pleural cavity to treat the cancer in the subject.

The subject may have fewer side effects as compared to a subject that is administered the pharmaceutical composition systemically, such as intravenously. The activated immune cells may be CD8 positive effector T cells. The effector T cells may be selectively activated and expanded at least 1, 2, 3, 4, or 5 times as compared to Tregs in the pleural cavity. The effector T cells may be selectively activated and expanded at least 1, 2, 3, 4, or 5 times as compared to Tregs systemically. The subject may be administered about 0.01 μg/kg/day to about 20 μg/kg/day, about 0.1 μg/kg/day to about 20 μg/kg/day, about 1 μg/kg/day to about 20 μg/kg/day, about 2 μg/kg/day to about 20 μg/kg/day, about 5 μg/kg/day to about 20 μg/kg/day, about 7.5 to about 20 μg/kg/day, about 9 μg/kg/day to about 20 μg/kg/day, about 10 μg/kg/day to about 20 μg/kg/day, about 11 μg/kg/day to about 20 μg/kg/day, about 12 μg/kg/day to about 20 μg/kg/day, about 13 μg/kg/day to about 20 μg/kg/day, about 14 μg/kg/day to about 15 μg/kg/day, about 15 μg/kg/day to about 20 μg/kg/day, about 10 μg/kg/day to about 15 μg/kg/day, about 11 μg/kg/day to about 15 μg/kg/day, about 12 μg/kg/day to about 15 μg/kg/day, about 13 μg/kg/day to about 15 μg/kg/day, about 14 μg/kg/day to about 15 μg/kg/day, about 16 μg/kg/day to about 20 μg/kg/day, about 17 μg/kg/day to about 20 μg/kg/day, about 18 μg/kg/day to about 20 μg/kg/day, about 0.01 μg/kg/day, about 0.1 μg/kg/day, about 1 μg/kg/day, about 2 μg/kg/day, about 3 μg/kg/day, about 4 μg/kg/day, about 5 μg/kg/day, about 6 μg/kg/day, about 7 μg/kg/day, about 8 μg/kg/day, about 9 μg/kg/day, about 10 μg/kg/day, about 11 μg/kg/day, about 12 μg/kg/day, about 13 μg/kg/day, about 14 μg/kg/day, about 15 μg/kg/day, about 6 μg/kg/day, about 17 μg/kg/day, about 18 μg/kg/day, about 19 μg/kg/day, or about 20 μg/kg/day, of the encapsulated cells. The concentration of native human IL-2 in the pleural fluid at day 1 post implantation may be at least 3000 μg/ml, 4000 μg/ml, 5000 μg/ml, 10000 μg/ml, 15000 μg/ml, or 20000 μg/ml. The concentration of the recombinant native human IL-2 in the blood of the subject may be substantially undetectable 1 day after implantation. The concentration of the recombinant native human IL-2 in the pleural fluid of the subject may be substantially undetectable 30 days after implantation. The concentration of the recombinant native human IL-2 in the blood of the subject may be substantially undetectable 1 day after implantation and is at least 3000 μg/ml, 4000 μg/ml, 5000 μg/ml, 10000 μg/ml, 15000 μg/ml, or 20000 μg/ml in the pleural fluid of the subject. The pharmaceutical composition may be implanted according to a method or using a device as provided for herein.

As used herein, the term “recombinant native human IL-2 protein” or “native human IL-2 protein” refers to a protein that comprises the post-translational modifications of IL-2 produced by a cell, such as a eukaryotic cell (e.g., human) expressing endogenous IL-2, wherein the IL-2 is encoded for by a heterologous nucleic acid molecule that is added to the cell through manipulation (e.g., transduction, transformation, transfection, electroporation, and the like). For example, the heterologous IL-2 produced by the encapsulated cells provided for herein when compared to wild-type IL-2 produced by a cell in the subject has the same or similar post-translational modifications.

The oligonucleotide encoding native human IL-2 may comprise a sequence of: ATGTACAGGATGCAACTCCTGTCTTGCATTGCACTAAGTCTTGCACTTGTCACAA ACAGTGCACCTACTTCAAGTTCTACAAAGAAAACACAGCTACAACTGGAGCATT TACTGCTGGATTTACAGATGATTTTGAATGGAATTAATAATTACAAGAATCCCAA ACTCACCAGGATGCTCACATTTAAGTTTTACATGCCCAAGAAGGCCACAGAACTG AAACATCTTCAGTGTCTAGAAGAAGAACTCAAACCTCTGGAGGAAGTGCTAAAT TTAGCTCAAAGCAAAAACTTTCACTTAAGACCCAGGGACTTAATCAGCAATATCA ACGTAATAGTTCTGGAACTAAAGGGATCTGAAACAACATTCATGTGTGAATATGC TGATGAGACAGCAACCATTGTAGAATTTCTGAACAGATGGATTACCTTTTGTCAA AGCATCATCTCAACACTGACTTGA (SEQ ID NO: 1). The oligonucleotide encoding native human IL-2 may comprise a sequence that is codon-optimized. The codon-optimized oligonucleotide encoding native human IL-2 may comprise a sequence having at least 90%, 91%, 92%, 93%, 94%, 95%, 96%, 97%, 98%, 99%, or 100% sequence identity to SEQ ID NO: 3. The cell may produce recombinant native human IL-2 protein. The recombinant native human IL-2 protein expressed by the cells may comprise the amino acid sequence of: MYRMQLLSCIALSLALVTNSAPTSSSTKKTQLQLEHLLLDLQMILNGINNYKNPKLT RMLTFKFYMPKKATELKHLQCLEEELKPLEEVLNLAQSKNFHLRPRDLISNINVIVLE LKGSETTFMCEYADETATIVEFLNRWITFCQSIISTLT (SEQ ID NO: 2). The pharmaceutical composition may produce about 1 to about 10, about 1 to about 5, or about 2 to about 4 PCD (picograms/cell/day) of native human IL-2.

The encapsulated cells may comprise ARPE-19 cells comprising the heterologous oligonucleotide molecule. The encapsulated cells may be encapsulated with a polymeric hydrogel. The polymeric hydrogel may comprise chitosan, cellulose, hyaluronic acid, or alginate. The alginate may comprise SLG20. The cells may remain viable for at least 15, 20, 25, or 28 days. The encapsulated cells may not proliferate. The encapsulated cells may produce a sustained amount of IL-2 for at least 5, 10, 15, 20, or 24 hours. The encapsulated cells may produce a sustained amount of IL-2 for up to 30 days.

Thus, in accordance with the present disclosure, there is provided a method of delivering a native cytokine and an additional therapeutic to the subject, the method comprising implanting, or delivering to, the subject a pharmaceutical composition comprising a population of encapsulated cells comprising a heterologous oligonucleotide molecule encoding the native human cytokine and further administering a pharmaceutical composition comprising an additional therapeutic. Also provided is a method of treating a disease or condition, in a subject, the method comprising implanting, or delivering to, the subject a pharmaceutical composition comprising a population of encapsulated cells comprising a heterologous oligonucleotide molecule encoding an IL-2 molecule and further administering a pharmaceutical composition comprising an additional therapeutic. The disease or condition may be a cancer, such as mesothelioma. The mesothelioma may be a pleural mesothelioma, peritoneal mesothelioma, pericardial mesothelioma, testicular mesothelioma, epithelioid mesothelioma, sarcomatoid mesothelioma, biphasic mesothelioma, small cell mesothelioma, deciduoid mesothelioma, cystic and papillary mesothelioma, desmoplastic mesothelioma, adenomatoid mesothelioma, heterologous mesothelioma, well-defined papillary cell mesothelioma, or any combination thereof.

Another embodiment comprises a method of treating mesothelioma in a subject, the method comprising implanting, or delivering to, the subject a pharmaceutical composition comprising a population of encapsulated cells comprising a heterologous oligonucleotide molecule encoding the native human cytokine and further administering a pharmaceutical composition comprising an additional therapeutic. The mesothelioma may be selected from a pleural mesothelioma, peritoneal mesothelioma, pericardial mesothelioma, testicular mesothelioma, epithelioid mesothelioma, sarcomatoid mesothelioma, biphasic mesothelioma, small cell mesothelioma, deciduoid mesothelioma, cystic and papillary mesothelioma, desmoplastic mesothelioma, adenomatoid mesothelioma, heterologous mesothelioma, well-defined papillary cell mesothelioma, or any combination thereof.

The cytokine may be IL-1, IL-1α, IL-1β, IL-1RA, IL-2, IL-4, IL-5, IL-6, IL-7, IL-8, IL-9, IL-10, IL-11, IL-12, IL-12a, IL-12b, IL-13, IL-14, IL-15, IL-16, IL-17, G-CSF, GM-CSF, IL-20, IL-23, IFN-α, IFN-β, IFN-γ, CD154, LT-β, CD70, CD153, CD178, TRAIL, TNF-α, TNF-β, SCF, M-CSF, MSP, 4-1BBL, LIF, OSM, or any combination thereof. The additional therapeutic may be an immunomodulatory agent, such as an inhibitor of PD-1, PD-L1, PD-L2, CTLA4, TIM3, LAG3, VISTA, BTLA, TIGIT, LAIR1, CD73, CD160, 2B4 and/or TGFRβ. The inhibitor may be an anti-PD-1 antibody, anti-PD-L1 antibody, anti-PD-L2 antibody, anti-CTLA4 antibody, anti-TIM3 antibody, anti-LAG3 antibody, anti-VISTA antibody, anti-BTLA antibody, anti-TIGIT antibody, anti-LAIR1 antibody, anti-CD73 antibody, anti-CD160 antibody, anti-2B4 antibody, anti-TGFRβ antibody, or any combination thereof.

In a further embodiment, there is provided is a method of treating mesothelioma in a subject, the method comprising implanting, or delivering to, the subject a pharmaceutical composition comprising a population of encapsulated cells comprising a heterologous oligonucleotide molecule encoding an IL-2 molecule and further administering a pharmaceutical composition comprising an immunomodulatory agent. The mesothelioma may be selected from a pleural mesothelioma, peritoneal mesothelioma, pericardial mesothelioma, testicular mesothelioma, epithelioid mesothelioma, sarcomatoid mesothelioma, biphasic mesothelioma, small cell mesothelioma, deciduoid mesothelioma, cystic and papillary mesothelioma, desmoplastic mesothelioma, adenomatoid mesothelioma, heterologous mesothelioma, well-defined papillary cell mesothelioma, or any combination thereof. The immunomodulatory agent may be an inhibitor of PD-1, PD-L1, PD-L2, CTLA4, TIM3, LAG3, VISTA, BTLA, TIGIT, LAIR1, CD73, CD160, 2B4 and/or TGFRβ, such as an anti-PD-1 antibody, anti-PD-L1 antibody, anti-PD-L2 antibody, anti-CTLA4 antibody, anti-TIM3 antibody, anti-LAG3 antibody, anti-VISTA antibody, anti-BTLA antibody, anti-TIGIT antibody, anti-LAIR1 antibody, anti-CD73 antibody, anti-CD160 antibody, anti-2B4 antibody, anti-TGFRβ antibody, or any combination thereof. The anti-PD-1 antibody may be selected from pembrolizumab, nivolumab, cemiplimab, atezolizumab, dostralimab, durvalumab, avelumab, or any combination thereof.

The treatment may result in activation or increase of immune cells. The activated immune cells may be CD4 and CD8 positive T cells and/or the increased immune cells may be CD4 and CD8 positive effector T cells. The treatment may result in macrophage phenotype shift, such as where the macrophage phenotype shift is from M2-like macrophages to M1-like macrophages. The phenotype shift from M2-like macrophages to M1-like macrophages may result in reduction of M2-like macrophages and increase in M1-like macrophages. The treatment may result in increase in MHC II+ dendritic cells.

In another embodiment, there is provided a method of providing systemic treatment to a subject with cancer, the method comprising implanting in a cavity of the subject a pharmaceutical composition comprising a population of encapsulated cells comprising a heterologous oligonucleotide molecule encoding an IL-2 molecule; and administering an immunomodulatory agent; whereby the pharmaceutical composition stimulates the activation of immune cells in the cavity and the activated immune cells migrate to a region of the subject that is distal to the cavity to treat the cancer systemically in the subject.

In yet another embodiment, there is provided a method of providing systemic treatment to a subject with cancer, the method comprising implanting in a cavity of the subject a pharmaceutical composition comprising a population of encapsulated cells comprising a heterologous oligonucleotide molecule encoding an IL-2 molecule; and administering an immunomodulatory agent; whereby the pharmaceutical composition activates immune cells and the activated immune cells migrate out of the cavity to treat the cancer in the subject.

The cancer is a mesothelioma, such as a pleural mesothelioma, peritoneal mesothelioma, pericardial mesothelioma, testicular mesothelioma, epithelioid mesothelioma, sarcomatoid mesothelioma, biphasic mesothelioma, small cell mesothelioma, deciduoid mesothelioma, cystic and papillary mesothelioma, desmoplastic mesothelioma, adenomatoid mesothelioma, heterologous mesothelioma, well-defined papillary cell mesothelioma, or any combination thereof. The immunomodulatory agent may be an inhibitor of PD-1, PD-L1, PD-L2, CTLA4, TIM3, LAG3, VISTA, BTLA, TIGIT, LAIR1, CD73, CD160, 2B4 and/or TGFRβ. The inhibitor may be an anti-PD-1 antibody, anti-PD-L1 antibody, anti-PD-L2 antibody, anti-CTLA4 antibody, anti-TIM3 antibody, anti-LAG3 antibody, anti-VISTA antibody, anti-BTLA antibody, anti-TIGIT antibody, anti-LAIR1 antibody, anti-CD73 antibody, anti-CD160 antibody, anti-2B4 antibody, anti-TGFRβ antibody, or any combination thereof. The anti-PD-1 antibody may be selected from pembrolizumab, nivolumab, cemiplimab, atezolizumab, dostralimab, durvalumab, avelumab, or any combination thereof.

The activated immune cells may be CD4 and CD8 positive T cells. The cavity may be a pleural cavity and/or pleural cavity.

For any of the foregoing methods, the additional therapeutic or the immunomodulatory agent may be administered 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days following implantation of the pharmaceutical composition comprising a population of encapsulated cells. The additional therapeutic or the immunomodulatory agent may be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days following implantation of the pharmaceutical composition comprising a population of encapsulated cells. The subject may be administered about 0.01 μg/kg/day to about 20 μg/kg/day, about 0.1 μg/kg/day to about 20 μg/kg/day, about 1 μg/kg/day to about 20 μg/kg/day, about 2 μg/kg/day to about 20 μg/kg/day, about 5 μg/kg/day to about 20 μg/kg/day, about 7.5 to about 20 μg/kg/day, about 9 μg/kg/day to about 20 μg/kg/day, about 10 μg/kg/day to about 20 μg/kg/day, about 11 μg/kg/day to about 20 μg/kg/day, about 12 μg/kg/day to about 20 μg/kg/day, about 13 μg/kg/day to about 20 μg/kg/day, about 14 μg/kg/day to about 15 μg/kg/day, about 15 μg/kg/day to about 20 μg/kg/day, about 10 μg/kg/day to about 15 μg/kg/day, about 11 μg/kg/day to about 15 μg/kg/day, about 12 μg/kg/day to about 15 μg/kg/day, about 13 μg/kg/day to about 15 μg/kg/day, about 14 μg/kg/day to about 15 μg/kg/day, about 16 μg/kg/day to about 20 μg/kg/day, about 17 μg/kg/day to about 20 μg/kg/day, about 18 μg/kg/day to about 20 μg/kg/day, about 0.01 μg/kg/day, about 0.1 μg/kg/day, about 1 μg/kg/day, about 2 μg/kg/day, about 3 μg/kg/day, about 4 μg/kg/day, about 5 μg/kg/day, about 6 μg/kg/day, about 7 μg/kg/day, about 8 μg/kg/day, about 9 μg/kg/day, about 10 μg/kg/day, about 11 μg/kg/day, about 12 μg/kg/day, about 13 μg/kg/day, about 14 μg/kg/day, about 15 μg/kg/day, about 6 μg/kg/day, about 17 μg/kg/day, about 18 μg/kg/day, about 19 μg/kg/day, or about 20 μg/kg/day, of the encapsulated cells. The pharmaceutical composition may be implanted according to a method or using a device as provided for herein. The IL-2 molecule may be a native human IL-2 or an IL-2 mutein. The heterologous oligonucleotide encoding the native human IL-2 may comprise a sequence of:

(SEQ ID NO: 1)
ATGTACAGGATGCAACTCCTGTCTTGCATTGCACTAAGTCTTGCACTTGT
CACAAACAGTGCACCTACTTCAAGTTCTACAAAGAAAACACAGCTACAAC
TGGAGCATTTACTGCTGGATTTACAGATGATTTTGAATGGAATTAATAAT
TACAAGAATCCCAAACTCACCAGGATGCTCACATTTAAGTTTTACATGCC
CAAGAAGGCCACAGAACTGAAACATCTTCAGTGTCTAGAAGAAGAACTCA
AACCTCTGGAGGAAGTGCTAAATTTAGCTCAAAGCAAAAACTTTCACTTA
AGACCCAGGGACTTAATCAGCAATATCAACGTAATAGTTCTGGAACTAAA
GGGATCTGAAACAACATTCATGTGTGAATATGCTGATGAGACAGCAACCA
TTGTAGAATTTCTGAACAGATGGATTACCTTTTGTCAAAGCATCATCTCA
ACACTGACTTGA

The heterologous oligonucleotide encoding the native human IL-2 comprises a sequence that is codon-optimized. The population of encapsulated cells of claim 39, wherein the codon-optimized oligonucleotide encoding native human IL-2 comprises a sequence having at least 90%, 91%, 92%, 93%, 94%, 95%, 96%, 97%, 98%, 99%, or 100% sequence identity to SEQ ID NO: 3.

For any of the foregoing methods, the cell may produce recombinant native human IL-2 protein. The recombinant native human IL-2 protein expressed by the cells may comprise the amino acid sequence of:

(SEQ ID NO: 2)
MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT

The IL-2 mutein may comprise an amino acid sequence having at least 75%, 80%, 85%, 90%, 91%, 92%, 93%, 94%, 95%, 96%, 97%, 98%, 99%, or 100% identity to the amino acid sequence of:

(SEQ ID NO: 2)
MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT

The pharmaceutical composition may produce about 1 to about 10, about 1 to about 5, or about 2 to about 4 PCD (picograms/cell/day) of native human IL-2. The encapsulated cells may comprise a cell as provided for herein. The encapsulated cells may comprise ARPE-19 cells comprising the heterologous oligonucleotide molecule. The encapsulated cells may be encapsulated with a polymeric hydrogel, such as a polymeric hydrogel comprising chitosan, cellulose, hyaluronic acid, or alginate. The polymeric hydrogel may comprise alginate, such as SLG20, for example, SLG20 at about 0.1%-3% SLG20. The cells may remain viable for at least 15, 20, 25, or 28 days. The encapsulated cells may not proliferate. The encapsulated cells may produce a sustained amount of IL-2 for at least 5, 10, 15, 20, or 24 hours. The encapsulated cells may produce a sustained amount of IL-2 for up to 30 days.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

It is contemplated that any method or composition described herein can be implemented with respect to any other method or composition described herein.

Other objects, features and advantages of the present disclosure will become apparent from the following detailed description. It should be understood, however, that the detailed description and the specific examples, while indicating certain embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-K. Dose response of RPE-mIL2 in a peritoneal model of MM. FIG. 1A, Schematic demonstrating the development of RPE-mIL2 cells and encapsulation of them in hydrogel spheres. FIG. 1B, Enzyme-linked immuno-sorbent assay (ELISA) measurements of mIL2 in supernatant from capsules after 24 hours of in vitro culture. FIG. 1C, Representative live/dead image of RPE-mIL2 cells encapsulated at 42e6 cells/mL alginate. FIG. 1D, Schematic illustrating the experimental timeline for tumor establishment, treatment, and IVIS imaging. FIG. 1E, Luminescent images tracking AB1-FLuc tumor burden over time beginning at day 6 post injection, and weekly until day 28 post injection. FIGS. 1F-K, Quantification of tumor burden for each treatment group (n=5-7) represented by total flux (photons/s) plotted over time. Black arrows indicate the day of treatment administration.

FIGS. 2A-E. Dose dependent anti-tumor effects of RPE-mIL2 administration. FIG. 2A, Darkfield images of RPE-mIL2 cells encapsulated at 10.5, 21, and 42e6 cells/mL of SLG20 alginate. Images acquired at 2× magnification; scale bar is 2000 m. FIGS. 2B-E, Plots tracking the weight of individual mice in each of the RPE-mIL2 treatment groups over the course of treatment.

FIGS. 3A-B. CD8+ T cells are needed for a robust anti-tumor response in mice with MPM tumors. FIG. 3A, Luminescent images tracking AB1-Fluc tumor burden acquired 6 days and 13 days post tumor injection. Mice were injected intraperitoneally with anti-CD8 and anti-CD4, or isotype control antibodies 2 days prior to, on, and 2 days following tumor injection.

FIG. 3B, Quantification of luminescent images represented by total flux (photons per second) for each treatment group (n=5-6).

FIGS. 4A-J. RPE-mIL2 improves therapeutic efficacy of aPD1. FIG. 4A, Schematic of the experimental timeline for BALB/c tumor establishment, treatment administration, and IVIS imaging. FIG. 4B, Luminescent images tracking tumor burden over time. FIGS. 4C-G, Quantification of tumor burden for each treatment group (n=7-8) represented by total flux (photons/s) plotted over time. Black arrows indicate the day of treatment administration. FIG. 411, Survival curves plotted as percent survival over time beginning after tumor injection (n=7-8). P value was determined by a comparison of survival curves by the log-rank (Mantel-Cox) test, ns=not significant. FIG. 4I, plot of subcutaneous tumor volume over time in Naive mice compared to RPE-mIL2+PD1 treated mice. P values were acquired using one-way ANOVA with Holm-Sidak method for multiple comparisons. FIG. 4J, Representative macroscopic images of the left flank 28 days post subcutaneous tumor injection. (Left; Naive, Right; RPE-mIL2PD1 treated).

FIGS. 5A-E. Combination treatment did not cause significant deviations in body weight. Plots tracking the weight of individual mice in each of the treatment groups over the course of treatment.

FIGS. 6A-B. Alteration of immune composition after RPE-mIL2 or RPE-mIL2+aPD1 therapy. FIG. 6A, Immune atlas map. CyTOF was performed with single cell suspension obtained from peritoneal lavage fluid. The Uniform Manifold Approximation and Projection (UMAP) was applied for dimensional reduction with 1,000,000 cells (40,000/each experiment×4 mice/group×4 groups). Fourteen phenotypes were analyzed. IL-2 treatment and combination of anti-PD-1 therapy with IL-2 led to dramatic changes in lymphocytes and myeloid cell compositions. FIG. 6B, Comparison of T cells, macrophages, and B cells across treatment groups (n=4 per group). P values were acquired using one way ANOVA with Holm-Sidak method for multiple comparisons, ns=not significant.

FIGS. 7A-E. Alteration of immune cell subsets after RPE-mIL2 or RPE-mIL2+aPD1 therapy. FIG. 7A, Comparison of M1-like and M2-like macrophages across treatment groups. Expression of CD40 among M1-like or M2-like macrophages across treatment groups. FIG. 7B, Comparison of cDC cells across treatment groups. Expression of CD40 among cDC cells across treatment groups. FIG. 7C, Comparison of naïve and memory B cells across treatment groups. FIG. 7D, Comparison of naïve, activated, or effector memory CD4+ T cells across treatment groups. Expression of IFNg among activated CD4+ T cells across treatment groups. FIG. 7E, Comparison of naïve, activated, or effector memory CD8+ T cells across treatment groups. Expression of PD-1 among activated C84+ T cells across treatment groups (n=4 per group). P values were acquired using one-way ANOVA with Holm-Sidak method for multiple comparisons, ns=not significant.

FIGS. 8A-C. RPE-hIL2 Pharmacokinetics in immunocompetent mice. FIG. 8A, In vivo hIL2 concentrations (ng/mL) in IP fluid and blood as a function of time with a fixed dose of 50 capsules (n=5 mice per group). Values plotted are mean +/− SEM. FIG. 8B, Capsules collected from pharmacokinetic studies were imaged using brightfield microscopy (2×) to qualitatively monitor PFO of the alginate core-shell surface over time. Single field of view (2×) images with overlapping fields were stitched to create a mosaic. FIG. 8C, H&E staining of explanted capsules at various timepoints. Capsule sections shown at 5× and 20× magnification. Host immune cells accumulated on the surface of the capsules over time (black arrows).

FIGS. 9A-G. Evaluation of human IL-2 kinetics in the rat pleura. FIG. 9A, Macroscopic image of the pleural cavity 24 hours post-implant. White arrows indicate RPE-hIL2. FIG. 9B, Enzyme-linked immuno-sorbent assay (ELISA) measurements of hIL2 concentration in the pleural cavity and blood at 24 hours, 7 days, 21 days, and 30 days post implant. FIG. 9C, Bright field images of capsules retrieved from the pleural cavity 30 days post implant. Image acquired at 2× magnification. FIGS. 9D-G, plots of white blood cells, red blood cell, monocyte, and platelets derived from complete blood count analysis at 24 hours, 7 days, and 30 days post implant (n=5 per group). Values were compared to untreated controls. Differences in cell count were not significant across all groups. P values were acquired using one-way ANOVA with Holm-Sidak method for multiple comparisons.

FIGS. 10A-I. Toxicological analysis of hIL2 in the rat pleura over the span of 30 days. FIG. 10A, Representative images of H&E-stained sections of the kidney, liver, spleen, and lungs of untreated control rats compared to rats sacrificed at 30 days post capsule implant (n=5). FIG. 10B, Plot tracking the weight of individual rats over the course of treatment. FIGS. 10C-D, evaluation of general health through changes in insulin and glucose for each time point. Values are compared to untreated controls. Differences in values were not significant across all groups. P values were acquired using one-way ANOVA with Holm-Sidak method for multiple comparisons. FIG. 10E, evaluation of general health through changes in Triglycerides for each time point. FIGS. 10F-G, evaluation of heart health through changes in HDL and LDL. FIGS. 10H-I, evaluation of liver health through changes in ATL and AST. All values were compared to untreated controls. Differences in values were not significant across all groups. P values were acquired using one-way ANOVA with Holm-Sidak method for multiple comparisons.

### DETAILED DESCRIPTION

The present disclosure features implantable constructs for delivery of native human IL-2 to a subject in a controlled release manner, and related methods of use thereof. These embodiments will be described below in more detail.

### I. Definitions

“Cell,” as used herein, refers to an individual cell. In an embodiment, a cell is a primary cell or is derived from a cell culture. In an embodiment, a cell is a stem cell or is derived from a stem cell. A cell may be xenogeneic, autologous, or allogeneic. In an embodiment, a cell is engineered (e.g., genetically engineered) or is not engineered (e.g., not genetically engineered). In some embodiments, the cell is an APRE-19 cell. In some embodiments, the cell expresses native human IL-2 protein.

“Prevention,” “prevent,” and “preventing” as used herein refers to a treatment that comprises administering or applying a therapy, e.g., administering an implantable construct (e.g., as described herein) comprising a therapeutic agent (e.g., a therapeutic agent described herein) prior to the onset of a disease or condition in order to preclude the physical manifestation of said disease or condition. In some embodiments, “prevention,” “prevent,” and “preventing” require that signs or symptoms of the disease or condition have not yet developed or have not yet been observed. In some embodiments, treatment comprises prevention and in other embodiments it does not. In some embodiments, the prevention is the prevention of the recurrence of a disease, such as a tumor (cancer) after the tumor or cancer has been eradicated by an initial treatment.

“Subject,” as used herein, refers to the recipient of the implantable construct described herein. The subject may include a human and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development (e.g., a male or female of any age group, e.g., a pediatric subject (e.g., infant, child, adolescent) or adult subject (e.g., young adult, middle-aged adult, or senior adult). A non-human animal may be a transgenic animal.

“Treatment,” “treat,” and “treating,” as used herein, refer to reversing, alleviating, delaying the onset of, or inhibiting the progress of one or more of a symptom, manifestation, or underlying cause of a disease or condition. (e.g., as described herein), e.g., by administering or applying a therapy, e.g., administering an implantable construct comprising a therapeutic agent (e.g., a therapeutic agent described herein). In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease or condition. In an embodiment, treating comprises reducing, reversing, alleviating, reducing, or delaying the onset of, an underlying cause of a disease or condition. In some embodiments, “treatment,” “treat,” and “treating” require that signs or symptoms of the disease or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

### B. CELLS

Implantable constructs described herein may contain a cell, for example, an engineered cell. A cell be derived from any mammalian organ or tissue, including the brain, nerves, ganglia, spine, eye, heart, liver, kidney, lung, spleen, bone, thymus, lymphatic system, skin, muscle, pancreas, stomach, intestine, blood, ovary, uterus, or testes. In some embodiments, the cell is a APRE-19 cell.

A cell may be derived from a donor (e.g., an allogeneic cell), derived from a subject (e.g., an autologous cell), or from another species (e.g., a xenogeneic cell). In an embodiment, a cell can be grown in cell culture, or prepared from an established cell culture line, or derived from a donor (e.g., a living donor or a cadaver). In an embodiment, a cell is genetically engineered. In another embodiment, a cell is not genetically engineered. A cell may include a stem cell, such as a reprogrammed stem cell, or an induced pluripotent cell. Exemplary cells include mesenchymal stem cells (MSCs), fibroblasts (e.g., primary fibroblasts). HEK cells (e.g., HEK293T), Jurkat cells, HeLa cells, retinal pigment epithelial (RPE) cells, HUVEC cells, NIH3T3 cells, CHO-K1 cells, COS-1 cells, COS-7 cells, PC-3 cells, HCT 116 cells, A549MCF-7 cells, HuH-7 cells, U-2 OS cells, HepG2 cells, Neuro-2a cells, and SF9 cells. In an embodiment, a cell for use in an implantable construct is an RPE cell.

A cell included in an implantable construct may produce or secrete a therapeutic agent, such as native human IL-2. In an embodiment, a cell included in an implantable construct may produce or secrete a single type of therapeutic agent or a plurality of therapeutic agents. In an embodiment, an implantable construct may comprise a cell that is transduced or transfected with a nucleic acid (e.g., a vector) comprising an expression sequence of a therapeutic agent. For example, a cell may be transduced or transfected with a lentivirus. A nucleic acid introduced into a cell (e.g., by transduction or transfection) may be incorporated into a nucleic acid delivery system, such as a plasmid, or may be delivered directly. In an embodiment, a nucleic acid introduced into a cell (e.g., as part of a plasmid) may include a region to enhance expression of the therapeutic agent and/or to direct targeting or secretion, for example, a promoter sequence, an activator sequence, or a cell-signaling peptide, or a cell export peptide. Exemplary promoters include EF-1a, CMV, Ubc, hPGK, VMD2, and CAG. Exemplary activators include the TET1 catalytic domain, P300 core, VPR, rTETR, Cas9 (e.g., from S. pyogenes or S. aureus), and Cpf1 (e.g., from L. bacterium).

An implantable construct described herein may comprise a cell or a plurality of cells. In the case of a plurality of cells, the concentration and total cell number may be varied depending on a number of factors, such as cell type, implantation location, and expected lifetime of the implantable construct. In an embodiment, the total number of cells included in an implantable construct is greater than about 2, 4, 6, 8, 10, 20, 30, 40, 50, 75, 100, 200, 250, 500, 750, 1000, 1500, 2000, 5000, 10000, or more. In an embodiment, the total number of cells included in an implantable construct is greater than about 1.0×10², 1.0×10³, 1.0×10⁴, 1.0×10⁵, 1.0×10⁶, 1.0×10⁷, 1.0×10⁸, 1.0×10⁹, 1.0×10¹⁰, or more. In an embodiment, the total number of cells included in an implantable construct is less than about than about 10000, 5000, 2500, 2000, 1500, 1000, 750, 500, 250, 200, 100, 75, 50, 40, 30, 20, 10, 8, 6, 4, 2, or less. In an embodiment, the total number of cells included in an implantable construct is less than about 1.0×10¹⁰, 1.0×10⁹, 1.0×10⁸, 1.0×10⁷, 1.0×10⁶, 1.0×10⁵, 1.0×10⁴, 1.0×10³, 1.0×10², or less. In an embodiment, a plurality of cells is present as an aggregate. In an embodiment, a plurality of cells is present as a cell dispersion.

Specific features of a cell contained within an implantable construct may be determined, e.g., prior to and/or after incorporation into the implantable construct. For example, cell viability, cell density, or cell expression level may be assessed. In an embodiment, cell viability, cell density, and cell expression level may be determined using standard techniques, such as cell microscopy, fluorescence microscopy, histology, or biochemical assay.

### C. THERAPEUTIC AGENTS

An implantable construct described herein may contain a therapeutic agent, for example, produced or secreted by a cell, such as native human IL-2. A therapeutic agent may include a nucleic acid encoding the protein (e.g., an RNA, a DNA, or an oligonucleotide), a protein (e.g., an antibody, enzyme, cytokine, hormone, receptor) that is secreted from the cell, and the like. In an embodiment, the implantable construct comprises a cell or a plurality of cells that are genetically engineered to produce or secrete a therapeutic agent.

In some embodiments, native human IL-2 refers to a protein encoded by a nucleic acid sequence comprising

(SEQ ID NO: 1)
ATGTACAGGATGCAACTCCTGTCTTGCATTGCACTAAGTCTTGCACTTGT
CACAAACAGTGCACCTACTTCAAGTTCTACAAAGAAAACACAGCTACAAC
TGGAGCATTTACTGCTGGATTTACAGATGATTTTGAATGGAATTAATAAT
TACAAGAATCCCAAACTCACCAGGATGCTCACATTTAAGTTTTACATGCC
CAAGAAGGCCACAGAACTGAAACATCTTCAGTGTCTAGAAGAAGAACTCA
AACCTCTGGAGGAAGTGCTAAATTTAGCTCAAAGCAAAAACTTTCACTTA
AGACCCAGGGACTTAATCAGCAATATCAACGTAATAGTTCTGGAACTAAA
GGGATCTGAAACAACATTCATGTGTGAATATGCTGATGAGACAGCAACCA
TTGTAGAATTTCTGAACAGATGGATTACCTTTTGTCAAAGCATCATCTCA
ACACTGACTTGA.

In some embodiments, the nucleic acid coding sequence encoding native human IL-2 is codon-optimized. In some embodiments, the nucleic acid coding sequence encoding native human IL-2 is codon-optimized for expression in a mammalian cell. The codon optimized sequence may be generated using a commercially available algorithm, e.g., GeneOptimizer (ThermoFisher Scientific), OptimumGene™ (GenScript, Piscataway, NJ USA), GeneGPS® (ATUM, Newark, CA USA), or Java Codon Adaptation Tool (JCat, http://www.jcat.de, Grote, A. et al., Nucleic Acids Research, Vol 33, Issue suppl_2, pp. W526-W531 (2005), IDT Codon Optimization Tool (Integrated DNA Technologies), VectorBuilder Codon Optimization tool (VectorBuilder Inc.), Codon Optimization OnLine (COOL, http://bioinfo.bti.a-star.edu.sg/COOL/; Chin J. X., et al., Bioinformatics, Vol 30, Issue 15, p. 2210-2212 (2014)), or ExpOptimizer (NovoPro, Shanghai, China). Examples of codon-optimized nucleic acid coding sequences encoding native human IL-2 comprise, but are not limited to:

(SEQ ID NO: 3)
ATGTACCGGATGCAGCTGCTGTCCTGCATCGCACTGTCCCTCGCCCTGGT
GACAAATTCTGCCCCCACCTCCTCCAGCACAAAAAAGACCCAGTTGCAGC
TGGAGCACCTGCTGCTGGATCTGCAGATGATCCTGAATGGCATCAATAAC
TACAAAAACCCTAAACTGACCAGAATGCTGACCTTTAAATTTTACATGCC
TAAAAAGGCAACCGAGCTGAAGCACCTGCAGTGCCTGGAAGAGGAACTGA
AGCCCCTGGAGGAGGTGCTGAACCTGGCCCAGAGCAAGAACTTTCACCTG
CGGCCCCGCGACCTGATCAGCAACATCAACGTGATCGTGCTGGAGCTGAA
GGGCAGTGAAACCACATTCATGTGCGAGTACGCCGACGAGACCGCCACAA
TCGTGGAGTTCCTGAACAGATGGATCACATTCTGTCAGTCCATCATTAGC
ACACTGACCTAA;
(SEQ ID NO: 4)
ATGTACCGCATGCAGCTGCTGAGCTGCATCGCCCTGAGCCTGGCCCTGGT
GACCAACAGCGCCCCCACCAGCAGCAGCACCAAGAAGACCCAGCTGCAGC
TGGAGCACCTGCTGCTGGACCTGCAGATGATCCTGAACGGCATCAACAAC
TACAAGAACCCCAAGCTGACCCGCATGCTGACCTTCAAGTTCTACATGCC
CAAGAAGGCCACCGAGCTGAAGCACCTGCAGTGCCTGGAGGAGGAGCTGA
AGCCCCTGGAGGAGGTGCTGAACCTGGCCCAGAGCAAGAACTTCCACCTG
CGCCCCCGCGACCTGATCAGCAACATCAACGTGATCGTGCTGGAGCTGAA
GGGCAGCGAGACCACCTTCATGTGCGAGTACGCCGACGAGACCGCCACCA
TCGTGGAGTTCCTGAACCGCTGGATCACCTTCTGCCAGAGCATCATCAGC
ACCCTGACCTAA;
(SEQ ID NO: 5)
ATGTATAGGATGCAGCTGCTCTCTTGTATCGCGTTGTCTCTGGCTTTGGT
GACTAACTCAGCTCCCACGTCCAGCAGTACCAAAAAGACCCAGCTGCAGC
TGGAACATCTTCTGTTGGATCTGCAAATGATACTGAATGGGATCAACAAC
TATAAAAACCCAAAACTGACTAGAATGCTGACTTTCAAGTTCTACATGCC
TAAAAAGGCAACAGAATTGAAGCACCTTCAGTGCCTGGAGGAGGAGCTTA
AGCCCCTGGAGGAGGTGCTGAATCTGGCCCAAAGTAAGAATTTTCATCTG
CGACCCAGGGATCTGATCAGTAATATCAATGTGATCGTCCTGGAGCTGAA
GGGCAGTGAGACCACGTTTATGTGTGAATACGCAGACGAAACCGCCACTA
TCGTTGAATTCTTGAACAGGTGGATCACCTTTTGTCAGAGTATCATCAGC
ACCCTCACT;
or
(SEQ ID NO: 6)
ATGTACAGAATGCAGCTGCTGAGCTGCATCGCCCTGAGCCTGGCCCTGGT
GACCAACAGCGCCCCCACAAGCAGCAGCACCAAGAAGACACAGCTGCAGC
TGGAGCACCTGCTGCTGGACCTGCAGATGATCCTGAACGGCATCAACAAC
TACAAGAACCCCAAGCTGACAAGAATGCTGACCTTCAAGTTCTACATGCC
CAAGAAGGCCACCGAGCTGAAGCACCTGCAGTGCCTGGAGGAGGAGCTGA
AGCCCCTGGAAGAGGTGCTGAACCTGGCTCAGAGCAAGAACTTCCACCTG
AGACCTAGAGACCTGATCAGCAACATCAACGTGATCGTGCTGGAGCTGAA
GGGCAGCGAGACCACCTTCATGTGCGAGTACGCCGACGAGACCGCCACCA
TCGTGGAGTTCCTGAACAGATGGATCACCTTCTGTCAGAGCATCATCAGC
ACCCTGACCTGA.

In some embodiments, the codon-optimized nucleic acid coding sequence encoding native human IL-2 comprise the nucleic acid sequence as set forth in SEQ ID NO: 3-6. In some embodiments, the codon-optimized nucleic acid coding sequence encoding native human IL-2 comprise the nucleic acid sequence as set forth in SEQ ID NO: 3. In some embodiments, the codon-optimized nucleic acid coding sequence encoding native human TL-2 comprise the nucleic acid sequence having at least 90%, 92%, 93%, 94%, 95%, 96%, 97%, 98%, 99%, or 100% identity to the sequence of SEQ ID NO: 3-6. In some embodiments, the codon-optimized nucleic acid coding sequence encoding native human IL-2 comprise the nucleic acid sequence having at least 90%, 92%, 93%, 94%, 95%, 96%, 97%, 98%, 99%, or 100% identity to the sequence of SEQ ID NO: 3.

In some embodiments, the native human protein produced by the cell is formed from the formed from an amino acid sequence of:

(SEQ ID NO: 2)
MYRMQLLSCIALSLALVINSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTEMCEYADETATIVEFLNRWITFCQSIIS
TLT.

Without being bound by any particular theory, the native IL-2 produced by the cells comprising the nucleic acid sequence of SEQ ID NO: 1 differs from recombinant IL-2 produced from bacteria or other non-eukaryotic cells due to differences in post-translational modification. Thus, the cells expressing the native human IL-2 is superior because it has superior potency. This is described herein in the Examples section. In some embodiments, the IL-2 is a IL-2 mutein or a modified IL-2 molecule, fusion proteins or antibodies that act on the IL-2 pathway. In some embodiments, the IL-2 is a pegylated IL-2 molecule. In some embodiments, the pegylated IL-2 molecule has a wild-type sequence. In some embodiments, the pegylated IL-2 has a mutant IL-2 sequence. In some embodiments, the capsules or cells are used to produce NKTR-214 (pegylated IL-2; Clin Cancer Res Feb. 1, 2016 (22) (3) 680-690; DOI: 10.1158/1078-0432.CCR-15-1631, which is hereby incorporated by reference in its entirety), THOR-707 (SAR444245; Annals of Oncology, Volume 30, Supplement 5, October 2019, Page v501, which is hereby incorporated by reference in its entirety), ALKS 4230 (J Immunother Cancer. 2020 April; 8(1):e000673. doi: 10.1136/jitc-2020-000673, Nemvaleukin Alfa), TransCon IL-2 β/γ, BNT151, BNT153, CLN-617, CUE-101, CUE-102, CUE-103, Anktiva® (N-803), KY1043, MDNA11, NL-201, SO-C101, R06874281, Simlukafusp Alfa, RG7461, WTX-124, WTX-330, XTX202, or XTX401 or any combination thereof.

In some embodiments, the IL-2 mutein comprises an amino acid sequence having at least 75%, 80%, 85%, 90%, 91%, 92%, 93%, 94%, 95%, 96%, 97%, 98%, 99%, or 100% identity to the amino acid sequence of:

(SEQ ID NO: 2)
MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT.

The cell can also be modified to produce or secrete an additional protein or molecule in addition to native human IL-2. In some embodiments, the additional protein or molecule is a IL-2 mutein or a modified IL-2 molecule, fusion proteins or antibodies that act on the IL-2 pathway. In some embodiments, the IL-2 is a pegylated IL-2 molecule. In some embodiments, the pegylated IL-2 molecule has a wild-type sequence. In some embodiments, the pegylated IL-2 has a mutant IL-2 sequence. In some embodiments, the additional protein or molecule is selected from NKTR-214, THOR-707, ALKS 4230, Nemvaleukin Alfa, TransCon IL-2 β/γ, BNT151, BNT153, CLN-617, CUE-101, CUE-102, CUE-103, Anktiva® (N-803), KY1043, MDNA11, NL-201, SO-C101, R06874281, Simlukafusp Alfa, RG7461, WTX-124, WTX-330, XTX202, XTX401. The additional protein may be of any size, e.g., greater than about 100 Da, 200 Da, 250 Da, 500 Da, 750 Da, 1 KDa, 1.5 kDa, 2 kDa, 2.5 kDa, 3 kDa, 4 kDa, 5 kDa, 6 kDa, 7 kDa, 8 kDa, 9 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 95 kDa, 100 kDa, 125 kDa, 150 kDa, 200 kDa, 200 kDa, 250 kDa, 300 kDa, 400 kDa, 500 kDa, 600 kDa, 700 kDa, 800 Da, 900 kDa, or more. In an embodiment, the protein is composed of a single subunit or multiple subunits (e.g., a dimer, trimer, tetramer, etc.). A protein produced or secreted by a cell may be modified, for example, by glycosylation, methylation, or other known natural or synthetic protein modification. A protein may be produced or secreted as a pre-protein or in an inactive form and may require further modification to convert it into an active form.

Proteins produced or secreted by a cell may be include antibodies or antibody fragments, for example, an Fc region or variable region of an antibody. Exemplary antibodies include anti-PD-1, anti-PD-L1, anti-CTLA4, anti-TNFα, and anti-VEGF antibodies. An antibody may be monoclonal or polyclonal. Other exemplary proteins include a lipoprotein, an adhesion protein, hemoglobin, enzymes, proenkephalin, a growth factor (e.g., EGF, IGF-1, VEGF alpha, HGF, TGF beta, bFGF), or a cytokine.

A protein produced or secreted by a cell may also include a hormone. Exemplary hormones include growth hormone, growth hormone releasing hormone, prolactin, lutenizing hormone (LH), anti-diuretic hormone (ADH), oxytocin, thyroid stimulating hormone (TSH), thyrotropin-release hormone (TRH), adrenocorticotropic hormone (ACTH), follicle-stimulating hormone (FSH), thyroxine, calcitonin, parathyroid hormone, aldosterone, cortisol, epinephrine, glucagon, insulin, estrogen, progesterone, and testosterone.

A protein produced or secreted by a cell may include other cytokines. A cytokine may be a pro-inflammatory cytokine or an anti-inflammatory cytokine. Example of cytokines include IL-1, IL-1α, IL-1β, IL-1RA, IL-4, IL-5, IL-6, IL-7, IL-8, IL-9, IL-10, IL-11, IL-12, IL-12a, IL-12b, IL-13, IL-14, IL-15, IL-16, IL-17, G-CSF, GM-CSF, IL-20, IL-23, IFN-α, IFN-β, IFN-γ, CD154, LT-β, CD70, CD153, CD178, TRAIL, TNF-α, TNF-β, SCF, M-CSF, MSP, 4-1BBL, LIF, OSM, and others. For example, a cytokine may include any cytokine described in M. J. Cameron and D. J. Kelvin, Cytokines, Chemokines, and Their Receptors (2013), Landes Biosciences, which is incorporated herein by reference in its entirety.

An provided for herein implantable construct may comprise a cell expressing a single type of therapeutic agent, e.g., a single protein or nucleic acid, or may express more than one type of therapeutic agent, e.g., a plurality of proteins or nucleic acids. In an embodiment, an implantable construct comprises a cell expressing two types of therapeutic agents (e.g., two types of proteins or nucleic acids). In an embodiment, an implantable construct comprises a cell expressing three types of therapeutic agents (e.g., three types of proteins or nucleic acids). In an embodiment, an implantable construct comprises a cell expressing four types of therapeutic agents (e.g., four types of proteins or nucleic acids).

In an embodiment, an implantable construct comprises a cell expressing a single type of nucleic acid (e.g., DNA or RNA) or may express more than one type of nucleic acid, e.g., a plurality of nucleic acid (e.g., DNA or RNA). In an embodiment, an implantable construct comprises a cell expressing two types of nucleic acids (e.g., DNA or RNA). In an embodiment, an implantable construct comprises a cell expressing three types of nucleic acids (e.g., DNA or RNA). In an embodiment, an implantable construct comprises a cell expressing four types of nucleic acids (e.g., DNA or RNA).

In an embodiment, an implantable construct comprises a cell expressing a single type of protein, or may express more than one type of protein, e.g., a plurality of proteins. In an embodiment, an implantable construct comprises a cell expressing two types of proteins. In an embodiment, an implantable construct comprises a cell expressing three types of proteins. In an embodiment, an implantable construct comprises a cell expressing four types of proteins.

In an embodiment, an implantable construct comprises a cell expressing a single type of enzyme, or may express more than one type of enzyme, e.g., a plurality of enzymes. In an embodiment, an implantable construct comprises a cell expressing two types of enzymes. In an embodiment, an implantable construct comprises a cell expressing three types of enzymes. In an embodiment, an implantable construct comprises a cell expressing four types of enzymes.

In an embodiment, an implantable construct comprises a cell expressing a single type of antibody or antibody fragment or may express more than one type of antibody or antibody fragment, e.g., a plurality of antibodies or antibody fragments. In an embodiment, an implantable construct comprises a cell expressing two types of antibodies or antibody fragments. In an embodiment, an implantable construct comprises a cell expressing three types of antibodies or antibody fragments. In an embodiment, an implantable construct comprises a cell expressing four types of antibodies or antibody fragments.

In an embodiment, an implantable construct comprises a cell expressing a single type of hormone, or may express more than one type of hormone, e.g., a plurality of hormones. In an embodiment, an implantable construct comprises a cell expressing two types of hormones. In an embodiment, an implantable construct comprises a cell expressing three types of hormones. In an embodiment, an implantable construct comprises a cell expressing four types of hormones.

In an embodiment, an implantable construct comprises a cell expressing a single type of enzyme, or may express more than one type of enzyme, e.g., a plurality of enzymes. In an embodiment, an implantable construct comprises a cell expressing two types of enzymes. In an embodiment, an implantable construct comprises a cell expressing three types of enzymes. In an embodiment, an implantable construct comprises a cell expressing four types of enzymes.

In an embodiment, an implantable construct comprises a cell expressing a single type of cytokine or may express more than one type of cytokine, e.g., a plurality of cytokines. In an embodiment, an implantable construct comprises a cell expressing two types of cytokines. In an embodiment, an implantable construct comprises a cell expressing three types of cytokines. In an embodiment, an implantable construct comprises a cell expressing four types of cytokines.

In some embodiments, an additional therapeutic is administered in addition to the implantable construct described herein. In some embodiments, a therapeutic agent, for example, produced or secreted by a cell, such as native human IL-2 is delivered to the subject and the subject is further administered an additional therapeutic. In some embodiments, the additional therapeutic is an immunomodulatory agent. In some embodiments, the immunomodulatory agent is an inhibitor of PD-1, PD-L1, PD-L2, CTLA4, TIM3, LAG3, VISTA, BTLA, TIGIT, LAIR1, CD73, CD160, 2B4 and/or TGFRβ. In some embodiments, the inhibitor is an anti-PD-1 antibody, anti-PD-L1 antibody, anti-PD-L2 antibody, anti-CTLA4 antibody, anti-TIM3 antibody, anti-LAG3 antibody, anti-VISTA antibody, anti-BTLA antibody, anti-TIGIT antibody, anti-LAIR1 antibody, anti-CD73 antibody, anti-CD160 antibody, anti-2B4 antibody, anti-TGFRβ antibody, or any combination thereof. In some embodiments, the inhibitor of PD-1 is an anti-PD-1 antibody. In some embodiments, the inhibitor of PD-L1 is an anti-PD-L1 antibody. In some embodiments, the inhibitor of PD-L2 is an anti-PD-L2 antibody. In some embodiments, the inhibitor of CTLA4 is an anti-CTLA4 antibody. In some embodiments, the inhibitor of TIM3 is an anti-TIM3 antibody. In some embodiments, the inhibitor of LAG3 is an anti-LAG3 antibody. In some embodiments, the inhibitor of VISTA is an anti-VISTA antibody. In some embodiments, the inhibitor of BTLA is an anti-BTLA antibody. In some embodiments, the inhibitor of TIGIT is an anti-TIGIT antibody. In some embodiments, the inhibitor of LAIR1 is an anti-LAIR1 antibody. In some embodiments, the inhibitor of CD73 is an anti-CD73 antibody. In some embodiments, the inhibitor of CD160 is an anti-CD160 antibody. In some embodiments, the inhibitor of 2B4 is an anti-2B4 antibody. In some embodiments, the inhibitor of TGFRβ is an anti-TGFRβ antibody.

In some embodiments, the anti-PD-1 antibody is selected from pembrolizumab, nivolumab, cemiplimab, atezolizumab, dostralimab, durvalumab, avelumab, or any combination thereof. In some embodiments, the anti-PD-1 antibody is pembrolizumab. In some embodiments, the anti-PD-1 antibody is nivolumab. In some embodiments, the anti-PD-1 antibody is cemiplimab. In some embodiments, the anti-PD-1 antibody is atezolizumab. In some embodiments, the anti-PD-1 antibody is dostralimab. In some embodiments, the anti-PD-1 antibody is durvalumab. In some embodiments, the anti-PD-1 antibody is avelumab.

In some embodiments, the additional therapeutic is administered via a subcutaneous, intravenous, intramuscular, intraocular, intravitreal, intra-articular, intra-synovial, intrasternal, intrathecal, intrahepatic, intraperitoneal, intralesional, and intracranial injection, or infusion techniques.

In some embodiments, the additional therapeutic is administered at day 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days following implantation, injection, or delivery of the implantable constructs described herein. In some embodiments, the additional therapeutic is administered in a single dose. In some embodiments, the additional therapeutic is administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days following implantation, injection, or delivery of the implantable constructs described herein. In some embodiments, the additional therapeutic is administered every day following implantation, injection, or delivery of the implantable constructs described herein. In some embodiments, the additional therapeutic is administered every 2 days following implantation, injection, or delivery of the implantable constructs described herein. In some embodiments, the additional therapeutic is administered every 3 days following implantation, injection, or delivery of the implantable constructs described herein. In some embodiments, the additional therapeutic is administered every 4 days following implantation, injection, or delivery of the implantable constructs described herein. In some embodiments, the additional therapeutic is administered every 5 days following implantation, injection, or delivery of the implantable constructs described herein. In some embodiments, the additional therapeutic is administered every 6 days following implantation, injection, or delivery of the implantable constructs described herein. In some embodiments, the additional therapeutic is administered every 7 days following implantation, injection, or delivery of the implantable constructs described herein. In some embodiments, the additional therapeutic is administered every 8 days following implantation, injection, or delivery of the implantable constructs described herein. In some embodiments, the additional therapeutic is administered every 9 days following implantation, injection, or delivery of the implantable constructs described herein. In some embodiments, the additional therapeutic is administered every 10 days following implantation, injection, or delivery of the implantable constructs described herein. In some embodiments, the additional therapeutic is administered every 11 days following implantation, injection, or delivery of the implantable constructs described herein. In some embodiments, the additional therapeutic is administered every 12 days following implantation, injection, or delivery of the implantable constructs described herein. In some embodiments, the additional therapeutic is administered every 13 days following implantation, injection, or delivery of the implantable constructs described herein. In some embodiments, the additional therapeutic is administered every 14 days following implantation, injection, or delivery of the implantable constructs described herein. In some embodiments, the additional therapeutic is administered every 15 days following implantation, injection, or delivery of the implantable constructs described herein. In some embodiments, the additional therapeutic is administered every 16 days following implantation, injection, or delivery of the implantable constructs described herein. In some embodiments, the additional therapeutic is administered every 17 days following implantation, injection, or delivery of the implantable constructs described herein. In some embodiments, the additional therapeutic is administered every 18 days following implantation, injection, or delivery of the implantable constructs described herein. In some embodiments, the additional therapeutic is administered every 19 days following implantation, injection, or delivery of the implantable constructs described herein. In some embodiments, the additional therapeutic is administered every 20 days following implantation, injection, or delivery of the implantable constructs described herein. In some embodiments, the additional therapeutic is administered every 21 days following implantation, injection, or delivery of the implantable constructs described herein. In some embodiments, the additional therapeutic is administered every 22 days following implantation, injection, or delivery of the implantable constructs described herein. In some embodiments, the additional therapeutic is administered every 23 days following implantation, injection, or delivery of the implantable constructs described herein. In some embodiments, the additional therapeutic is administered every 24 days following implantation, injection, or delivery of the implantable constructs described herein. In some embodiments, the additional therapeutic is administered every 25 days following implantation, injection, or delivery of the implantable constructs described herein. In some embodiments, the additional therapeutic is administered every 26 days following implantation, injection, or delivery of the implantable constructs described herein. In some embodiments, the additional therapeutic is administered every 27 days following implantation, injection, or delivery of the implantable constructs described herein. In some embodiments, the additional therapeutic is administered every 28 days following implantation, injection, or delivery of the implantable constructs described herein. In some embodiments, the additional therapeutic is administered every 29 days following implantation, injection, or delivery of the implantable constructs described herein. In some embodiments, the additional therapeutic is administered every 30 days following implantation, injection, or delivery of the implantable constructs described herein. In some embodiments, the additional therapeutic is administered every 31 days following implantation, injection, or delivery of the implantable constructs described herein. In some embodiments, the additional therapeutic is administered at days 7, 10, 14, and 17 following implantation, injection, or delivery of the implantable constructs described herein.

In some embodiments, the additional therapeutic is administered as a single dose. In some embodiments, the additional therapeutic is administered as multiple doses.

### D. FEATURES OF IMPLANTABLE CONSTRUCTS

The implantable construct described herein may take any suitable shape or morphology. For example, an implantable construct may be a sphere, spheroid, tube, cord, string, ellipsoid, disk, cylinder, sheet, torus, cube, stadiumoid, cone, pyramid, triangle, rectangle, square, or rod. An implantable construct may comprise a curved or flat section. In an embodiment, an implantable construct may be prepared through the use of a mold, resulting in a custom shape.

The implantable construct may vary in size, depending, for example, on the use or site of implantation. For example, an implantable construct may have a mean diameter or size greater than 0.1 mm, e.g., greater than 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, or more. In an embodiment, an implantable construct may have a section or region with a mean diameter or size greater than 0.1 mm, e.g., greater than 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, or more. In an embodiment, an implantable construct may have a mean diameter or size less than 1 cm, e.g., less 50 mm, 40 mm, 30 mm, 20 mm, 10 mm, 7.5 mm, 5 mm, 2.5 mm, 1 mm, 0.5 mm, or smaller. In an embodiment, an implantable construct may have a section or region with a mean diameter or size less than 1 cm, e.g., less 50 mm, 40 mm, 30 mm, 20 mm, 10 mm, 7.5 mm, 5 mm, 2.5 mm, 1 mm, 0.5 mm, or smaller.

An implantable construct comprises at least one zone capable of preventing exposure of an enclosed antigenic or therapeutic agent from the outside milieu, e.g., a host effector cell or tissue. In an embodiment, the implantable construct comprises an inner zone (IZ). In an embodiment, the implantable construct comprises an outer zone (OZ). In an embodiment, either the inner zone (IZ) or outer zone (OZ) may be erodible or degradable. In an embodiment, the inner zone (IZ) is erodible or degradable. In an embodiment, the outer zone (OZ) is erodible or degradable. In an embodiment, the implantable construct comprises both an inner zone (IZ) and an outer zone (OZ), either of which may be erodible or degradable. In an embodiment, the implantable construct comprises both an inner zone (IZ) and an outer zone (OZ), wherein the outer zone is erodible or degradable. In an embodiment, the implantable construct comprises both an inner zone (IZ) and an outer zone (OZ), wherein the inner zone is erodible or degradable. The thickness of either of the zone, e.g., either the inner zone or outer zone, may be correlated with the length or duration of a “shielded” phase, in which the encapsulated antigenic or therapeutic agent is protected or shielded from the outside milieu, e.g., a host effector cell or tissue.

The zone (e.g., the inner zone or outer zone) of the implantable construct may comprise a degradable entity, e.g., an entity capable of degradation. A degradable entity may comprise an enzyme cleavage site, a photolabile site, a pH-sensitive site, or other labile region that can be eroded or comprised over time. In an embodiment, the degradable entity is preferentially degraded upon exposure to a first condition (e.g., exposure to a first milieu, e.g., a first pH or first enzyme) relative to a second condition (e.g., exposure to a second milieu, e.g., a second pH or second enzyme). In one embodiment, the degradable entity is degraded at least 2, 5, 10, 20, 30, 40, 50, 60, 70, 80, or 100 times faster upon exposure to a first condition relative to a second condition. In an embodiment, the degradable entity is an enzyme cleavage site, e.g., a proteolytic site. In an embodiment, the degradable entity is a polymer (e.g., a synthetic polymer or a naturally occurring polymer, e.g., a peptide or polysaccharide). In an embodiment, the degradable entity is a substrate for an endogenous host component, e.g., a degradative enzyme, e.g., a remodeling enzyme, e.g., a collagenase or metalloprotease. In an embodiment, the degradable entity comprises a cleavable linker or cleavable segment embedded in a polymer.

In an embodiment, an implantable construct comprises a pore or opening to permit passage of an object, such as a small molecule (e.g., nutrients or waste), a protein, or a nucleic acid. For example, a pore in or on an implantable construct may be greater than 0.1 nm and less than 10 m. In an embodiment, the implantable construct comprises a pore or opening with a size range of 0.1 μm to 10 μm, 0.1 μm to 9 μm, 0.1 μm to 8 μm, 0.1 μm to 7 μm, 0.1 μm to 6 μm, 0.1 μm to 5 μm, 0.1 μm to 4 μm, 0.1 μm to 3 μm, 0.1 μm to 2 μm.

An implantable construct described herein may comprise a chemical modification in or on any enclosed material. Exemplary chemical modifications include small molecules, peptides, proteins, nucleic acids, lipids, or oligosaccharides. The implantable construct may comprise at least 0.5%, 1%, 2%, 3%, 4%, 5%, 7.5%, 10%, 15%, 20%, 30%, 40%, 50%, 60%, 70%, 80% or more of a material that is chemically modified, e.g., with a chemical modification described herein. An implantable construct may be partially coated with a chemical modification, e.g., at least about 5%, 10%, 15%, 20%, 25%, 30%, 35%, 40%, 45%, 50%, 55%, 60%, 65%, 70%, 75%, 80%, 85%, 90%, 95%, 99%, or 99.9% coated with a chemical modification.

In an embodiment, the implantable construct is formulated such that the duration of release of the antigenic and/or therapeutic agent is tunable. For example, an implantable construct may be configured in a certain manner to release a specific amount of an antigenic or therapeutic agent over time, e.g., in a sustained or controlled manner. In an embodiment, the implantable construct comprises a zone (e.g., an inner zone or an outer zone) that is degradable, and this controls the duration of therapeutic release from the construct by gradually ceasing immunoprotection of encapsulated cells or causing gradual release of the antigenic agent. In an embodiment, the implantable construct is configured such that the level of release of an antigenic or therapeutic agent is sufficient to modulate the ratio of a host effector cell, e.g., a host T cell. In an embodiment, the implantable construct is configured such that the level of release of an antigenic or therapeutic agent is sufficient to activate a host cell (e.g., a host T effector cell or a host NK cell) or increase the level of certain host cells (e.g., host T effector cells or host NK cells). In an embodiment, the implantable construct is configured such that the level of release of an antigenic or therapeutic agent is not sufficient to activate a host regulator cell (e.g., a host T regulator cell) or increase the level of host regulator cells (e.g., host T regulator cells).

In some embodiments, the implantable construct comprises a zone that is targeted by the natural foreign body response (FBR) of a host or subject, e.g., over a period of time. In an embodiment, the implantable construct is coated with fibrotic overgrowth upon administration to a subject, e.g., over a period of time. Fibrotic overgrowth on the surface of the implantable construct may lead to a decrease in function of the implantable construct. For example, a decrease in function may comprise a reduction in the release of an antigenic or therapeutic agent over time, a decrease in pore size, or a decrease in the diffusion rate of oxygen and other key nutrients to the encapsulated cells, leading to cell death. In an embodiment, the rate of fibrotic overgrowth may be tuned to design a dosing regimen. For example, the fibrotic overgrowth on the surface of an implantable construct may result in a decrease in function of the implantable construct about 6 hours, 12 hours, 18 hours, 1 day, 2 days, 3 days, 4 days, 5 days, 6 days, 7 days, 8 days, 9 days, 10 days, 11 days, 12 days, 13 days, 2 weeks, 2.5 weeks, 3 weeks, 4 weeks, or 6 weeks after administration (e.g., injection or implantation) to a subject.

In some embodiments, the implantable construct is chemically modified with a specific density of modifications. The specific density of chemical modifications may be described as the average number of attached chemical modifications per given area. For example, the density of a chemical modification on or in an implantable construct may be 0.01, 0.1, 0.5, 1, 5, 10, 15, 20, 50, 75, 100, 200, 400, 500, 750, 1,000, 2,500, or 5,000 chemical modifications per square μm or square mm.

An implantable construct may be formulated or configured for implantation in any organ, tissue, cell, or part of a subject. For example, the implantable construct may be implanted or disposed into the peritoneal or the pleural cavity of a subject. An implantable construct may be implanted in or disposed on a tumor or other growth in a subject, or be implanted in or disposed about 0.1 mm, 0.5 mm, 1 mm, 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, 1 cm, 5, cm, 10 cm, or further from a tumor or other growth in a subject. An implantable construct may be configured for implantation, or implanted, or disposed on or in the skin, a mucosal surface, a body cavity, the central nervous system (e.g., the brain or spinal cord), an organ (e.g., the heart, eye, liver, kidney, spleen, lung, ovary, breast, uterus), the lymphatic system, vasculature, oral cavity, nasal cavity, gastrointestinal tract, bone, muscle, adipose tissue, skin, or other area.

An implantable construct may be formulated for use for any period of time. For example, an implantable construct may be used for 1 hour, 2 hours, 4 hours, 6 hours, 12 hours, 1 day, 36 hours, 2 days, 3 days, 4 days, 5 days, 6 days, 1 week, 2 weeks, 3 weeks, 4 weeks, 5 weeks, 6 weeks, 2 months, 3 months, 4 months, 5 months, 6 months, 1 year, or longer. An implantable construct can be configured for limited exposure (e.g., less than 2 days, e.g., less than 2 days, 1 day, 24 hours, 20 hours, 16 hours, 12 hours, 10 hours, 8 hours, 6 hours, 5 hours, 4 hours, 3 hours, 2 hours, 1 hour or less). A implantable construct can be configured for prolonged exposure (e.g., at least 2 days, 3 days, 4 days, 5 days, 6 days, 7 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 1.5 years, 2 years, 2.5 years, 3 years, 3.5 years, 4 years or more). An implantable construct can be configured for permanent exposure (e.g., at least 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 1.5 years, 2 years, 2.5 years, 3 years, 3.5 years, 4 years or more).

In some embodiments, the degradable zone comprises a polymeric hydrogel, such as but not limited to chitosan, cellulose, hyaluronic acid, or alginate. In some embodiments, the alginate is SLG20. In some embodiments, the SLG20 is about 0.1%-3% SLG20.

Accordingly, in some embodiments, a population of encapsulated cells comprising an oligonucleotide molecule encoding native human IL-2 are provided. In some embodiments, the oligonucleotide encoding native human IL-2 comprises a sequence of SEQ ID NO: 1. In some embodiments, the cell produces recombinant native human IL-2 protein. In some embodiments, the recombinant native human IL-2 protein expressed by the cells is formed from an amino acid sequence of (SEQ ID NO: 2). In some embodiments, the population of encapsulated cells produces about 1 to about 10, about 1 to about 5, or about 2 to about 4 PCD (picograms/cell/day) of native human IL-2. As provided herein, the cell can be any type of suitable cell, such as ARPE-19 cells.

In some embodiments, the cells in the encapsulated cells, which can also be referred to as the implantable construct, remain viable for at least 15, 20, 25, or 28 days. As used herein, the term, “viable” refers to a cell being able to produce IL-2 over this time period. In some embodiments, a viable cell is not a cell that is dividing. A cell can still be viable even if it is not dividing to expand the number of cells. In some embodiments, the encapsulated cells do not proliferate. Without being bound to any particular theory, the cells do not proliferate once encapsulated due to contact inhibition. In some embodiments, the encapsulated cells produced a sustained amount of IL-2 for at least 5, 10, 15, 20, or 24 hours In some embodiments, the encapsulated cells can produce a sustained amount of IL-2 for up to 30 days. Also provided for herein are pharmaceutical compositions comprising the encapsulated cells.

### E. METHODS OF TREATMENT

Described herein are methods of treatment or uses of encapsulated cells for the preparation of a pharmaceutical composition (or medicament) for the treatment of tumors or a disease.

In some embodiments, the disease is a proliferative disease. In an embodiment, the proliferative disease is cancer. A cancer may be an epithelial, mesenchymal, or hematological malignancy. A cancer includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). In an embodiment, the cancer is a solid tumor (e.g., carcinoid, carcinoma or sarcoma), a soft tissue tumor (e.g., a heme malignancy), or a metastatic lesion, e.g., a metastatic lesion of any of the cancers disclosed herein. In an embodiment, the cancer is a fibrotic or desmoplastic solid tumor. In some embodiments, the tumor is a mesothelioma tumor.

In some embodiments, the disease is a pleural disease or condition. Examples of pleural diseases or conditions include, but are not limited to: pleural cancer, pleural metastatic disease, pleurisy, lung infection, viral pneumonia, bacterial pneumonia, idiopathic pulmonary fibrosis, acute respiratory distress syndrome, pleural thickening, pleural pseudotumor, pleural plaque, extrapleural hematoma, Castleman disease, hemangioendothelioma, splenosis, paramalignang effusion, pleural effusion, pneumothorax, hemothorax, reactive pleuritis. In some embodiments, pleural cancer includes, but is not limited to lung cancer, metastases, mesothelioma, malignant mesothelioma, lymphoma, malignant fibrous tumor, sarcoma, askin tumor, extraskeletal osteosarcoma, malignant fibrous histiocytoma, solitary fibrous tumor, lipoma, mesothelial cyst, calcifying fibrous pseudotumor, primary effusion lymphoma.

In some embodiments, the cancer is mesothelioma. In some embodiments, the mesothelioma is a pleural mesothelioma, peritoneal mesothelioma, pericardial mesothelioma, testicular mesothelioma, epithelioid mesothelioma, sarcomatoid mesothelioma, biphasic mesothelioma, small cell mesothelioma, deciduoid mesothelioma, cystic and papillary mesothelioma, desmoplastic mesothelioma, adenomatoid mesothelioma, heterologous mesothelioma, well-defined papillary cell mesothelioma, or any combination thereof. In some embodiments, the mesothelioma is a pleural mesothelioma. In some embodiments, the mesothelioma is a peritoneal mesothelioma. In some embodiments, the mesothelioma is a pericardial mesothelioma. In some embodiments, the mesothelioma is a testicular mesothelioma. In some embodiments, the mesothelioma is a epithelioid mesothelioma. In some embodiments, the mesothelioma is a sarcomatoid mesothelioma. In some embodiments, the mesothelioma is a biphasic mesothelioma. In some embodiments, the mesothelioma is a small cell mesothelioma. In some embodiments, the mesothelioma is a deciduoid mesothelioma. In some embodiments, the mesothelioma is a cystic and papillary mesothelioma. In some embodiments, the mesothelioma is a desmoplastic mesothelioma. In some embodiments, the mesothelioma is a adenomatoid mesothelioma. In some embodiments, the mesothelioma is a heterologous mesothelioma. In some embodiments, the mesothelioma is a well-defined papillary cell mesothelioma.

In some embodiments, methods of treating a disease, in a subject, the method comprising implanting, or delivering to, the subject a pharmaceutical composition comprising a population of encapsulated cells comprising a heterologous oligonucleotide molecule encoding the native human cytokine and further administering an additional therapeutic are provided. In some embodiments, the native human cytokine is IL-1, IL-1α, IL-1β, IL-1RA, IL-4, IL-5, IL-6, IL-7, IL-8, IL-9, IL-10, IL-11, IL-12, IL-12a, IL-12b, IL-13, IL-14, IL-15, IL-16, IL-17, G-CSF, GM-CSF, IL-20, IL-23, IFN-α, IFN-β, IFN-γ, CD154, LT-β, CD70, CD153, CD178, TRAIL, TNF-α, TNF-β, SCF, M-CSF, MSP, 4-1BBL, LIF, OSM, or any combination thereof. In some embodiments, the native human cytokine is IL-2.

In some embodiments, methods of treating a disease, in a subject, the method comprising implanting, or delivering to, the subject, such as to the pleural cavity or IP space, of a pharmaceutical composition comprising a population of encapsulated cells comprising a heterologous oligonucleotide molecule encoding the native human IL-2 are provided. In some embodiments, the disease is as provided herein. In some embodiments, the disease is a cancer. In some embodiments, the disease is a pleural disease. In some embodiments, the pleural disease or condition is pleural cancer, pleural metastatic disease, pleurisy, lung infection, viral pneumonia, bacterial pneumonia, idiopathic pulmonary fibrosis, acute respiratory distress syndrome, pleural thickening, pleural pseudotumor, pleural plaque, extrapleural hematoma, Castleman disease, hemangioendothelioma, splenosis, paramalignang effusion, pleural effusion, pneumothorax, hemothorax, reactive pleuritis. In some embodiments, the pleural disease is a pleural cancer. In some embodiments, the pleural cancer is lung cancer, metastases, mesothelioma, malignant mesothelioma, lymphoma, malignant fibrous tumor, sarcoma, askin tumor, extraskeletal osteosarcoma, malignant fibrous histiocytoma, solitary fibrous tumor, lipoma, mesothelial cyst, calcifying fibrous pseudotumor, primary effusion lymphoma.

In some embodiments, methods of treating a disease, in a subject, the method comprising implanting, or delivering to, the subject a pharmaceutical composition comprising a population of encapsulated cells comprising a heterologous oligonucleotide molecule encoding the native human IL-2 and further administering an additional therapeutic are provided. In some embodiments, the disease is as provided herein. In some embodiments, the disease is a cancer. In some embodiments, the disease or condition is cancer. In some embodiments, the cancer is mesothelioma. In some embodiments, the mesothelioma is a pleural mesothelioma, peritoneal mesothelioma, pericardial mesothelioma, testicular mesothelioma, epithelioid mesothelioma, sarcomatoid mesothelioma, biphasic mesothelioma, small cell mesothelioma, deciduoid mesothelioma, cystic and papillary mesothelioma, desmoplastic mesothelioma, adenomatoid mesothelioma, heterologous mesothelioma, well-defined papillary cell mesothelioma, or any combination thereof. In some embodiments, the mesothelioma is a pleural mesothelioma. In some embodiments, the mesothelioma is a peritoneal mesothelioma. In some embodiments, the mesothelioma is a pericardial mesothelioma. In some embodiments, the mesothelioma is a testicular mesothelioma. In some embodiments, the mesothelioma is a epithelioid mesothelioma. In some embodiments, the mesothelioma is a sarcomatoid mesothelioma. In some embodiments, the mesothelioma is a biphasic mesothelioma. In some embodiments, the mesothelioma is a small cell mesothelioma. In some embodiments, the mesothelioma is a deciduoid mesothelioma. In some embodiments, the mesothelioma is a cystic and papillary mesothelioma. In some embodiments, the mesothelioma is a desmoplastic mesothelioma. In some embodiments, the mesothelioma is a adenomatoid mesothelioma. In some embodiments, the mesothelioma is a heterologous mesothelioma. In some embodiments, the mesothelioma is a well-defined papillary cell mesothelioma.

In some embodiments, methods of treating a pleural disease or condition, in a subject, the method comprising implanting, or delivering to, the pleural cavity a pharmaceutical composition comprising a population of encapsulated cells comprising a heterologous oligonucleotide molecule encoding the native human IL-2 are provided. In some embodiments, the pleural disease or condition is pleural cancer, pleural metastatic disease, pleurisy, lung infection, viral pneumonia, bacterial pneumonia, idiopathic pulmonary fibrosis, acute respiratory distress syndrome, pleural thickening, pleural pseudotumor, pleural plaque, extrapleural hematoma, Castleman disease, hemangioendothelioma, splenosis, paramalignang effusion, pleural effusion, pneumothorax, hemothorax, reactive pleuritis. In some embodiments, the pleural cancer is lung cancer, metastases, mesothelioma, malignant mesothelioma, lymphoma, malignant fibrous tumor, sarcoma, askin tumor, extraskeletal osteosarcoma, malignant fibrous histiocytoma, solitary fibrous tumor, lipoma, mesothelial cyst, calcifying fibrous pseudotumor, primary effusion lymphoma.

In some embodiments, methods of treating a pleural disease or condition, in a subject by generating memory immunity, the method comprising implanting, or delivering to, the pleural cavity a pharmaceutical composition comprising a population of encapsulated cells comprising a heterologous oligonucleotide molecule encoding the native human IL-2 are provided. In some embodiments, the pleural disease or condition is pleural cancer, pleural metastatic disease, pleurisy, lung infection, viral pneumonia, bacterial pneumonia, idiopathic pulmonary fibrosis, acute respiratory distress syndrome, pleural thickening, pleural pseudotumor, pleural plaque, extrapleural hematoma, Castleman disease, hemangioendothelioma, splenosis, paramalignang effusion, pleural effusion, pneumothorax, hemothorax, reactive pleuritis. In some embodiments, the pleural cancer is lung cancer, metastases, mesothelioma, malignant mesothelioma, lymphoma, malignant fibrous tumor, sarcoma, askin tumor, extraskeletal osteosarcoma, malignant fibrous histiocytoma, solitary fibrous tumor, lipoma, mesothelial cyst, calcifying fibrous pseudotumor, primary effusion lymphoma.

Accordingly, in some embodiments, methods of treating a cancer, in a subject, are provided. In some embodiments, the methods comprise implanting in the intraperitoneal space of the subject a pharmaceutical composition comprising a plurality of a population of encapsulated cells (e.g., a capsule) as provided for herein to treat the tumor. In some embodiments, the cancer is a pleural cancer. In some embodiments, the pleural cancer is lung cancer, metastases, mesothelioma, malignant mesothelioma, lymphoma, malignant fibrous tumor, sarcoma, askin tumor, extraskeletal osteosarcoma, malignant fibrous histiocytoma, solitary fibrous tumor, lipoma, mesothelial cyst, calcifying fibrous pseudotumor, primary effusion lymphoma. In some embodiments, the pleural cancer is mesothelioma.

In some embodiments, methods of providing systemic treatment to a subject with cancer, the method comprising implanting in the pleural cavity of the subject a pharmaceutical composition comprising a population of encapsulated cells comprising a heterologous oligonucleotide molecule encoding the native human IL-2, whereby the pharmaceutical composition stimulates the activation of immune cells in the pleural cavity and the activated immune cells migrate to a region of the subject that is distal to the pleural cavity to treat the cancer systemically in the subject are provided.

In some embodiments, methods of providing systemic treatment to a subject with cancer are provided. In some embodiments, the methods comprise implanting in the pleural cavity of the subject a pharmaceutical composition comprising a population of encapsulated cells comprising a heterologous oligonucleotide molecule encoding the native human IL-2, whereby the pharmaceutical composition activates immune cells and the activated immune cells migrate out of the pleural cavity to treat the cancer in the subject. In some embodiments, the pharmaceutical composition activates immune cells in the pleural cavity. In some embodiments, the activated immune cells migrate out of (away from) the pleural cavity to treat the cancer in the subject at a site that is not in the pleural cavity. In some embodiments, the activated immune cells migrate out of (away from) the pleural cavity to treat the cancer in the subject at a site that is distal from the pleural cavity. In some embodiments, the site is another organ or tissue, such as pancreas, breast, brain, lungs, bone, or as otherwise provided for herein. Without being bound to any particular theory, because the compositions provided for herein can deliver native IL-2 that is produced by the cells in a localized space, the negative effects of IL-2 that has been previously delivered systematically can be reduced or eliminated. Thus, the positive, or therapeutic effect, of IL-2 can be delivered to the subject without producing, or by reducing, the systemic side effects seen with the systemic administration of IL-2. Accordingly, in some embodiments, the subject has fewer side effects as compared to a subject that is administered a pharmaceutical composition systemically, such as intravenously administered IL-2 or intravenously administered compositions provided for herein. In some embodiments, the activated immune cells are CD8 positive effector T cells. In some embodiments, the effector T cells are selectively activated and expanded at least 1, 2, 3, 4, or 5 times as compared to Tregs in the pleural cavity. In some embodiments, the effector T cells are selectively activated and expanded at least 1, 2, 3, 4, or 5 times as compared to Tregs systemically.

In some embodiments, methods of selectively activating CD4 and CD8 positive T cells are provided. Without being bound by any particular theory, the CD4 and CD8 positive T cells are activated and trigger an immune response against the tumor. This can be initiated or enhanced by the secretion of native human cytokine, such as but not limited to, IL-2 in the peritoneal or pleural cavity from the encapsulated cells that are provided for herein. In some embodiments, the methods comprise implanting a pharmaceutical composition comprising a population of encapsulated cells and an additional therapeutic as provided for herein. In some embodiments, the methods comprise implanting into the peritoneal or pleural cavity a pharmaceutical composition comprising a population of encapsulated cells and an additional therapeutic as provided for herein.

In some embodiments, methods of increasing CD4 and CD8 positive effector T cells are provided. In some embodiments, the methods comprise implanting a pharmaceutical composition comprising a population of encapsulated cells and an additional therapeutic as provided for herein. In some embodiments, the methods comprise implanting into the peritoneal or pleural cavity a pharmaceutical composition comprising a population of encapsulated cells and an additional therapeutic as provided for herein.

In some embodiments, methods of shifting macrophage phenotype are provided. In some embodiments, methods of shifting M2-like macrophages to M1-like macrophages are provided. In some embodiments, methods of reducing M2-like macrophages and increasing M1-like macrophages are provided. In some embodiments, the methods comprise implanting a pharmaceutical composition comprising a population of encapsulated cells and an additional therapeutic as provided for herein. In some embodiments, the methods comprise implanting into the peritoneal or pleural cavity a pharmaceutical composition comprising a population of encapsulated cells and an additional therapeutic as provided for herein.

In some embodiments, methods of increasing dendritic cells are provided. In some embodiments, the dendritic cells are MHC II+ dendritic cells. In some embodiments, methods of shifting macrophage phenotype and increasing MHC II+ dendritic cells are provided. In some embodiments, methods of shifting M2-like macrophages to M1-like macrophages and increasing MHC II+ dendritic cells are provided. In some embodiments, methods of reducing M-2 like macrophages, and increasing M1-like macrophages and MHC II+ dendritic cells are provided. In some embodiments, the methods comprise implanting a pharmaceutical composition comprising a population of encapsulated cells and an additional therapeutic as provided for herein. In some embodiments, the methods comprise implanting into the peritoneal or pleural cavity a pharmaceutical composition comprising a population of encapsulated cells and an additional therapeutic as provided for herein.

In some embodiments, methods of treating a disease or condition, in a subject, the method comprising implanting, or delivering to, the subject a pharmaceutical composition comprising a population of encapsulated cells comprising a heterologous oligonucleotide molecule encoding the native human cytokine and further administering an additional therapeutic are provided. In some embodiments, the native human cytokine is IL-1, IL-1α, IL-1β, TL-1RA, IL-4, IL-5, IL-6, IL-7, IL-8, IL-9, IL-10, IL-11, IL-12, IL-12a, IL-12b, IL-13, IL-14, IL-15, IL-16, IL-17, G-CSF, GM-CSF, IL-20, IL-23, IFN-α, IFN-β, IFN-γ, CD154, LT-β, CD70, CD153, CD178, TRAIL, TNF-α, TNF-β, SCF, M-CSF, MSP, 4-1BBL, LIF, OSM, or any combination thereof. In some embodiments, the native human cytokine is IL-2.

In some embodiments, methods of treating a disease or condition, in a subject, the method comprising implanting, or delivering to, the subject a pharmaceutical composition comprising a population of encapsulated cells comprising a heterologous oligonucleotide molecule encoding the native human IL-2 and further administering an additional therapeutic are provided. In some embodiments, the disease or condition is pleural cancer. In some embodiments, the cancer is mesothelioma. In some embodiments, the mesothelioma is a pleural mesothelioma, peritoneal mesothelioma, pericardial mesothelioma, testicular mesothelioma, epithelioid mesothelioma, sarcomatoid mesothelioma, biphasic mesothelioma, small cell mesothelioma, deciduoid mesothelioma, cystic and papillary mesothelioma, desmoplastic mesothelioma, adenomatoid mesothelioma, heterologous mesothelioma, well-defined papillary cell mesothelioma, or any combination thereof. In some embodiments, the mesothelioma is a pleural mesothelioma. In some embodiments, the mesothelioma is a peritoneal mesothelioma. In some embodiments, the mesothelioma is a pericardial mesothelioma. In some embodiments, the mesothelioma is a testicular mesothelioma. In some embodiments, the mesothelioma is a epithelioid mesothelioma. In some embodiments, the mesothelioma is a sarcomatoid mesothelioma. In some embodiments, the mesothelioma is a biphasic mesothelioma. In some embodiments, the mesothelioma is a small cell mesothelioma. In some embodiments, the mesothelioma is a deciduoid mesothelioma. In some embodiments, the mesothelioma is a cystic and papillary mesothelioma. In some embodiments, the mesothelioma is a desmoplastic mesothelioma. In some embodiments, the mesothelioma is a adenomatoid mesothelioma. In some embodiments, the mesothelioma is a heterologous mesothelioma. In some embodiments, the mesothelioma is a well-defined papillary cell mesothelioma.

Accordingly, in some embodiments, methods of treating a cancer, in a subject, are provided. In some embodiments, the methods comprise implanting in the peritoneal or pleural space of the subject a pharmaceutical composition comprising a plurality of a population of encapsulated cells (e.g., a capsule) as provided for herein to treat the tumor. In some embodiments, the methods further comprise administering an additional therapeutic such as those provided herein. In some embodiments, the cancer is a mesothelioma. In some embodiments, the mesothelioma is a pleural mesothelioma, peritoneal mesothelioma, pericardial mesothelioma, testicular mesothelioma, epithelioid mesothelioma, sarcomatoid mesothelioma, biphasic mesothelioma, small cell mesothelioma, deciduoid mesothelioma, cystic and papillary mesothelioma, desmoplastic mesothelioma, adenomatoid mesothelioma, heterologous mesothelioma, well-defined papillary cell mesothelioma, or any combination thereof. In some embodiments, the mesothelioma is a pleural mesothelioma. In some embodiments, the mesothelioma is a peritoneal mesothelioma. In some embodiments, the mesothelioma is a pericardial mesothelioma. In some embodiments, the mesothelioma is a testicular mesothelioma. In some embodiments, the mesothelioma is a epithelioid mesothelioma. In some embodiments, the mesothelioma is a sarcomatoid mesothelioma. In some embodiments, the mesothelioma is a biphasic mesothelioma. In some embodiments, the mesothelioma is a small cell mesothelioma. In some embodiments, the mesothelioma is a deciduoid mesothelioma. In some embodiments, the mesothelioma is a cystic and papillary mesothelioma. In some embodiments, the mesothelioma is a desmoplastic mesothelioma. In some embodiments, the mesothelioma is an adenomatoid mesothelioma. In some embodiments, the mesothelioma is a heterologous mesothelioma. In some embodiments, the mesothelioma is a well-defined papillary cell mesothelioma.

In some embodiments, methods of providing systemic treatment to a subject with cancer, the method comprising implanting in a cavity of the subject a pharmaceutical composition comprising a population of encapsulated cells comprising a heterologous oligonucleotide molecule encoding the native human cytokine; and administering an immunomodulatory agent; whereby the pharmaceutical composition stimulates the activation of immune cells in the cavity and the activated immune cells migrate to a region of the subject that is distal to the cavity to treat the cancer systemically in the subject, are provided. In some embodiments, the native human cytokine is IL-1, IL-1α, IL-1β, IL-1RA, IL-4, IL-5, IL-6, IL-7, IL-8, IL-9, IL-10, IL-11, IL-12, IL-12a, IL-12b, IL-13, IL-14, IL-15, IL-16, IL-17, G-CSF, GM-CSF, IL-20, IL-23, IFN-α, IFN-β, IFN-γ, CD154, LT-β, CD70, CD153, CD178, TRAIL, TNF-α, TNF-β, SCF, M-CSF, MSP, 4-1BBL, LIF, OSM, or any combination thereof. In some embodiments, the native human cytokine is IL-2. In some embodiments, methods of providing systemic treatment to a subject with cancer, the method comprising implanting in a cavity of the subject a pharmaceutical composition comprising a population of encapsulated cells comprising a heterologous oligonucleotide molecule encoding the native human IL-2; and administering an immunomodulatory agent; whereby the pharmaceutical composition stimulates the activation of immune cells in the cavity and the activated immune cells migrate to a region of the subject that is distal to the cavity to treat the cancer systemically in the subject, are provided.

In some embodiments, methods of providing systemic treatment to a subject with cancer, the method comprising implanting in a cavity of the subject a pharmaceutical composition comprising a population of encapsulated cells comprising a heterologous oligonucleotide molecule encoding the native human cytokine; and administering an immunomodulatory agent; whereby the pharmaceutical composition activates immune cells and the activated immune cells migrate out of the cavity to treat the cancer in the subject, are provided. In some embodiments, the native human cytokine is IL-1, IL-1α, IL-1β, IL-1RA, IL-4, IL-5, IL-6, IL-7, IL-8, IL-9, IL-10, IL-11, IL-12, IL-12a, IL-12b, IL-13, IL-14, IL-15, IL-16, IL-17, G-CSF, GM-CSF, IL-20, IL-23, IFN-α, IFN-β, IFN-γ, CD154, LT-β, CD70, CD153, CD178, TRAIL, TNF-α, TNF-β, SCF, M-CSF, MSP, 4-1BBL, LIF, OSM, or any combination thereof. In some embodiments, the native human cytokine is IL-2. In some embodiments, methods of providing systemic treatment to a subject with cancer, the method comprising implanting in a cavity of the subject a pharmaceutical composition comprising a population of encapsulated cells comprising a heterologous oligonucleotide molecule encoding the native human IL-2; and administering an immunomodulatory agent; whereby the pharmaceutical composition activates immune cells and the activated immune cells migrate out of the cavity to treat the cancer in the subject, are provided.

In some embodiments, the cavity is a peritoneal cavity and/or pleural cavity. In some embodiments, the cavity is a peritoneal cavity. In some embodiments, the cavity is a pleural cavity. In some embodiments, the cavity is a peritoneal cavity and pleural cavity.

In some embodiments, methods of providing systemic treatment to a subject with cancer, the method comprising implanting in the pleural cavity of the subject a pharmaceutical composition comprising a population of encapsulated cells comprising a heterologous oligonucleotide molecule encoding the native human cytokine, and further administering an additional therapeutic such as those provided herein, whereby the pharmaceutical composition stimulates the activation of immune cells in the pleural cavity and the activated immune cells migrate to a region of the subject that is distal to the pleural cavity to treat the cancer systemically in the subject are provided. In some embodiments, the native human cytokine is IL-1, IL-1α, IL-1β, IL-1RA, IL-4, IL-5, IL-6, IL-7, IL-8, IL-9, IL-10, IL-11, IL-12, IL-12a, IL-12b, IL-13, IL-14, IL-15, IL-16, IL-17, G-CSF, GM-CSF, IL-20, IL-23, IFN-α, IFN-β, IFN-γ, CD154, LT-β, CD70, CD153, CD178, TRAIL, TNF-α, TNF-β, SCF, M-CSF, MSP, 4-1BBL, LIF, OSM, or any combination thereof. In some embodiments, the native human cytokine is IL-2. In some embodiments, methods of providing systemic treatment to a subject with cancer, the method comprising implanting in the pleural cavity of the subject a pharmaceutical composition comprising a population of encapsulated cells comprising a heterologous oligonucleotide molecule encoding the native human IL-2, and further administering an additional therapeutic such as those provided herein, whereby the pharmaceutical composition stimulates the activation of immune cells in the pleural cavity and the activated immune cells migrate to a region of the subject that is distal to the pleural cavity to treat the cancer systemically in the subject are provided.

In some embodiments, methods of providing systemic treatment to a subject with cancer, the method comprising implanting in the peritoneal cavity of the subject a pharmaceutical composition comprising a population of encapsulated cells comprising a heterologous oligonucleotide molecule encoding the native human cytokine, and further administering an additional therapeutic such as those provided herein, whereby the pharmaceutical composition stimulates the activation of immune cells in the peritoneal cavity and the activated immune cells migrate to a region of the subject that is distal to the peritoneal cavity to treat the cancer systemically in the subject are provided. In some embodiments, the native human cytokine is IL-1, IL-1α, IL-1β, IL-1RA, IL-4, IL-5, IL-6, IL-7, IL-8, IL-9, IL-10, IL-11, IL-12, IL-12a, IL-12b, IL-13, IL-14, IL-15, IL-16, IL-17, G-CSF, GM-CSF, IL-20, IL-23, IFN-α, IFN-β, IFN-γ, CD154, LT-β, CD70, CD153, CD178, TRAIL, TNF-α, TNF-β, SCF, M-CSF, MSP, 4-1BBL, LIF, OSM, or any combination thereof. In some embodiments, the native human cytokine is IL-2. In some embodiments, methods of providing systemic treatment to a subject with cancer, the method comprising implanting in the peritoneal cavity of the subject a pharmaceutical composition comprising a population of encapsulated cells comprising a heterologous oligonucleotide molecule encoding the native human IL-2, and further administering an additional therapeutic such as those provided herein, whereby the pharmaceutical composition stimulates the activation of immune cells in the peritoneal cavity and the activated immune cells migrate to a region of the subject that is distal to the peritoneal cavity to treat the cancer systemically in the subject are provided.

In some embodiments, methods of providing systemic treatment to a subject with cancer are provided. In some embodiments, the methods comprise implanting in the peritoneal cavity of the subject a pharmaceutical composition comprising a population of encapsulated cells comprising a heterologous oligonucleotide molecule encoding the native human IL-2, and further administering an additional therapeutic such as those provided herein, whereby the pharmaceutical composition activates immune cells and the activated immune cells migrate out of the peritoneal cavity to treat the cancer in the subject. In some embodiments, the methods comprise implanting in the pleural cavity of the subject a pharmaceutical composition comprising a population of encapsulated cells comprising a heterologous oligonucleotide molecule encoding the native human IL-2, and further administering an additional therapeutic such as those provided herein, whereby the pharmaceutical composition activates immune cells and the activated immune cells migrate out of the pleural cavity to treat the cancer in the subject. In some embodiments, the pharmaceutical composition activates immune cells in the peritoneal cavity. In some embodiments, the pharmaceutical composition activates immune cells in the pleural cavity. In some embodiments, the activated immune cells migrate out of (away from) the peritoneal cavity to treat the cancer in the subject at a site that is not in the peritoneal cavity. In some embodiments, the activated immune cells migrate out of (away from) the pleural cavity to treat the cancer in the subject at a site that is not in the pleural cavity. In some embodiments, the activated immune cells migrate out of (away from) the peritoneal cavity to treat the cancer in the subject at a site that is distal from the peritoneal cavity. In some embodiments, the activated immune cells migrate out of (away from) the pleural cavity to treat the cancer in the subject at a site that is distal from the pleural cavity. In some embodiments, the site is another organ or tissue, such as pancreas, breast, brain, lungs, bone, or as otherwise provided for herein. Without being bound to any particular theory, because the compositions provided for herein can deliver native IL-2 that is produced by the cells in a localized space, the negative effects of IL-2 that has been previously delivered systematically can be reduced or eliminated. Thus, the positive, or therapeutic effect, of IL-2 can be delivered to the subject without producing, or by reducing, the systemic side effects seen with the systemic administration of IL-2. Accordingly, in some embodiments, the subject has fewer side effects as compared to a subject that is administered a pharmaceutical composition systemically, such as intravenously administered IL-2 or intravenously administered compositions provided for herein. In some embodiments, the activated immune cells are CD4 and CD8 positive T cells.

As described herein, the encapsulated cells producing the recombinant native human IL-2 can be used to create memory immunity against a tumor. In some embodiments, the encapsulated cells producing the recombinant native human IL-2 can be used to create memory immunity against a cancer. Thus, in some embodiments, the methods provided herein can be used to prevent or reduce the probability of a tumor recurring either at the initial site of the tumor or a site that is distal to the origin of the tumor. In some embodiments, the tumor is a mesothelioma tumor. In some embodiments, the mesothelioma tumor is a mesothelioma cancer as provided herein. In some embodiments, the methods of treating a tumor by generating (inducing) memory immunity comprise implanting a pharmaceutical composition comprising a population of encapsulated cells as provided for herein.

In some embodiments, methods of selectively activating CD8 and/or CD4-positive effector T cells are provided. Without being bound by any particular theory, the CD8-positive and/or CD4-positive effector cells are activated and trigger an immune response against the tumor. This can be initiated or enhanced by the secretion of native human IL-2 in the pleural cavity from the encapsulated cells that are provided for herein. In some embodiments, the methods comprise implanting a pharmaceutical composition comprising a population of encapsulated cells as provided for herein. In some embodiments, the methods comprise implanting into the pleural cavity a pharmaceutical composition comprising a population of encapsulated cells as provided for herein.

In some embodiments, the effector T cells are selectively activated and expanded as compared to Tregs (CD4+CD25+FOXp3+). In some embodiments, the effector T cells (e.g., CD8 and/or CD4 positive T cells) are selectively activated and expanded at least 1, 2, 3, 4, or 5 times as compared to Tregs.

Exemplary cancers that can be treated by the methods provided for herein include, but are not limited to, carcinoma, lymphoma, blastoma, sarcoma, and leukemia or lymphoid malignancies. In an embodiment, the cancer affects a system of the body, e.g., the nervous system (e.g., peripheral nervous system (PNS) or central nervous system (CNS)), vascular system, skeletal system, respiratory system, endocrine system, lymph system, reproductive system, or gastrointestinal tract. In some embodiments, cancer affects a part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Other examples of cancers include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

In some embodiments, the implantable construct (encapsulated cells) described herein may be used to treat mesothelioma. Mesothelioma is a tumor that occurs in the mesothelium that covers the surface of the pleura, peritoneum and pericardium that respectively envelop the organs of the chest cavity such as the lungs and heart, and abdominal organs such as the digestive tract and liver. Without wishing to be bound to a particular theory, in the case of diffuse pleural mesothelioma, chest pain is caused by invasion of the intercostals nerves on the side of the chest wall pleura, and respiratory and circulatory disorders may occur due to tumor growth and accumulation of pleural fluid in the pleura on the organ side (Takagi, Journal of Clinical and Experimental Medicine, (March Supplement), “Respiratory Diseases”, pp. 469-472, 1999). Few effective treatments exist and 5-year mortality is approximately 90%. Malignant mesothelioma (MM) affects the organs that are lined by the mesothelium, including the organs of the chest (pleura) and abdomen (peritoneum). The combination of pemetrexed and cisplatin treatment was determined to be the first-line chemotherapy for malignant mesothelioma. More recently, the anti-angiogenic agent Bevacizumab increased survival by 2 months when added to pemetrexed/cisplatin (Zalcman G, Mazieres J, Margery J, Greillier L, Audigier-Valette C, Moro-Sibilot D, et al. Bevacizumab for newly diagnosed pleural mesothelioma in the Mesothelioma Avastin Cisplatin Pemetrexed Study (MAPS): a randomised, controlled, open-label, phase 3 trial. Lancet 2016; 387(10026):1405-14 doi 10.1016/S0140-6736(15)01238-6). Radiation is limited by the large size of its required field, and it is ineffective as a primary treatment (Boutin C, Rey F, Viallat J R. Prevention of malignant seeding after invasive diagnostic procedures in patients with pleural mesothelioma. A randomized trial of local radiotherapy. Chest 1995; 108(3):754-8; Rusch V W, Rosenzweig K, Venkatraman E, Leon L, Raben A, Harrison L, et al. A phase II trial of surgical resection and adjuvant high dose hemithoracic radiation for malignant pleural mesothelioma. The Journal of thoracic and cardiovascular surgery 2001; 122(4):788-95 doi 10.1067/mtc.2001.116560; Gomez D R, Hong D S, Allen P K, Welsh J S, Mehran R J, Tsao A S, et al. Patterns of failure, toxicity, and survival after extrapleural pneumonectomy and hemithoracic intensity-modulated radiation therapy for malignant pleural mesothelioma. Journal of thoracic oncology: official publication of the International Association for the Study of Lung Cancer 2013; 8(2):238-45 doi 10.1097/JTO.0b013e31827740f0; Rice D C, Stevens C W, Correa A M, Vaporciyan A A, Tsao A, Forster K M, et al. Outcomes after extrapleural pneumonectomy and intensity-modulated radiation therapy for malignant pleural mesothelioma. The Annals of thoracic surgery 2007; 84(5):1685-92; discussion 92-3 doi 10.1016/j.athoracsur.2007.04.076). In recent trials, immune checkpoint inhibitors such as nivolumab or pembrolizumab have shown encouraging clinical activity and good tolerability in patients with advanced malignant pleural mesothelioma (Scherpereel A, Mazieres J, Greillier L, Lantuejoul S, Do P, Bylicki O, et al. Nivolumab or nivolumab plus ipilimumab in patients with relapsed malignant pleural mesothelioma (IFCT-1501 MAPS2): a multicentre, open-label, randomised, non-comparative, phase 2 trial. Lancet Oncol 2019; 20(2):239-53 doi 10.1016/S1470-2045(18)30765-4; Baas P, Scherpereel A, Nowak A K, Fujimoto N, Peters S, Tsao A S, et al. First-line nivolumab plus ipilimumab in unresectable malignant pleural mesothelioma (CheckMate 743): a multicentre, randomised, open-label, phase 3 trial. Lancet 2021; 397(10272):375-86 doi 10.1016/S0140-6736(20)32714-8). Objective response rates (ORR) ranged from 15 to 21%, and rates of stable disease (SD) ranged from 33% to 56%, equating to 53% of patients experiencing durable clinical benefit (DCB; i.e., ORR+SD) (Alley E W, Lopez J, Santoro A, Morosky A, Saraf S, Piperdi B, et al. Clinical safety and activity of pembrolizumab in patients with malignant pleural mesothelioma (KEYNOTE-028): preliminary results from a non-randomised, open-label, phase 1b trial. The Lancet Oncology 2017; 18(5):623-30 doi 10.1016/S1470-2045(17)30169-9; Quispel-Janssen J, van der Noort V, de Vries J F, Zimmerman M, Lalezari F, Thunnissen E, et al. Programmed Death 1 Blockade With Nivolumab in Patients With Recurrent Malignant Pleural Mesothelioma. J Thorac Oncol 2018; 13(10):1569-76 doi 10.1016/j.jtho.2018.05.038; Metaxas Y, Rivalland G, Mauti L A, Klingbiel D, Kao S, Schmid S, et al. Pembrolizumab as Palliative Immunotherapy in Malignant Pleural Mesothelioma. Journal of thoracic oncology: official publication of the International Association for the Study of Lung Cancer 2018; 13(11):1784-91 doi 10.1016/j.jtho.2018.08.007; Kindler H K T, Carol Tan Y. Rose B, Ahmad M, Straus C, Sargis R, Seiwert T. OA13.02 Phase II Trial of Pembrolizumab in Patients with Malignant Mesothelioma (MM): Interim Analysis. journal of Thoracic Oncology 2017; 12:S293-S4). These results led to its recent approval by the FDA as the first line of defense for malignant pleural mesothelioma (Janes S M, Alrifai D, Fennell D A. Perspectives on the Treatment of Malignant Pleural Mesothelioma. N. Engl. J. Med. 2021; 385(13):1207-18 doi 10.1056/NEJMra1912719; Wright K. FDA Approves Nivolumab Plus Ipilimumab for Previously Untreated Unresectable Malignant Pleural Mesothelioma. Oncology (Williston Park) 2020; 34(11):502-3 doi 10.46883/ONC.2020.3411.0502). Despite promising clinical results, optimal and safe delivery remains a challenge (Johnson D B, Balko J M, Compton M L, Chalkias S, Gorham J, Xu Y, et al. Fulminant Myocarditis with Combination Immune Checkpoint Blockade. N. Engl. J. Med. 2016; 375(18):1749-55 doi 10.1056/NEJMoa1609214). Side effects of ICIs are predominantly immunologic, and immune adverse events (iAEs) occur in 74% of patients receiving PD-1 inhibitors, 14% of which are grade III-IV iAEs (Dougan M, Pietropaolo M. Time to dissect the autoimmune etiology of cancer antibody immunotherapy. J. Clin. Invest. 2020; 130(1):51-61 doi 10.1172/JCI131194). Local drug delivery can reduce toxicity by confining the immunostimulatory effects to the tumor microenvironment, highlighting a strong rationale for developing such approaches (Riley R S, June C H, Langer R, Mitchell M J. Delivery technologies for cancer immunotherapy. Nat. Rev. Drug Discov. 2019; 18(3):175-96 doi 10.1038/s41573-018-0006-z).

In some embodiments, the mesothelioma is a pleural mesothelioma, a malignant pleural mesothelioma, diffuse pleural mesothelioma, peritoneal mesothelioma, pericardial mesothelioma, testicular mesothelioma, epithelioid mesothelioma, sarcomatoid mesothelioma, biphasic mesothelioma, small cell mesothelioma, deciduoid mesothelioma, cystic and papillary mesothelioma, desmoplastic mesothelioma, adenomatoid mesothelioma, heterologous mesothelioma, well-defined papillary cell mesothelioma, or any combination thereof. In some embodiments, methods of treating mesothelioma are provided. In some embodiments, methods of treating pleural mesothelioma are provided. In some embodiments, methods of treating malignant pleural mesothelioma are provided. In some embodiments, methods of treating a peritoneal mesothelioma are provided. In some embodiments, methods of treating a pericardial mesothelioma are provided. In some embodiments, methods of treating a testicular mesothelioma are provided. In some embodiments, methods of treating an epithelioid mesothelioma are provided. In some embodiments, methods of treating a sarcomatoid mesothelioma are provided. In some embodiments, methods of treating a biphasic mesothelioma are provided. In some embodiments, methods of treating a small cell mesothelioma are provided. In some embodiments, methods of treating a deciduoid mesothelioma are provided. In some embodiments, methods of treating a cystic and papillary mesothelioma are provided. In some embodiments, methods of treating a desmoplastic mesothelioma are provided. In some embodiments, methods of treating an adenomatoid mesothelioma are provided. In some embodiments, methods of treating a heterologous mesothelioma are provided. In some embodiments, methods of treating a well-defined papillary cell mesothelioma are provided.

In some embodiments, methods of treating mesothelioma comprise administration of the implantable construct as provided herein. In some embodiments, methods of treating mesothelioma comprise administration of the implantable construct as provided herein to the subject in need thereof. In some embodiments, methods of treating mesothelioma comprise administration of the implantable construct as provided herein into the pleural space. In some embodiments, methods of treating mesothelioma comprise administration of the implantable construct as provided herein into the pleural space of a subject in need thereof.

In some embodiments, methods of treating mesothelioma comprise administration of the implantable construct and an additional therapeutic as provided herein. In some embodiments, methods of treating mesothelioma comprise administration of the implantable construct and an additional therapeutic as provided herein to the subject in need thereof. In some embodiments, methods of treating mesothelioma comprise administration of the implantable construct and an additional therapeutic as provided herein into the pleural space. In some embodiments, methods of treating mesothelioma comprise administration of the implantable construct and an additional therapeutic as provided herein into the peritoneal space. In some embodiments, methods of treating mesothelioma comprise administration of the implantable construct and an additional therapeutic as provided herein into the pleural space of a subject in need thereof. In some embodiments, methods of treating mesothelioma comprise administration of the implantable construct and an additional therapeutic as provided herein into the peritoneal space of a subject in need thereof.

In some embodiments, a method of treating a mesothelioma, in a subject, the method comprising implanting, or delivering to, the pleural cavity a pharmaceutical composition comprising a population of encapsulated cells comprising a heterologous oligonucleotide molecule encoding the native human IL-2 is provided. In some embodiments, the mesothelioma is a pleural mesothelioma, malignant pleural mesothelioma, diffuse pleural mesothelioma, peritoneal mesothelioma, pericardial mesothelioma, testicular mesothelioma, epithelioid mesothelioma, sarcomatoid mesothelioma, biphasic mesothelioma, small cell mesothelioma, deciduoid mesothelioma, cystic and papillary mesothelioma, desmoplastic mesothelioma, adenomatoid mesothelioma, heterologous mesothelioma, well-defined papillary cell mesothelioma, or any combination thereof. In some embodiments, the mesothelioma is a pleural mesothelioma. In some embodiments, the mesothelioma is a malignant pleural mesothelioma. In some embodiments, the mesothelioma is a diffuse pleural mesothelioma.

In some embodiments, the concentration of native human IL-2 in the pleural fluid at day 1 post implantation is at least 3000 μg/ml, 4000 μg/ml, 5000 μg/ml, 10000 μg/ml, 15000 μg/ml, or 20000 μg/ml. In some embodiments, the concentration of the recombinant native human IL-2 in the blood of the subject is substantially undetectable 1 day after implantation. In some embodiments, the concentration of the recombinant native human IL-2 in the pleural fluid of the subject is substantially undetectable 30 days after implantation. In some embodiments, the concentration of the recombinant native human IL-2 in the blood of the subject is substantially undetectable 1 day after implantation and is at least 3000 μg/ml, 4000 μg/ml, 5000 μg/ml, 10000 μg/ml, 15000 μg/ml, or 20000 μg/ml in the pleural fluid of the subject. In some embodiments, the concentration of the native human IL-2 is determined at, or at least, 1 day, 2 day, 3 day, 4 day, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days post implantation into the pleural cavity. In some embodiments, the recombinant native IL-2 protein is detectable in the pleural fluid of the subject at least 1 day post implantation.

In some embodiments, a method of treating a mesothelioma, in a subject by generating memory immunity, the method comprising implanting, or delivering to, the pleural cavity a pharmaceutical composition comprising a population of encapsulated cells comprising a heterologous oligonucleotide molecule encoding the native human IL-2 is provided. In some embodiments, the mesothelioma is a pleural mesothelioma, a malignant pleural mesothelioma, or a diffuse pleural mesothelioma. In some embodiments, the mesothelioma is a pleural mesothelioma. In some embodiments, the mesothelioma is a malignant pleural mesothelioma.

In some embodiments, the mesothelioma is a diffuse pleural mesothelioma. In some embodiments, the implanting, or delivering, comprises implanting the pharmaceutical composition comprising a plurality of the population of encapsulated cells (e.g., a capsule) as provided herein. In some embodiments, the implanting, or delivering, is achieved using methods and/or devices provided herein. In some embodiments, the concentration of native human IL-2 in the pleural fluid at day 1 post implantation is at least 3000 μg/ml, 4000 μg/ml, 5000 μg/ml, 10000 μg/ml, 15000 μg/ml, or 20000 μg/ml. In some embodiments, the concentration of the recombinant native human IL-2 in the blood of the subject is substantially undetectable 1 day after implantation. In some embodiments, the concentration of the recombinant native human IL-2 in the pleural fluid of the subject is substantially undetectable 30 days after implantation. In some embodiments, the concentration of the recombinant native human IL-2 in the blood of the subject is substantially undetectable 1 day after implantation and is at least 3000 μg/ml, 4000 μg/ml, 5000 μg/ml, 10000 μg/ml, 15000 μg/ml, or 20000 μg/ml in the pleural fluid of the subject.

In some embodiments, the subject is administered (e.g., implanted, or delivered) about 0.01 μg/kg/day to about 20 μg/kg/day, about 0.1 μg/kg/day to about 20 μg/kg/day, about 1 μg/kg/day to about 20 μg/kg/day, about 2 μg/kg/day to about 20 μg/kg/day, about 5 μg/kg/day to about 20 μg/kg/day, about 7.5 to about 20 μg/kg/day, about 9 μg/kg/day to about 20 μg/kg/day, about 10 μg/kg/day to about 20 μg/kg/day, about 11 μg/kg/day to about 20 μg/kg/day, about 12 μg/kg/day to about 20 μg/kg/day, about 13 μg/kg/day to about 20 μg/kg/day, about 14 μg/kg/day to about 15 μg/kg/day, about 15 μg/kg/day to about 20 μg/kg/day, about 10 μg/kg/day to about 15 μg/kg/day, about 11 μg/kg/day to about 15 μg/kg/day, about 12 μg/kg/day to about 15 μg/kg/day, about 13 μg/kg/day to about 15 μg/kg/day, about 14 μg/kg/day to about 15 μg/kg/day, about 16 μg/kg/day to about 20 μg/kg/day, about 17 μg/kg/day to about 20 μg/kg/day, about 18 μg/kg/day to about 20 μg/kg/day, about 0.01 μg/kg/day, about 0.05 μg/kg/day, about 0.1 μg/kg/day, about 0.5 μg/kg/day, about 1 μg/kg/day, about 2 μg/kg/day, about 3 μg/kg/day, about 4 μg/kg/day, about 5 μg/kg/day, about 6 μg/kg/day, about 7 μg/kg/day, about 8 μg/kg/day, about 9 μg/kg/day, about 10 μg/kg/day, about 11 μg/kg/day, about 12 μg/kg/day, about 13 μg/kg/day, about 14 μg/kg/day, about 15 μg/kg/day, about 16 μg/kg/day, about 17 μg/kg/day, about 18 μg/kg/day, about 19 μg/kg/day, or about 20 μg/kg/day, of the encapsulated cells as provided herein.

In some embodiments, the implantable construct (encapsulated cells) described herein may be used in a method to modulate (e.g., upregulate) the immune response in a subject. For example, upon administration to a subject, the implantable construct (or an antigenic and/or therapeutic agent disposed within) may modulate (e.g., upregulate) the level of a component of the immune system in a subject (e.g., increasing the level or decreasing the level of an immune system component). Exemplary immune system components that may be modulated by an implantable construct or related method described herein include stem cells (hematopoietic stem cells), NK cells, T cells (e.g., an adaptive T cell (e.g., a helper T cell, a cytotoxic T cell, memory T cell, or regulatory T cell) or an innate-like T cell (e.g., natural killer T cell, mucosal-associated invariant T cell, or gamma delta T cell), B cells, an antibody or fragment thereof, or other another component. In an embodiment, the modulation comprises increasing or decreasing the activation of a T cell or other immune system component (e.g., by about 5%, 10%, 15%, 20%, 25%, 30%, 35%, 40%, 45%, 50%, 55%, 60%, 65%, 70%, 75%, 80%, 85%, 90%, 95%, 99%, or more compared with a control). In some embodiments, the encapsulated cells (implantable construct) can be used to activate CD4 positive and/or CD8 positive immune cells.

The implantable construct described herein may be used to modulate the immune response in a subject for a specific period of time. For example, administration of the implantable construct (or an antigenic and/or therapeutic agent disposed within) may activate the immune response (e.g., by increase in the level of an immune system component) in a subject for at least 1 hour, 2 hours, 3 hours, 4 hours, 6 hours, 8 hours, 10 hours, 12 hours, 16 hours, 20 hours, 1 day, 1.5 days, 2 days, 3 days, 4 days, 5 days, 6 days, 1 week, 1.5 weeks, 2 weeks, 3 weeks, 4 weeks, 5 weeks, 6 weeks, 2 months, 2.5 months, 3 months, 4 months, 5 months, 6 months, or longer. In an embodiment, administration of the implantable construct activates the immune response (e.g., by increase in the level of an immune system component) in a subject between 1 hour and 1 month, 1 hour and 3 weeks, 1 hour and 2 weeks, 1 hour and 1 week, 6 hours and 1 week, or 6 hours and 3 days. In an embodiment, implantation of the implantable construct (e.g., an implantable construct described herein) results in upregulation of T cells in a subject, e.g., as measured by a blood test, for at least 1 day.

The implantable constructs described herein may further comprise an additional pharmaceutical agent, such as an anti-proliferative agent, anti-cancer agent, anti-inflammatory agent, an immunomodulatory agent, or a pain-relieving agent, e.g., for use in combination therapy. The additional pharmaceutical agent may be disposed in or on the implantable construct or may be produced by a cell disposed in or on the implantable construct. In an embodiment, the additional pharmaceutical agent is small molecule, a protein, a peptide, a nucleic acid, an oligosaccharide, or other agent.

In an embodiment, the additional pharmaceutical agent is an anti-cancer agent. In some embodiments, the anti-cancer agent is a small molecule, a kinase inhibitor, an alkylating agent, a vascular disrupting agent, a microtubule targeting agent, a mitotic inhibitor, a topoisomerase inhibitor, an anti-angiogenic agent, or an anti-metabolite. In an embodiment, the anti-cancer agent is a taxane (e.g., paclitaxel, docetaxel, larotaxel or cabazitaxel). In an embodiment, the anti-cancer agent is an anthracycline (e.g., doxorubicin). In some embodiments, the anti-cancer agent is a platinum-based agent (e.g., cisplatin or oxaliplatin). In some embodiments, the anti-cancer agent is a pyrimidine analog (e.g., gemcitabine). In some embodiments, the anti-cancer agent is chosen from camptothecin, irinotecan, rapamycin, FK506, 5-FU, leucovorin, or a combination thereof. In other embodiments, the anti-cancer agent is a protein biologic (e.g., an antibody molecule), or a nucleic acid therapy (e.g., an antisense or inhibitory double stranded RNA molecule).

In an embodiment, the additional pharmaceutical agent is an immunomodulatory agent, e.g., one or more of an activator of a costimulatory molecule, an inhibitor of an immune checkpoint molecule, or an anti-inflammatory agent. In an embodiment, the immunomodulatory agent is an inhibitor of an immune checkpoint molecule (e.g., an inhibitor of PD-1, PD-L1, LAG-3, TIM-3 or CTLA4, or any combination thereof). In some embodiments, the immunomodulatory agent is a cancer vaccine.

In some embodiments, the immunomodulatory agent is an inhibitor of PD-1, PD-L1, PD-L2, CTLA4, TIM3, LAG3, VISTA, BTLA, TIGIT, LAIR1, CD73, CD160, 2B4 and/or TGFR beta. In one embodiment, the inhibitor of an immune checkpoint molecule inhibits PD-1, PD-L1, LAG-3, TIM-3 or CTLA4, or any combination thereof. Inhibition of an inhibitory molecule can be performed at the DNA, RNA or protein level. In some embodiments, an inhibitory nucleic acid (e.g., a dsRNA, siRNA or shRNA), can be used to inhibit expression of an inhibitory molecule. In other embodiments, the inhibitor of an inhibitory signal is, a polypeptide e.g., a soluble ligand (e.g., PD-1-Ig or CTLA-4 Ig), or an antibody or antigen-binding fragment thereof, that binds to the inhibitory molecule; e.g., an antibody or fragment thereof that binds to PD-1, PD-L1, PD-L2, CTLA4, TIM3, LAG3, VISTA, BTLA, TIGIT, LAIR1, CD73, CD160, 2B4 and/or TGFR beta, or a combination thereof. In some embodiments, the immunomodulatory agent is an anti-inflammatory agent, e.g., an anti-inflammatory agent as described herein. In an embodiment, the anti-inflammatory agent is an agent that blocks, inhibits, or reduces inflammation or signaling from an inflammatory signaling pathway. In an embodiment, the anti-inflammatory agent inhibits or reduces the activity of one or more of any of the following an immune component of the subject. In an embodiment, the anti-inflammatory agent is an IL-1 or IL-1 receptor antagonist, such as anakinra, rilonacept, or canakinumab. In an embodiment, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In an embodiment, the anti-inflammatory agent is a TNF-α antagonist, e.g., an anti-TNF-α antibody, such as infliximab (REMICADE®), golimumab (SIMPONI®), adalimumab (HUMIRA®), certolizumab pegol (CIMZIA®) or etanercept. In one embodiment, the anti-inflammatory agent is a corticosteroid, e.g., as described herein.

In some embodiments, a method of providing systemic treatment to a subject with cancer, the method comprising implanting in the cavity of the subject a pharmaceutical composition comprising a population of encapsulated cells comprising a heterologous oligonucleotide molecule encoding the native human IL-2; and administering an immunomodulatory agent; whereby the pharmaceutical composition stimulates the activation of immune cells in the cavity and the activated immune cells migrate to a region of the subject that is distal to the cavity to treat the cancer systemically in the subject, is provided.

In some embodiments, a method of providing systemic treatment to a subject with cancer, the method comprising implanting in the cavity of the subject a pharmaceutical composition comprising a population of encapsulated cells comprising a heterologous oligonucleotide molecule encoding the native human cytokine; and administering an immunomodulatory agent; whereby the pharmaceutical composition activates immune cells and the activated immune cells migrate out of the cavity to treat the cancer in the subject, is provided. In some embodiments, the native human cytokine is IL-1, IL-1α, IL-1β, IL-1RA, IL-4, IL-5, IL-6, IL-7, IL-8, IL-9, IL-10, IL-11, IL-12, IL-12a, IL-12b, IL-13, IL-14, IL-15, IL-16, IL-17, G-CSF, GM-CSF, IL-20, IL-23, IFN-α, IFN-β, IFN-γ, CD154, LT-β, CD70, CD153, CD178, TRAIL, TNF-α, TNF-β, SCF, M-CSF, MSP, 4-1BBL, LIF, OSM, or any combination thereof. In some embodiments, the native human cytokine is IL-2. In some embodiments, a method of providing systemic treatment to a subject with cancer, the method comprising implanting in the cavity of the subject a pharmaceutical composition comprising a population of encapsulated cells comprising a heterologous oligonucleotide molecule encoding the native human IL-2; and administering an immunomodulatory agent; whereby the pharmaceutical composition activates immune cells and the activated immune cells migrate out of the cavity to treat the cancer in the subject, is provided.

In some embodiments, the additional therapeutic is an immunomodulatory agent. In some embodiments, the immunomodulatory agent is an inhibitor of PD-1, PD-L1, PD-L2, CTLA4, TIM3, LAG3, VISTA, BTLA, TIGIT, LAIR1, CD73, CD160, 2B4 and/or TGFRβ. In some embodiments, the inhibitor is an anti-PD-1 antibody, anti-PD-L1 antibody, anti-PD-L2 antibody, anti-CTLA4 antibody, anti-TIM3 antibody, anti-LAG3 antibody, anti-VISTA antibody, anti-BTLA antibody, anti-TIGIT antibody, anti-LAIR1 antibody, anti-CD73 antibody, anti-CD160 antibody, anti-2B4 antibody, anti-TGFRβ antibody, or any combination thereof. In some embodiments, the anti-PD-1 antibody is selected from pembrolizumab, nivolumab, cemiplimab, atezolizumab, dostralimab, durvalumab, avelumab, or any combination thereof. In some embodiments, the anti-PD-1 antibody is pembrolizumab. In some embodiments, the anti-PD-1 antibody is nivolumab. In some embodiments, the anti-PD-1 antibody is cemiplimab. In some embodiments, the anti-PD-1 antibody is atezolizumab. In some embodiments, the anti-PD-1 antibody is dostralimab. In some embodiments, the anti-PD-1 antibody is durvalumab. In some embodiments, the anti-PD-1 antibody is avelumab.

### F. COMPOSITIONS OF IMPLANTABLE CONSTRUCTS

The present disclosure also provides pharmaceutical compositions comprising an implantable construct as provided for herein and optionally a pharmaceutically acceptable excipient. In some embodiments, the implantable construct is provided in an effective amount in the pharmaceutical composition. In some embodiments, the effective amount is a therapeutically effective amount. In some embodiments, the effective amount is a prophylactically effective amount. In some embodiments, the effective amount is an amount that produces an effective amount of native human IL-2.

The present disclosure also provides pharmaceutical compositions comprising an implantable construct and an additional therapeutic as provided for herein and optionally a pharmaceutically acceptable excipient. In some embodiments, the implantable construct is provided in an effective amount in the pharmaceutical composition. In some embodiments, the effective amount is a therapeutically effective amount. In some embodiments, the effective amount is a prophylactically effective amount. In some embodiments, the effective amount is an amount that produces an effective amount of native human cytokine. In some embodiments, the native human cytokine is IL-1, IL-1α, IL-1β, IL-1RA, IL-4, IL-5, IL-6, IL-7, IL-8, IL-9, IL-10, IL-11, IL-12, IL-12a, IL-12b, IL-13, IL-14, IL-15, IL-16, IL-17, G-CSF, GM-CSF, IL-20, IL-23, IFN-α, IFN-β, IFN-γ, CD154, LT-β, CD70, CD153, CD178, TRAIL, TNF-α, TNF-β, SCF, M-CSF, MSP, 4-1BBL, LIF, OSM, or any combination thereof. In some embodiments, the native human cytokine is IL-2.

Pharmaceutical compositions described herein can be prepared by any method known in the art of pharmacology. In general, such preparatory methods include the steps of bringing the implantable construct into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the implantable construct may be generally equal to the dosage of the antigenic and/or therapeutic agent which would be administered to a subject and/or a convenient fraction of such a dosage such as, for example, one-half or one-third of such a dosage.

In some embodiments, the pharmaceutical compositions are frozen or cryopreserved. In some embodiments, the pharmaceutical compositions are not frozen or not cryopreserved.

Relative amounts of the implantable construct, the pharmaceutically acceptable excipient, and/or any additional ingredients in a pharmaceutical composition of the disclosure will vary, depending upon the identity, size, and/or condition of the subject treated and further depending upon the route by which the composition is to be administered. By way of example, the composition may comprise between 0.1% and 100% (w/w) of any component.

For ophthalmic use, provided compounds, compositions, and devices may be formulated as micronized suspensions or in an ointment such as petrolatum.

In an embodiment, the release of an antigenic, therapeutic, or additional pharmaceutical agent is released in a sustained fashion. In order to prolong the effect of a particular agent, it is often desirable to slow the absorption of the agent from injection. This can be accomplished by the use of a liquid suspension of crystalline or amorphous material with poor water solubility. The rate of absorption of the agent then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

The implantable constructs provided herein are typically formulated in dosage unit form, e.g., single unit dosage form, for ease of administration and uniformity of dosage. It will be understood, however, that the total daily usage of the compositions of the present disclosure will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific therapeutic agent employed; and like factors well known in the medical arts.

Also provided for herein, are a population of encapsulated cells prepared according to a method as provided for herein.

In some embodiments, a suspension of encapsulated cells is provided. In some embodiments, the suspension comprises a population of encapsulated cells as provided for herein. In some embodiments, the encapsulated cells are encapsulated by a polymeric hydrogel, and the suspension comprises a crosslinking solution that comprises a sugar alcohol, a buffer, a metal salt, and a surfactant. In some embodiments, the cells are ARPE-19 cells. In some embodiments, the surfactant is TWEEN 20 (polysorbate 20). In some embodiments, the buffer is HEPES buffer. In some embodiments, the sugar alcohol is mannitol. In some embodiments, the metal salt is barium chloride.

In some embodiments, suspensions of encapsulated cells are provided, wherein the suspension comprises a population of encapsulated cells as provided for herein, wherein the encapsulated cells are encapsulated by a polymeric hydrogel, and a storage buffer, such as DMEM/F12 cell culture media. In some embodiments, the suspended encapsulated cells retain viability for at least 10, 20, or 30 days.

In some embodiments, the suspension provided for herein are substantially free of plasmalyte buffer.

### G. METHODS OF ADMINISTRATION

The implantable construct and a pharmaceutical composition thereof may be administered or implanted orally, parenterally (including subcutaneous, intramuscular, intravenous and intradermal), intrapleurally, by inhalation spray, topically, rectally, nasally, buccally, vaginally, via an implanted reservoir, or via a device or method provided herein. In some embodiments, provided compounds or compositions are administrable intravenously and/or orally. In some embodiments, provided compounds or compositions are administered into the pleural space. In an embodiment, the implantable construct is injected subcutaneously. In an embodiment, the implantable construct is injected into the pleural cavity. In an embodiment, the implantable construct is injected into the pleural cavity. In an embodiment, the implantable constructed is delivered (e.g., injected into the peritoneal or pleural cavity) to the subject using a device, e.g., a cannula, a catheter, or as provided herein. In some embodiments, the implantable constructs and pharmaceutical compositions thereof, may be administered or implanted in or on a certain region of the body, such as the pleural cavity. Exemplary sites of administration or implantation include the peritoneal cavity (e.g., lesser sac), adipose tissue, heart, eye, muscle, spleen, lymph node, esophagus, nose, sinus, teeth, gums, tongue, mouth, throat, small intestine, large intestine, thyroid, bone (e.g. hip or a joint), breast, cartilage, vagina, uterus, fallopian tube, ovary, penis, testicles, blood vessel, liver, kidney, central nervous system (e.g., brain, spinal cord, nerve), ear (e.g., cochlea), or pleural cavity.

The term “parenteral” as used herein includes subcutaneous, intravenous, intramuscular, intraocular, intravitreal, intra-articular, intra-synovial, intrasternal, intrathecal, intrahepatic, intraperitoneal, intralesional, and intracranial injection, or infusion techniques.

Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

The therapeutic agent administered may be at dosage levels sufficient to deliver from about 0.00001 mg/kg to about 100 mg/kg, from about 0.0001 mg/kg to about 100 mg/kg, from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 0.001 mg/kg to about 1 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

In some embodiments, methods of delivering a native IL-2 to the pleural cavity of subject, the method comprising implanting, or delivering to, the pleural cavity a pharmaceutical composition comprising a population of encapsulated cells comprising a heterologous oligonucleotide molecule encoding the native human IL-2 are provided. In some embodiments, the implanting, or delivering, is achieved using methods and/or devices as provided herein.

In some embodiments, methods of delivering a native cytokine and an additional therapeutic, such as those provided herein, to the subject, the method comprising implanting, or delivering to, the subject a pharmaceutical composition comprising a population of encapsulated cells comprising a heterologous oligonucleotide molecule encoding the native human cytokine and an additional therapeutic, such as those provided herein, are provided. In some embodiments, the native human cytokine is IL-1, IL-1α, IL-1β, IL-1RA, IL-4, IL-5, IL-6, IL-7, IL-8, IL-9, IL-10, IL-11, IL-12, IL-12a, IL-12b, IL-13, IL-14, IL-15, IL-16, IL-17, G-CSF, GM-CSF, IL-20, IL-23, IFN-α, IFN-β, IFN-γ, CD154, LT-β, CD70, CD153, CD178, TRAIL, TNF-α, TNF-β, SCF, M-CSF, MSP, 4-1BBL, LIF, OSM, or any combination thereof. In some embodiments, the native human cytokine is IL-2. In some embodiments, methods of delivering a native IL-2 and an additional therapeutic, such as those provided herein, to the subject, the method comprising implanting, or delivering to, the subject a pharmaceutical composition comprising a population of encapsulated cells comprising a heterologous oligonucleotide molecule encoding the native human IL-2 and an additional therapeutic, such as those provided herein, are provided. In some embodiments, the implanting, or delivering, is achieved using methods and/or devices as provided herein.

The constructs (e.g., encapsulated cells) can be prepared according to any known method. For example, in some embodiments, methods of preparing encapsulated cells producing a recombinant protein are provided. In some embodiments, the methods comprise feeding through a coaxial needle a first composition comprising a polymeric hydrogel and a second composition comprising cells to be encapsulated suspended in a polymeric hydrogel to drop into a crosslinking solution to form the encapsulated cells, wherein the crosslinking solution comprises a sugar alcohol, a buffer, a metal salt, and a surfactant. In some embodiments, the cells to be encapsulated comprise an oligonucleotide molecule encoding native human IL-2. In some embodiments, the oligonucleotide encoding native human IL-2 comprises a sequence of SEQ ID NO: 1. In some embodiments, the cell produces recombinant native human IL-2 protein. In some embodiments, the IL-2 protein is formed from an amino acid sequence of SEQ ID NO: 2.

The cells can be any type of cell. In some embodiments, the cell is a mammalian cell. In some embodiments, the cell is an epithelial cell. In some embodiments, the cell is a RPE cell. In some embodiments, the cell is a ARPE-19 cell, ARPE-19-SEAP-2-neo cell, RPE-J cell, and hTERT RPE-1 cell. In some embodiments, the cell is an engineered RPE cell. In some embodiments, the engineered cell is derived from the ARPE-19 cell line. In some embodiments, the cell is as provided herein. In some embodiments, the surfactant is TWEEN 20 (polysorbate 20). In some embodiments, the buffer is HEPES buffer. In some embodiments, the sugar alcohol is mannitol. In some embodiments, the metal salt is barium chloride.

In some embodiments, the method comprises washing the encapsulated cells produced according to the methods provided for herein in a buffer solution produced. In some embodiments, the washing step removes substantially all or all of the free barium or barium chloride.

In some embodiments, the encapsulated cells prepared according to the methods provided herein are stored in a storage buffer, such as DMEM/F12 cell culture media. In some embodiments, the stored cells retain viability for at least 10, 20, or 30 days. In some embodiments, the storage buffer is substantially free of plasmalyte buffer.

In some embodiments, the implantable construct, or a pharmaceutical composition thereof is entirely or partially disposed within an implantable element. The implantable element may comprise an enclosing element that encapsulates or coats a cell, in part or in whole. In some embodiments, an implantable element comprises an enclosing component that is formed, or could be formed, in situ on or surrounding a cell, e.g., a plurality of cells, e.g., a cluster of cells, or on a microcarrier, e.g., a bead, or a matrix comprising a cell or cells (referred to herein as an “in-situ encapsulated implantable element”). In some embodiments, an implantable element comprises an enclosing component that is preformed prior to combination with the enclosed cell, e.g., a plurality of cells, e.g., a cluster of cells, or a microcarrier, e.g., a bead or a matrix comprising a cell (referred to herein as device-based-implantable element, or DB-implantable element).

The implantable elements described herein include devices or materials, for example, devices or materials associated with the implantable constructs and pharmaceutical compositions thereof described herein. In some embodiments, a device or material may be associated with the implantable construct and pharmaceutical composition thereof as provided herein. In some embodiments, the implantable element is administered into the pleural space. In some embodiments, a device or material associated with the implantable construct and pharmaceutical composition thereof as provided herein is administered into the pleural space.

In some embodiments, devices included herein include devices that are configured with a lumen, e.g., a lumen having one, two or more openings, e.g., tubular devices. A typical stent is an example of a device configured with a lumen and having two openings. Other examples include shunts, or needles. In some embodiments, devices included herein include flexible devices, e.g., devices that are configured to conform to the shape of the body. In some embodiments, devices included herein include devices comprising an element that stabilizes the location of the device, e.g., an adhesive, or fastener, e.g., a torque-based or friction based fastener, e.g., a screw or a pin. In some embodiments, devices included herein include devices configured to release a substance, e.g., an therapeutic agent, e.g., an encapsulated cell, e.g., an implantable construct. In some embodiments, the implantable construct is as provided herein or a pharmaceutical composition thereof. In some embodiments, the therapeutic agent is a cell, cell product, tissue, tissue product, protein, hormone, enzyme, antibody, antibody fragment, antigen, epitope, drug, vaccine, or any derivative thereof. In some embodiments, the device is an AccuStick™.

In some embodiments, devices provided herein include articulable devices that are configured to change conformation in response to a signal or movement of the body, e.g., an artificial joint, e.g., a knee, hip, or other artificial joint. Exemplary devices are provided herein.

In some embodiments, devices included herein include stents or other devices that are configured to be placed partially or entirely in a lumen of the body. A vascular stent is a stent configured for disposition entirely, or partially, within a lumen of the vasculature, e.g., a coronary, urinary, biliary, venous, or coronary stent. Stents can be configured to have other properties, e.g., to be expandable, or to release or elute a substance, e.g., an implantable construct. Stents can be configured so as to affect the shape of adjacent tissue, e.g., to keep a passage open. Typically a stent can be made of metal, plastic, or a material described herein. Stents can be configured for use in coronary heart disease, carotid artery disease, high blood pressure, peripheral arterial disease, aneurysm, stroke, atherosclerosis, an aged subject (e.g., at least 60 years of age), or a subject undergoing coronary angioplasty. Exemplary stents include: coronary, aortic, drug-eluting, intracranial, pancreatic, carotid, iliac, renal, femoral, ureteral, bladder fetal, duodenal, biliary shunts. Shunts can comprise stainless steel, gold, titanium, cobalt-chromium alloy, tantalum alloy, nitinol, silicone, polyurethane, polyesters, polyorthoesters, polyanhydrides, or collagen.

In some embodiments, devices included herein include shunts or other devices that are configured to connect to connect, and typically provide fluid connection with, a first part of the body, e.g., a first organ, and a second part of the body, or the exterior. A shunt can be configured to be permanent or temporary. Typically a shunt can be made of metal, plastic, or a material described herein. Shunts can be configured to have other properties, e.g., to be expandable, or to release or elute a substance, e.g., an implantable construct. Shunts can be configured for use in the eye, e.g., a glaucoma shunt, the CNS, e.g., the brain or spinal column, a cavity, e.g., the peritoneal cavity, or an organ. Shunts can be configured for use in the treatment of coronary heart disease, carotid artery disease, high blood pressure, peripheral arterial disease, aneurysm, stroke, atherosclerosis, or to treat a subject (e.g., at least 60 years of age), or a subject undergoing coronary angioplasty. Exemplary shunts include: peritoneal, endolymphatic, intracranial, and tympanostomy shunts.

In some embodiments, devices included herein include scaffoldings (also termed “scaffolds”) that are configured to allow invasion of the device by tissue of the body. Scaffoldings can be configured as meshes, networks, or as porous. Typically a scaffolding will comprise an element or elements that provide dimensional stability. Scaffoldings can be configured to be permanent or temporary. Typically a scaffolding can be made of metal, plastic, or a material described herein. Scaffoldings can be configured to have any of a variety of properties, e.g., to promote growth, or growth or regeneration is a desired direction, or to release or elute a substance, e.g., an implantable construct. Scaffoldings can be configured of flexible material or nonflexible material. Scaffoldings include bone scaffoldings, for the promotion of growth of bone or surrounding tissues, e.g., configured for use in breaks, fractures, osteoporosis, or joint replacement.

In some embodiments, devices included herein include ocular devices that are configured for placement on the eye, in the eye, or in or on the tissues surrounding the eye. Such devices include eye mountable devices, e.g., contact lenses. Such devices also include intraocular devices, including intraocular lenses, e.g., phasic intraocular lenses, implantable lens (e.g., made of polymers), e.g., for cataract treatment/surgery, shunts, e.g., glaucoma shunts, or devices for the release of a substance, e.g., an implantable construct. Typically an ocular device can be made of metal, plastic, or a material described herein. Ocular devices can be configured to release or elute a substance, e.g., an implantable construct.

In some embodiments, devices included herein include soft tissue prosthetic devices. Soft tissue prosthetic devices can be configured to have any of a variety of properties, e.g., to promote growth, or to release or elute a substance, e.g., an implantable construct.

In some embodiments, devices included herein include catheters, e.g., balloon catheters, configured to promote opening of a lumen, typically a vascular lumen, e.g., a coronary vascular lumen. Catheters can be configured to be permanent or temporary. Typically a catheter can be made of metal, plastic, or a material described herein. Catheters can be configured to have any of a variety of properties, e.g., to promote healing, to be expandable, or to release or elute a substance, e.g., an implantable construct. Exemplary catheters include: hemodialysis, biliary, peritoneal, subclavian, suprapubic, ventricular, atrial, intravascular, subcutaneous catheters. They can comprise silicone rubber, nylon, polyurethane, polyethylene terephthalate (PET), latex, thermoplastic elastomers. Some catheters have a thin hydrophilic surface coating.

In some embodiments, devices included herein include ports or other devices that are configured to provide access to the body. A port can be configured to allow continuous, or intermittent connection to a reservoir containing a substance, e.g, an implantable construct. Ports can be configured to have other properties, e.g., to be closeable, or to release or elute a substance, e.g., a therapeutic agent. Ports can be configured so as to conform to the surface of the body. Typically a port can be made of metal, plastic, or a material described herein. Ports can be configured for use subjects having chronic illness or cancer.

In some embodiments, devices included herein include extracorporeal devices, e.g., devices through which a tissue or fluid, e.g., blood or spinal fluid, is passed, including, e.g., renal dialysis device, port, and tubing, e.g., dialysis tubing. Extracorporeal devices can be configured to have other properties, e.g., to be closeable, or to release or elute a substance, e.g., an implantable construct.

In some embodiments, devices included herein include, orthopedic fixation devices, dental implants, skin covering devices; dialysis media, and drug-delivery devices, and artificial or engineered organs, e.g., hearts. Other devices included herein include: silicon implants, drainage devices, e.g., bladder drainage devices, cell selection systems, adhesives, e.g., cement, clamp, clip, contraceptive devices, intrauterine devices, corneal implants, dermal implants, dental implants, ocular implants, intragastric implants, facial implants, penile implants, implants for control of incontinence, e.g., urine or fecal, defibrillators, dosimeters, electrodes, pumps, e.g., infusion pumps, filters, embolization devices, fastener, fillers, fixatives, grafts, hearing aids, cardio or heart-related devices, e.g., pacemakers and valves, batteries or power sources, hemostatic agents, incontinence devices, intervertebral body fusion devices, intraoral devices, lenses, meshes, needles, nervous system stimulators, patches, peritoneal access devices, plates, plugs, pressure monitoring devices, rings, transponders, hip implants, bone implants, or valves. Also included are devices used in one or more of: anesthesiology, cardiovascular, clinical chemistry, dental, ear, nose, throat, gastroenterology, urology, general hospital, hematology, immunology, microbiology, neurology, obstetrics/gynecology, ophthalmic, orthopedic, pathology, physical medicine, radiology, general or plastic surgery, and/or clinical toxicology. In some embodiments, devices include clips, e.g., anchor fascial, aneurysm, hemostatic, coronary artery bypass, ophthalmic tantalum, tubal occlusion, vascular, and marker radiographic clips. Clips can comprise titanium, titanium-aluminum alloy, or cobalt-chromium-nickel-molybdenum-iron alloy.

In some embodiments, devices include meshes, e.g., absorbable/non-absorbable, collagen, synthetic/non-synthetic meshes. Meshes can comprise: polyglycolic acid, polypropylene, polyethylene terephthalate, nonocryl (poliglecaprone 25), cellulose, macroporous polyester, poly-4-hydroxybutrate, polytetrafluoroethylene, biologics (human dermis, porcine dermis, porcine small intestine submucosa, bovine pericardium), or fibroin.

In some embodiments, devices include plugs, e.g., a plug, e.g., a biopsy plug, e.g., a lung biopsy plug, made e.g., of polyethylene glycol (PEG) hydrogel. Other plugs are configured for cerebrospinal fluid leakage (Dural), arteries (BioGlue), lung tissue (AeriSeal). Exemplary materials include polyethylene glycol ester and trilysine amine (Dural), bovine serum albumin and glutaraldehyde (BioGlue), or aminated polyvinyl alcohol and glutaraldehyde (AeriSeal).

In some embodiments, devices included herein include FDA class 1, 2, or 3 devices, e.g., devices that are unclassified or not classified, or classified as a humanitarian use device (HUD).

In some embodiments, the implantable construct and pharmaceutical composition thereof is administered using a device as provided herein. In some embodiments, the implantable construct and pharmaceutical composition thereof is administered intrapleurally using a device as provided herein.

Exemplary components or materials can be purely structural, therapeutic, or both. A device can comprise a biomolecule component, e.g., a carbohydrate, e.g., a polysaccharide, e.g., a marine polysaccharide, e.g., alginate, agar, agarose, carrageenans, cellulose and amylose, chitin and chitosan; cross-linked polysaccharides, e.g., cross-linked by diacrylates; or a polysaccharide or derivative/modification thereof described in, e.g., Laurienzo (2010), Mar. Drugs. 8.9:2435-65.

In some embodiments, the following embodiments are provided:
1. A method of treating a mesothelioma, in a subject, the method comprising implanting, or delivering to, the pleural cavity a pharmaceutical composition comprising a population of encapsulated cells comprising a heterologous oligonucleotide molecule encoding the native human IL-2.
2. The method of embodiment 1, wherein the mesothelioma is a pleural mesothelioma, a malignant pleural mesothelioma, or a diffuse pleural mesothelioma.
3. A method of treating a mesothelioma, in a subject by generating memory immunity, the method comprising implanting, or delivering to, the pleural cavity a pharmaceutical composition comprising a population of encapsulated cells comprising a heterologous oligonucleotide molecule encoding the native human IL-2.
4. The method of embodiment 3, wherein the mesothelioma is a pleural mesothelioma, a malignant pleural mesothelioma, or a diffuse pleural mesothelioma.
5. A method of delivering a native IL-2 to the pleural cavity of subject, the method comprising implanting, or delivering to, the pleural cavity a pharmaceutical composition comprising a population of encapsulated cells comprising a heterologous oligonucleotide molecule encoding the native human IL-2.
6. A method of treating a disease, in a subject, the method comprising implanting, or delivering to, the pleural cavity of the subject a pharmaceutical composition comprising a population of encapsulated cells comprising a heterologous oligonucleotide molecule encoding the native human IL-2.
7. The method of embodiment 2, wherein the disease is as provided herein.
8. A method of treating a pleural disease or condition, in a subject, the method comprising implanting, or delivering to, the pleural cavity a pharmaceutical composition comprising a population of encapsulated cells comprising a heterologous oligonucleotide molecule encoding the native human IL-2.
9. A method of treating a pleural disease or condition, in a subject by generating memory immunity, the method comprising implanting, or delivering to, the pleural cavity a pharmaceutical composition comprising a population of encapsulated cells comprising a heterologous oligonucleotide molecule encoding the native human IL-2.
10. The method of embodiments 8 or 9, wherein the pleural disease or condition is pleural cancer, pleural metastatic disease, pleurisy, lung infection, viral pneumonia, bacterial pneumonia, idiopathic pulmonary fibrosis, acute respiratory distress syndrome, pleural thickening, pleural pseudotumor, pleural plaque, extrapleural hematoma, Castleman disease, hemangioendothelioma, splenosis, paramalignang effusion, pleural effusion, pneumothorax, hemothorax, reactive pleuritis.
11. The method of embodiment 10, wherein the pleural cancer is mesothelioma lung cancer, metastases, malignant mesothelioma, lymphoma, malignant fibrous tumor, sarcoma, askin tumor, extraskeletal osteosarcoma, malignant fibrous histiocytoma, solitary fibrous tumor, lipoma, mesothelial cyst, calcifying fibrous pseudotumor, primary effusion lymphoma.
12. A method of providing systemic treatment to a subject with cancer, the method comprising implanting in the pleural cavity of the subject a pharmaceutical composition comprising a population of encapsulated cells comprising a heterologous oligonucleotide molecule encoding the native human IL-2, whereby the pharmaceutical composition stimulates the activation of immune cells in the pleural cavity and the activated immune cells migrate to a region of the subject that is distal to the pleural cavity to treat the cancer systemically in the subject.
13. A method of providing systemic treatment to a subject with cancer, the method comprising implanting in the pleural cavity of the subject a pharmaceutical composition comprising a population of encapsulated cells comprising a heterologous oligonucleotide molecule encoding the native human IL-2, whereby the pharmaceutical composition activates immune cells and the activated immune cells migrate out of the pleural cavity to treat the cancer in the subject.
14. The method of embodiments 12 or 13, wherein the subject has fewer side effects as compared to a subject that is administered the pharmaceutical composition systemically, such as intravenously.
15. The method of any one of embodiments 12-14, wherein the activated immune cells are CD8 positive effector T cells.
16. The method of any one of embodiments 12-15, wherein the effector T cells are selectively activated and expanded at least 1, 2, 3, 4, or 5 times as compared to Tregs in the pleural cavity.
17. The method of any one of embodiments 12-15, wherein the effector T cells are selectively activated and expanded at least 1, 2, 3, 4, or 5 times as compared to Tregs systemically.
18. The method of any one of embodiments 1-17, wherein the subject is administered about 0.01 μg/kg/day to about 20 μg/kg/day, about 0.1 μg/kg/day to about 20 μg/kg/day, about 1 μg/kg/day to about 20 μg/kg/day, about 2 μg/kg/day to about 20 μg/kg/day, about 5 μg/kg/day to about 20 μg/kg/day, about 7.5 to about 20 μg/kg/day, about 9 μg/kg/day to about 20 μg/kg/day, about 10 μg/kg/day to about 20 μg/kg/day, about 11 μg/kg/day to about 20 μg/kg/day, about 12 μg/kg/day to about 20 μg/kg/day, about 13 μg/kg/day to about 20 μg/kg/day, about 14 μg/kg/day to about 15 μg/kg/day, about 15 μg/kg/day to about 20 μg/kg/day, about 10 μg/kg/day to about 15 μg/kg/day, about 11 μg/kg/day to about 15 μg/kg/day, about 12 μg/kg/day to about 15 μg/kg/day, about 13 μg/kg/day to about 15 μg/kg/day, about 14 μg/kg/day to about 15 μg/kg/day, about 16 μg/kg/day to about 20 μg/kg/day, about 17 μg/kg/day to about 20 μg/kg/day, about 18 μg/kg/day to about 20 μg/kg/day, about 0.01 μg/kg/day, about 0.1 μg/kg/day, about 1 μg/kg/day, about 2 μg/kg/day, about 3 μg/kg/day, about 4 μg/kg/day, about 5 μg/kg/day, about 6 μg/kg/day, about 7 μg/kg/day, about 8 μg/kg/day, about 9 μg/kg/day, about 10 μg/kg/day, about 11 μg/kg/day, about 12 μg/kg/day, about 13 μg/kg/day, about 14 μg/kg/day, about 15 μg/kg/day, about 6 μg/kg/day, about 17 μg/kg/day, about 18 μg/kg/day, about 19 μg/kg/day, or about 20 μg/kg/day, of the encapsulated cells.
19. The method of any one of embodiments 1-18, wherein the concentration of native human IL-2 in the pleural fluid at day 1 post implantation is at least 3000 μg/ml, 4000 μg/ml, 5000 μg/ml, 10000 μg/ml, 15000 μg/ml, or 20000 μg/ml.
20. The method of any one of embodiments 1-19, wherein the concentration of the recombinant native human IL-2 in the blood of the subject is substantially undetectable 1 day after implantation.
21. The method of any one of embodiments 1-20, wherein the concentration of the recombinant native human IL-2 in the pleural fluid of the subject is substantially undetectable 30 days after implantation.
22. The method of any one of embodiments 1-21, wherein the concentration of the recombinant native human IL-2 in the blood of the subject is substantially undetectable 1 day after implantation and is at least 3000 μg/ml, 4000 μg/ml, 5000 μg/ml, 10000 μg/ml, 15000 μg/ml, or 20000 μg/ml in the pleural fluid of the subject.
23. The method of any one of embodiments 1-22, wherein the pharmaceutical composition is implanted according to a method or using a device as provided for herein.
24. The method of any one of embodiments 1-23, wherein the oligonucleotide encoding native human IL-2 comprises a sequence of SEQ ID NO: 1:
25. The method of any one of embodiments 1-24, wherein the oligonucleotide encoding native human IL-2 comprises a sequence that is codon-optimized.
26. The population of encapsulated cells of embodiment 25, wherein the codon-optimized oligonucleotide encoding native human IL-2 comprises a sequence having at least 90%, 91%, 92%, 93%, 94%, 95%, 96%, 97%, 98%, 99%, or 100% sequence identity to SEQ ID NO: 3.
27. The method of any one of embodiments 1-26, wherein the cell produces recombinant native human IL-2 protein.
28. The method of any one of embodiments 1-27, wherein the recombinant native human IL-2 protein expressed by the cells comprises the amino acid sequence of: SEQ ID NO: 2.
29. The method of any one of embodiments 1-28, wherein the pharmaceutical composition produces about 1 to about 10, about 1 to about 5, or about 2 to about 4 PCD (picograms/cell/day) of native human IL-2.
30. The method of any one of embodiments 1-29, wherein the encapsulated cells comprises a cell as provided for herein.
31. The method of any one of embodiments 1-30, wherein the encapsulated cells comprise ARPE-19 cells comprising the heterologous oligonucleotide molecule.
32. The method of any one of embodiments 1-31, wherein the encapsulated cells are encapsulated with a polymeric hydrogel.
33. The method of embodiment 32, wherein the polymeric hydrogel comprises chitosan, cellulose, hyaluronic acid, or alginate.
34. The method of embodiments 32 or 33, wherein the polymeric hydrogel comprises alginate.
35. The method of any one of embodiments 32-34, wherein the alginate comprises SLG20.
36. The method of any one of embodiments 1-35, wherein the cells remain viable for at least 15, 20, 25, or 28 days.
37. The method of any one of embodiments 1-36, wherein the encapsulated cells do not proliferate.
38. The method of any one of embodiments 1-37, wherein the encapsulated cells produced a sustained amount of IL-2 for at least 5, 10, 15, 20, or 24 hours.
39. The method of any one of embodiments 1-38, wherein the encapsulated cells can produce a sustained amount of IL-2 for up to 30 days.
40. The method of embodiment 1, further comprising administering an additional therapeutic.
41. The method of embodiment 40, wherein the additional therapeutic is an immunomodulatory agent.
42. The method of embodiment 41, wherein the immunomodulatory agent is an inhibitor of PD-1, PD-L1, PD-L2, CTLA4, TIM3, LAG3, VISTA, BTLA, TIGIT, LAIR1, CD73, CD160, 2B4 and/or TGFRβ.
43. The method of embodiment 42, wherein the inhibitor is an anti-PD-1 antibody, anti-PD-L1 antibody, anti-PD-L2 antibody, anti-CTLA4 antibody, anti-TIM3 antibody, anti-LAG3 antibody, anti-VISTA antibody, anti-BTLA antibody, anti-TIGIT antibody, anti-LAIR1 antibody, anti-CD73 antibody, anti-CD160 antibody, anti-2B4 antibody, anti-TGFRβ antibody, or any combination thereof.
44. A method of delivering a native cytokine and an additional therapeutic to the subject, the method comprising implanting, or delivering to, the subject a pharmaceutical composition comprising a population of encapsulated cells comprising a heterologous oligonucleotide molecule encoding the native human cytokine and further administering a pharmaceutical composition comprising an additional therapeutic.
45. A method of treating a disease or condition, in a subject, the method comprising implanting, or delivering to, the subject a pharmaceutical composition comprising a population of encapsulated cells comprising a heterologous oligonucleotide molecule encoding an IL-2 molecule and further administering a pharmaceutical composition comprising an additional therapeutic.
46. The method of embodiment 44, wherein the disease or condition is a cancer.
47. The method of embodiment 3, wherein the cancer is a mesothelioma.
48. The method of embodiment 47, wherein the mesothelioma is a pleural mesothelioma, peritoneal mesothelioma, pericardial mesothelioma, testicular mesothelioma, epithelioid mesothelioma, sarcomatoid mesothelioma, biphasic mesothelioma, small cell mesothelioma, deciduoid mesothelioma, cystic and papillary mesothelioma, desmoplastic mesothelioma, adenomatoid mesothelioma, heterologous mesothelioma, well-defined papillary cell mesothelioma, or any combination thereof.
49. A method of treating mesothelioma in a subject, the method comprising implanting, or delivering to, the subject a pharmaceutical composition comprising a population of encapsulated cells comprising a heterologous oligonucleotide molecule encoding the native human cytokine and further administering a pharmaceutical composition comprising an additional therapeutic.
50. The method of embodiment 49, wherein the mesothelioma is selected from a pleural mesothelioma, peritoneal mesothelioma, pericardial mesothelioma, testicular mesothelioma, epithelioid mesothelioma, sarcomatoid mesothelioma, biphasic mesothelioma, small cell mesothelioma, deciduoid mesothelioma, cystic and papillary mesothelioma, desmoplastic mesothelioma, adenomatoid mesothelioma, heterologous mesothelioma, well-defined papillary cell mesothelioma, or any combination thereof.
51. The method of any one of embodiments 43-50, wherein the cytokine is IL-1, IL-1α, IL-1β, IL-1RA, IL-2, IL-4, IL-5, IL-6, IL-7, IL-8, IL-9, IL-10, IL-11, IL-12, IL-12a, IL-12b, IL-13, IL-14, IL-15, IL-16, IL-17, G-CSF, GM-CSF, IL-20, IL-23, IFN-α, IFN-β, IFN-γ, CD154, LT-β, CD70, CD153, CD178, TRAIL, TNF-α, TNF-β, SCF, M-CSF, MSP, 4-1BBL, LIF, OSM, or any combination thereof.
52. The method of any one of embodiments 43-51, wherein the additional therapeutic is an immunomodulatory agent.
53. The method of embodiment 52, wherein the immunomodulatory agent is an inhibitor of PD-1, PD-L1, PD-L2, CTLA4, TIM3, LAG3, VISTA, BTLA, TIGIT, LAIR1, CD73, CD160, 2B4 and/or TGFRβ.
54. The method of embodiment 53, wherein the inhibitor is an anti-PD-1 antibody, anti-PD-L1 antibody, anti-PD-L2 antibody, anti-CTLA4 antibody, anti-TIM3 antibody, anti-LAG3 antibody, anti-VISTA antibody, anti-BTLA antibody, anti-TIGIT antibody, anti-LAIR1 antibody, anti-CD73 antibody, anti-CD160 antibody, anti-2B4 antibody, anti-TGFRβ antibody, or any combination thereof.
55. A method of treating mesothelioma in a subject, the method comprising implanting, or delivering to, the subject a pharmaceutical composition comprising a population of encapsulated cells comprising a heterologous oligonucleotide molecule encoding an IL-2 molecule and further administering a pharmaceutical composition comprising an immunomodulatory agent.
56. The method of embodiment 55, wherein the mesothelioma is selected from a pleural mesothelioma, peritoneal mesothelioma, pericardial mesothelioma, testicular mesothelioma, epithelioid mesothelioma, sarcomatoid mesothelioma, biphasic mesothelioma, small cell mesothelioma, deciduoid mesothelioma, cystic and papillary mesothelioma, desmoplastic mesothelioma, adenomatoid mesothelioma, heterologous mesothelioma, well-defined papillary cell mesothelioma, or any combination thereof.
57. The method of embodiments 55 or 56, wherein the immunomodulatory agent is an inhibitor of PD-1, PD-L1, PD-L2, CTLA4, TIM3, LAG3, VISTA, BTLA, TIGIT, LAIR1, CD73, CD160, 2B4 and/or TGFRβ.
58. The method of embodiment 57, wherein the inhibitor is an anti-PD-1 antibody, anti-PD-L1 antibody, anti-PD-L2 antibody, anti-CTLA4 antibody, anti-TIM3 antibody, anti-LAG3 antibody, anti-VISTA antibody, anti-BTLA antibody, anti-TIGIT antibody, anti-LAIR1 antibody, anti-CD73 antibody, anti-CD160 antibody, anti-2B4 antibody, anti-TGFRβ antibody, or any combination thereof.
59. The method of embodiment 58, wherein the anti-PD-1 antibody is selected from pembrolizumab, nivolumab, cemiplimab, atezolizumab, dostralimab, durvalumab, avelumab, or any combination thereof.
60. The method of any one of embodiments 43-59, wherein the treatment results in activation or increase of immune cells.
61. The method of embodiment 60, wherein the activated immune cells are CD4 and CD8 positive T cells.
62. The method of embodiment 60, wherein the increased immune cells are CD4 and CD8 positive effector T cells.
63. The method of any one of embodiments 44-62, wherein the treatment results in macrophage phenotype shift.
64. The method of embodiment 63, wherein the macrophage phenotype shift is from M2-like macrophages to M1-like macrophages.
65. The method of embodiments 63 or 64, wherein the phenotype shift from M2-like macrophages to M1-like macrophages results in reduction of M2-like macrophages and increase in M1-like macrophages.
66. The method of any one of embodiments 63-65, wherein the treatment results in increase in MHC II+ dendritic cells.
67. A method of providing systemic treatment to a subject with cancer, the method comprising
   implanting in a cavity of the subject a pharmaceutical composition comprising a population of encapsulated cells comprising a heterologous oligonucleotide molecule encoding an IL-2 molecule; and
   administering an immunomodulatory agent;
   whereby the pharmaceutical composition stimulates the activation of immune cells in the cavity and the activated immune cells migrate to a region of the subject that is distal to the cavity to treat the cancer systemically in the subject.
68. A method of providing systemic treatment to a subject with cancer, the method comprising
   implanting in a cavity of the subject a pharmaceutical composition comprising a population of encapsulated cells comprising a heterologous oligonucleotide molecule encoding an IL-2 molecule; and
   administering an immunomodulatory agent;
   whereby the pharmaceutical composition activates immune cells and the activated immune cells migrate out of the cavity to treat the cancer in the subject.
69. The method of embodiments 67 or 68, wherein the cancer is a mesothelioma.
70. The method of embodiment 69, wherein the mesothelioma is a pleural mesothelioma, peritoneal mesothelioma, pericardial mesothelioma, testicular mesothelioma, epithelioid mesothelioma, sarcomatoid mesothelioma, biphasic mesothelioma, small cell mesothelioma, deciduoid mesothelioma, cystic and papillary mesothelioma, desmoplastic mesothelioma, adenomatoid mesothelioma, heterologous mesothelioma, well-defined papillary cell mesothelioma, or any combination thereof.
71. The method of any one of embodiments 67-70, wherein the immunomodulatory agent is an inhibitor of PD-1, PD-L1, PD-L2, CTLA4, TIM3, LAG3, VISTA, BTLA, TIGIT, LAIR1, CD73, CD160, 2B4 and/or TGFRβ.
72. The method of embodiment 71, wherein the inhibitor is an anti-PD-1 antibody, anti-PD-L1 antibody, anti-PD-L2 antibody, anti-CTLA4 antibody, anti-TIM3 antibody, anti-LAG3 antibody, anti-VISTA antibody, anti-BTLA antibody, anti-TIGIT antibody, anti-LAIR1 antibody, anti-CD73 antibody, anti-CD160 antibody, anti-2B4 antibody, anti-TGFRβ antibody, or any combination thereof.
73. The method of embodiment 72, wherein the anti-PD-1 antibody is selected from pembrolizumab, nivolumab, cemiplimab, atezolizumab, dostralimab, durvalumab, avelumab, or any combination thereof.
74. The method of any one of embodiments 67-73, wherein the activated immune cells are CD4 and CD8 positive T cells.
75. The method of any one of embodiments 67-31, wherein the cavity is a pleural cavity or the IP space.
76. The method of any one of embodiments 43-75, wherein the additional therapeutic or the immunomodulatory agent is administered 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days following implantation of the pharmaceutical composition comprising a population of encapsulated cells.
77. The method of any one of embodiments 43-76, wherein the additional therapeutic or the immunomodulatory agent is administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days following implantation of the pharmaceutical composition comprising a population of encapsulated cells.
78. The method of any one of embodiments 43-77, wherein the subject is administered about 0.01 μg/kg/day to about 20 μg/kg/day, about 0.1 μg/kg/day to about 20 μg/kg/day, about 1 μg/kg/day to about 20 μg/kg/day, about 2 μg/kg/day to about 20 μg/kg/day, about 5 μg/kg/day to about 20 μg/kg/day, about 7.5 to about 20 μg/kg/day, about 9 μg/kg/day to about 20 μg/kg/day, about 10 μg/kg/day to about 20 μg/kg/day, about 11 μg/kg/day to about 20 μg/kg/day, about 12 μg/kg/day to about 20 μg/kg/day, about 13 μg/kg/day to about 20 μg/kg/day, about 14 μg/kg/day to about 15 μg/kg/day, about 15 μg/kg/day to about 20 μg/kg/day, about 10 μg/kg/day to about 15 μg/kg/day, about 11 μg/kg/day to about 15 μg/kg/day, about 12 μg/kg/day to about 15 μg/kg/day, about 13 μg/kg/day to about 15 μg/kg/day, about 14 μg/kg/day to about 15 μg/kg/day, about 16 μg/kg/day to about 20 μg/kg/day, about 17 μg/kg/day to about 20 μg/kg/day, about 18 μg/kg/day to about 20 μg/kg/day, about 0.01 μg/kg/day, about 0.1 μg/kg/day, about 1 μg/kg/day, about 2 μg/kg/day, about 3 μg/kg/day, about 4 μg/kg/day, about 5 μg/kg/day, about 6 μg/kg/day, about 7 μg/kg/day, about 8 μg/kg/day, about 9 μg/kg/day, about 10 μg/kg/day, about 11 μg/kg/day, about 12 μg/kg/day, about 13 μg/kg/day, about 14 μg/kg/day, about 15 μg/kg/day, about 6 μg/kg/day, about 17 μg/kg/day, about 18 μg/kg/day, about 19 μg/kg/day, or about 20 μg/kg/day, of the encapsulated cells.
79. The method of any one of embodiments 43-78, wherein the pharmaceutical composition is implanted according to a method or using a device as provided for herein.
80. The method of any one of embodiments 43-79, wherein the IL-2 molecule is a native human IL-2 or an IL-2 mutein.
81. The method of any one of embodiments 43-80, wherein the heterologous oligonucleotide encoding the native human IL-2 comprises a sequence of SEQ ID NO: 1.
82. The method of any one of embodiments 43-81, wherein the heterologous oligonucleotide encoding the native human IL-2 comprises a sequence that is codon-optimized.
83. The population of encapsulated cells of embodiment 82, wherein the codon-optimized oligonucleotide encoding native human IL-2 comprises a sequence having at least 90%, 91%, 92%, 93%, 94%, 95%, 96%, 97%, 98%, 99%, or 100% sequence identity to SEQ ID NO: 3.
84. The method of any one of embodiments 43-83, wherein the cell produces recombinant native human IL-2 protein.
85. The method of any one of embodiments 43-84, wherein the recombinant native human IL-2 protein expressed by the cells comprises the amino acid sequence of: SEQ ID NO: 2.
86. The method of embodiment 85, wherein the IL-2 mutein comprises an amino acid sequence having at least 75%, 80%, 85%, 90%, 91%, 92%, 93%, 94%, 95%, 96%, 97%, 98%, or 99%, provided that the IL-2 mutein has at least one amino acid substitution as compared to SEQ ID NO: 2.
87. The method of any one of embodiments 43-86, wherein the pharmaceutical composition produces about 1 to about 10, about 1 to about 5, or about 2 to about 4 PCD (picograms/cell/day) of native human IL-2 or the IL-2 mutein.
88. The method of any one of embodiments 43-87, wherein the encapsulated cells comprise a cell as provided for herein.
89. The method of any one of embodiments 43-88, wherein the encapsulated cells comprise ARPE-19 cells comprising the heterologous oligonucleotide molecule.
90. The method of any one of embodiments 83-89, wherein the encapsulated cells are encapsulated with a polymeric hydrogel.
91. The method of embodiment 90, wherein the polymeric hydrogel comprises chitosan, cellulose, hyaluronic acid, or alginate.
92. The method of embodiments 90 or 91, wherein the polymeric hydrogel comprises alginate.
93. The method of any one of embodiments 90-92, wherein the alginate comprises SLG20.
94. The method of embodiment 93, wherein the SLG20 is about 0.1%-3% SLG20.
95. The method of any one of embodiments 43-94, wherein the cells remain viable for at least 15, 20, 25, or 28 days.
96. The method of any one of embodiments 43-95, wherein the encapsulated cells do not proliferate.
97. The method of any one of embodiments 43-96, wherein the encapsulated
cells produce a sustained amount of IL-2 for at least 5, 10, 15, 20, or 24 hours. 98. The method of any one of embodiments 43-97, wherein the encapsulated cells can produce a sustained amount of IL-2 for up to 30 days.

### H. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain alike or similar result without departing from the spirit and scope of the disclosure.

### Example 1—Intrapleural Administration of Encapsulated Cells

Six rats were given an intrapleural administration of RPE cells expressing human IL-2. The study comprised groups A and B. Group A received an administration of 20 capsules, while group B received an administration of 80 capsules. After 24 hours or 30 days following administration, presence of human IL-2 in blood (plasma) and intrapleural fluid was assayed using ELISA. The data showed no substantially detectable human IL-2 in the blood after 24 hours or 30 days of administration. Human IL-2 was found in the pleural fluid at concentrations of approximately 3000-5000 μg/ml for group A, and approximately 11,000-20,000 μg/ml for group B after 24 hours of administration. Analysis of pleural fluid at 30 days following administration showed no substantially detectable levels of human IL-2.

### Example 2—Administration of Encapsulated Cells Suppresses Tumor Growth in a Mouse Model of Mesothelioma

Female mice (BALB/C) were injected with 1×10⁵ AB1-Fluc cells in the intraperitoneal space. AB1 is a cell line is derived from mice exposed to IP injections of asbestos, commonly used to model mesothelioma. At 7 days following implantation, mice were stratified into 2 treatment groups (sham IP surgery and intraperitoneally implanted RPE mIL-2-treated) and imaged every 7 days for tumor size. The data collected at 0 days following implantation showed stable and uniform total flux among sham and mIL-2-treated animals. The data collected at 7- and 14-days following implantation showed no substantially detectable total flux in the RPE-mIL-2-treated animals as compared to sham. The data indicates that RPE-mIL-2-treatment results in tumor suppression in a mouse model of mesothelioma.

### Example 3—Administration of Encapsulated Cells Reduces Tumor Size in a Mouse Model of Mesothelioma

Mice (n=40, BALB/C) were injected with 1×10⁵ AB1-Fluc cells in the intraperitoneal space at day −7. At 7 days following administration, mice were stratified into 5 treatment groups based on total flux calculated using IVIS. The groups comprised sham, 2 capsules RPE-mIL2 capsules, 10 RPE-mIL2 capsules, 25 RPE-mIL2 capsules, and 50 RPE-mIL2 capsules, all administered intraperitoneally. The data collected at 0 days and weekly following implantation showed stable and uniform total flux among all groups. The data collected at 6 days following implantation showed dose dependent decrease in total flux, indicative of reduced size or no tumor in animals treated with RPE-mIL2 capsules.

### Example 4—Methods

Study Design: A cell-based immunotherapy was developed for local delivery of IL-2 using immunostimulatory alginate-based microparticles. Using this platform, protection of the therapeutic cells from the foreign body response was shown for extended periods of time allowing for successful reduction of MPM tumor burden without systemic toxicity. Without wishing to be bound to a particular theory, this approach amplifies therapeutic efficacy of anti PD-1, resulting in the complete eradication of local tumor, an effect not seen with anti PD-1 monotherapy.

Cell Culture and Engineering: Cell culture media and associated reagents were purchased through Fisher Scientific. Lipofection reagents (lipofectamine 3000) and selection media (puromycin) were purchased from Invitrogen. Expression vectors and helper plasmids were designed and purchased through VectorBuilder. Live Dead stains (Fisher Scientific) were used to determine cell viability of encapsulated cells. All cell lines tested negative for mycoplasma contamination. These cells were cultured using Dulbecco's Modified Eagle Medium (DMEM/F-12), with 10% Fetal Bovine Serum (FBS) and 1% antibiotic-antimycotic (AA). The media was changed 3 times weekly. Media used for AB1 cells was RPMI 1640, 10% FBS, and 1% antibiotic-antimycotic (AA).

Cell Transfection/Transduction: ARPE-19 cells (ATCC) were engineered to express cytokines of interest. AB1 cells (Sigma-Aldrich) were engineered to express firefly luciferase. Cells were transfected or transduced.

Core Shell Cell Encapsulation: Capsules were generated as described herein. Briefly, alginate was dissolved at 1.4% w/v in saline and sterile filtered. Cells were resuspended in alginate at a concentration of 40×10⁶ cells/mL. Encapsulation occurred using a custom-built, two-fluid co-axial electrostatic spraying device. Alginate droplets were expelled from a co-axial needle into barium chloride crosslinking solution where they formed hydrogel capsules. Capsules were subsequently washed with HEPES buffer and maintained with normal cell culture techniques.

Cell viability post encapsulation: Following encapsulation, a subset of capsules were washed with 5 mL DPBS and stained using a stock 2 M calcein AM and 4 M EthD-1 in DPBS. The sample was incubated for 20 minutes and imaged using a fluorescence microscope.

Enzyme-Linked Immunosorbent Assay (ELISA): An individual capsule was added to a 96 well plate (n=5-8) in 200 μl for 24 hours at 37° C. in a 5% CO2 humidified atmosphere. Cell supernatant was collected from each well and assayed via ELISA according to manufacturer protocols. Kits were obtained commercially for mouse IL-2 (R&D Systems) and human IL2 (R&D Systems). All samples were run in triplicate.

CyTOF experiments: Single cells were stabilized for 6 hours in media at 37° C. Five hundred thousand cells were resuspended in Maxpar Cell Staining Buffer (Fluidigm, Cat.No. 201068) in individual 5 mL tubes for each sample to be barcoded. Mass-tag cellular barcoding using the Cell-ID 20-Plex Pd Barcoding Kit (Fluidigm, Cat.No. 201060) was performed. Cell-ID™ Intercalator-Ir is a cationic nucleic acid intercalator that contains naturally abundant Iridium (191Ir and 193Ir) and is used for identifying nucleated cells in CyTOF analysis according to standard protocol. For measurement of intracellular cytokines by CyTOF, cells harvested from mice were incubated in 11/mL Golgistop (BD cell analysis, Cat.No.BD B554724A) for 10 hours at 37° C., according to standard protocol. The samples were then washed and incubated with cell surface antibodies for 45 minutes on ice and washed. After overnight at 4° C. with resuspension in 1×Fix I buffer, the samples were stained with intracellular antibodies against cells cytokines for 30 minutes at RT and washed. Stained cells were analyzed on a mass cytometer (CyTOF3™ mass cytometer, Fluidigm) at an event rate of 400 to 500 cells per second. All mass cytometry files were normalized together using the mass cytometry data normalization algorithm, which uses the intensity values of a sliding window of these bead standards to correct for instrument fluctuations over time and between samples. Barcodes were deconvoluted using the Debarcoder® software (Fluidigm®).

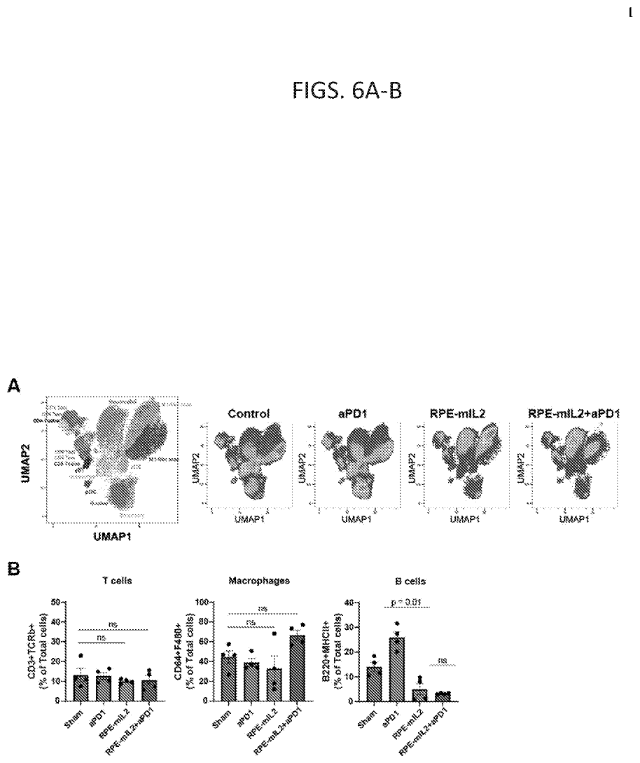

CyTOF analysis: Total live nucleated cells were used for all analyses and visualized using the Uniform Manifold Approximation and Projection (UMAP) for dimensional reduction. 40,000 immune cells were downsampled from each sample, and they were integrated into one file. Acquired single-cell data were transferred into additional cytometric analysis in FlowJo□V10 software (FlowJo, LLC, OR). To characterize all cells obtained from peritoneal lavage fluids, all cells, were organized in 14 phenotypes. Fourteen cellular phenotypes were manually defined by a panel of 43 antibodies: memory B cells (CD45+CD19+B220+CD86+), naive B cells (CD45+CD19+B220+CD86−), active CD4 T cells (CD45+CD3+TCR-β+CD4+CD44+CD69+CD62L−), effector memory CD4 T cells (CD45+CD3+TCR-β+CD4+CD44+CD69−CD62L−), naive CD4 T cells (CD45+CD3+TCR-β+CD4+CD44−CD69−CD62L+), active CD8 T cells (CD45+CD3+TCR-β+CD8+CD44+CD69+CD62L−), effector memory CD8 T cells (CD45+CD3+TCR-β+CD8+CD44+CD69−CD62L−), naive CD8 T cells (CD45+CD3+TCR-β+CD8+CD44-CD69−CD62L+), γδT cells (CD45+CD3+TCR-β−), M1-like macrophages (CD45+CD3−CD64+F4/80+CD86+PD-L1+), M2-like macrophages (CD45+CD3−CD64+F4/80+CD86−PD-L1−), plasmacytoid dendritic cells (CD45+CD3−CD19−CD11c+B220+CD317+), conventional DC (CD45+CD3−CD19−CD11c+B220−MHCII+). Mapping of our data onto its interface enabled visualization and precise quantification of immune cells in any sample as a UMAP plot, and generation of separate maps for defined groups of mice enables comparison of cellular networks between these groups. To improve efficiency and ease of display of our multiple proposed experiments, we generated the intuitive single-cell maps for each comparison (as shown in FIGS. 6A-B). Cell frequencies or proportions were compared across groups of interest. Based on the outcome of interest, statistically significant changes in cell frequencies for each cluster were shown in a single map with the directionality of change given by color. Mean metal intensities (MMI) of proteins were used to evaluate the expression of cytokines and immunoregulatory proteins.

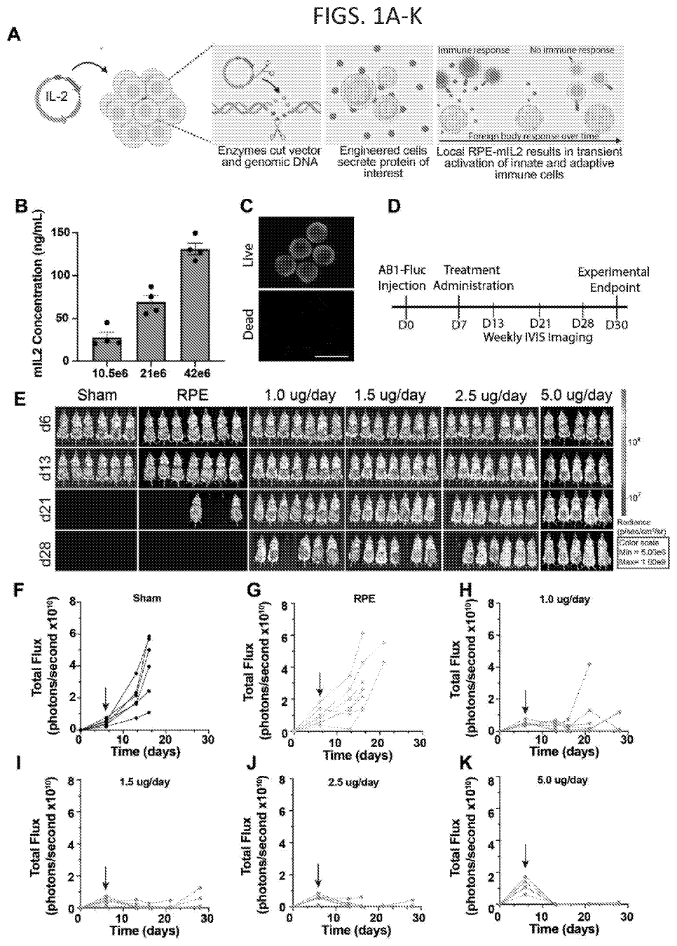

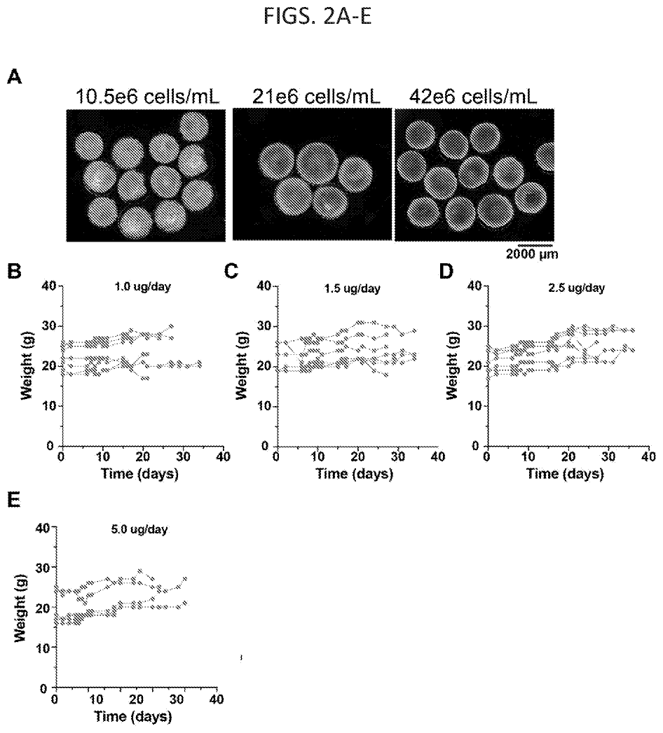

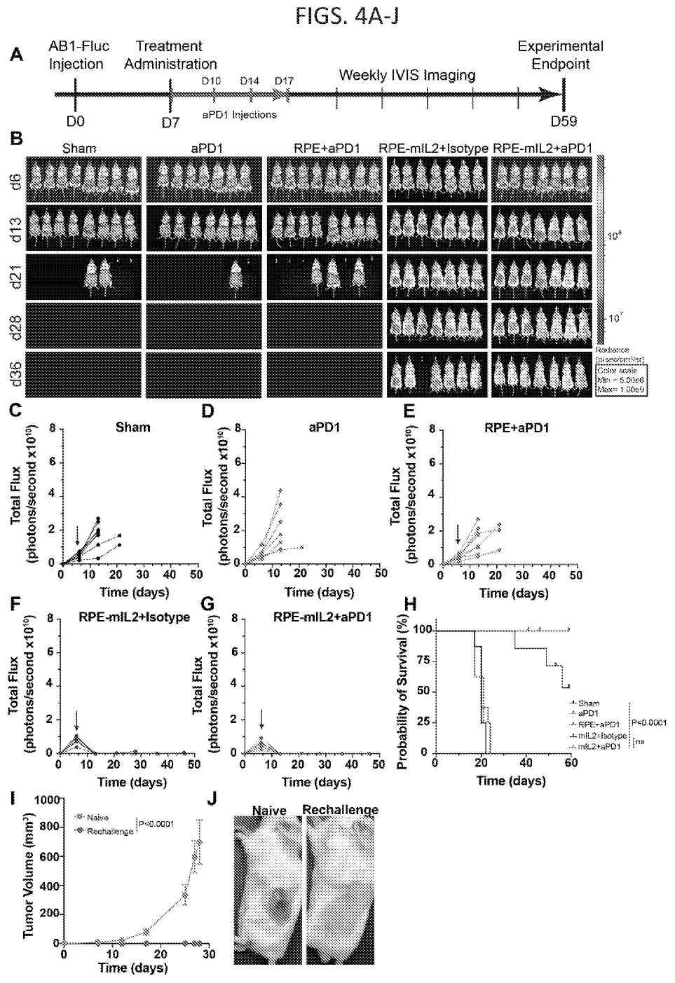

Animal Studies. Mouse Studies: Balb/C mice (Jackson Labs or Charles River Laboratories), a mixture of males and females, aged 8-10 weeks, were used for in vivo studies. All animal experiments were approved by Rice University's Institution Animal Care and Use Committee (IACUC). All biological samples implanted into animals were approved by Rice University's Institutional Biosafety Committee (IBC). For IP tumor models of AL1-Fluc; 5×10⁵ cells suspended in HBSS were intraperitoneally injected to the lower right abdomen. Tumors were injected and allowed to develop in vivo for 1 week before treatment (FIGS. 1E-K, 4B-G, FIGS. 2A-B). For all studies using IVIS imaging for tumor growth tracking, mice were imaged and stratified into treatment groups 1 day prior to surgery using the methods described in IVIS imaging section below (FIG. 1E, FIG. 4B, FIG. 2A). After stratification for tumor size, animals were randomly assigned to treatment groups. For tumor measurements, experiments were not blinded. Anti-tumor efficacy of therapy was confirmed by multiple investigators.

Experimental controls: Sham: all mice given sham surgery received IP surgery and were administered 1 mL sterile saline. RPE: RPE capsules contained the same density of cells as experimental capsules but contained naïve cells. Anti-PD1: all mice treated with a PD-1 antibodies (J43, BioXcell) received intraperitoneal injection of 200 μg per mouse at day 0, 3, 7, and 10 post treatment.

For rechallenge experiments, 5×10⁵ cells suspended in HBSS were injected subcutaneously into the rear flank of Balb/C mice that showed complete remission from intraperitoneal tumor inoculations (FIGS. 4I-J). Subcutaneous tumor growth tracking: For subcutaneous AB1 rechallenge models, the tumor size was measured using a digital caliper and tumor volume was calculated using the formula V=0.5*(height)*(width²). For immune cell depletion studies (FIGS. 2A-B), isotype control (LTF-2), anti-CD8a (2.43), or anti-CD4 (GK1.5) antibodies (BioXcell) were administered via intraperitoneal injection at a dose of 100 μg per animal at day −2, 0, and 2 post RPE-mIL2 implantation.

Intraperitoneal tumor growth tracking: Animals injected with AB1-Fluc cells were imaged using IVIS 6 days after injection and stratified into experimental groups based on luminescent signal. After surgery, animals were tracked for tumor growth or reduction using IVIS imaging 1× per week. Imaging methods are expanded below.

Intraperitoneal (IP) surgical implantation of capsules in Mice: Mice were sedated and anaesthetized. A surgical blade (15T; Sklar) was then used to cut a 0.5-0.75 cm midline incision through the skin and the linea alba into the abdomen. Capsule implants were administered using sterile transfer pipets. The abdominal muscle was closed by suturing with 5-0 Ethicon black PDS-absorbable or other 5.0-6.0 monofilament absorbable sutures. The external skin layer was closed with PDS suture as previously described.

IVIS imaging: Mice were anaesthetized and injected in the IP space with D-luciferin (300 μg/mL, 200 μL; PerkinElmer). Animals were then transferred to the IVIS manifold (IVIS Spectrum, PerkinElmer) where they were kept under isoflurane anesthesia (0.25 L/min) and maintained warm on a heated stage. Photographs and luminescent images were acquired 10 minutes after injection. Luminescent exposures were set to 1 second with the binning set at medium, the excitation set to block, the EM gain set to ‘off’ with 0-second delays between acquisitions.

H&E Staining of Explanted Organs and Capsules: Post retrieval, extracted organs or freely floating spheres were rinsed three times with PBS and fixed in 10% formalin overnight. After fixation, the spheres were rinsed twice with PBS, and dehydrated in gradually ascending ethanol solutions for 20 minutes each time. The spheres were cleared in xylene for 10 minutes, and incubated in a 50/50 solution of xylene and paraffin overnight at 57° C. On day 3, the spheres were transferred to paraffin twice for 1 hour each, and then embedded in a paraffin mould. Subsequently, embedded spheres were sectioned at 5-μm thickness onto positively charged lysine microscope slides. Tissue sections were then stained for H&E to assess pericapsular cellular overgrowth.

Rat Studies: Sprague Dawley rats were anesthetized with inhalational isoflurane in 100% 02 (5.0% induction; 2.5% maintenance). Endotracheal intubation was performed, and the animals ventilated with positive-pressure ventilation. A left lateral thoracotomy was performed. Capsules were deposited directly into the pleural cavity via Pasteur pipette and a total transfer volume of 300 ul. Each animal received one dose of 65 capsules. The chest was then sutured closed in layers and the animals were extubated and allowed to recover.

Toxicity analyses: At the scientific endpoint, rats were anesthetized and 2 mL of blood was collected from the inferior vena cava prior to euthanasia. Samples were submitted to the Mouse Metabolism and Phenotyping Core at Baylor College of Medicine. Both a Diabetes & Lipid panel, as well as a Liver panel were acquired.

Histology: At the scientific endpoint, rat hearts were perfused with PBS and excised. Lungs, liver, kidneys, and spleen were also excised. Each of the organs was fixed in 10% formalin. Formalin was exchanged for 70% ethanol after 24 hours. Organs were submitted to the Pathology Core and Lab where 5 μm tissue sections were cut and H&E stained at 0, 300, and 600 μm deep into each tissue.

Statistics: Sample size was predetermined from pilot experiments and/or experiments that have been done in the past, to obtain statistically significant data. Experiments were repeated at least once, or data were compiled from two independent experiments unless otherwise stated in the respective figure legend. Replicates were reproducible. All statistical analyses were conducted using GraphPad Prism 9. One-way ANOVA tests with the Holm-Sidak multiple comparisons methods were used to determine p values for cyTOF datasets. One-way ANOVA tests with the Holm-Sidak multiple comparisons methods were used to determine p values for toxicity assays. Unless otherwise indicated as a replicate measurement, data were taken from distinct samples.

### Example 5—IL-2-Based Cytokine Factories Result in Dose Dependent Regression of AB1 Tumors in Mice

The IL-2-based delivery system consisted of polymer encapsulated human retinal pigmented epithelial (aRPE) cells that were engineered to stably express human or mouse IL-2 (FIG. 1A) using the PiggyBAC transposon system. These xenogeneic engineered cells were then protected from the host immune system via hydrogel microencapsulation (FIG. 1A). Following encapsulation, the IL-2-based cytokine factories are referred to as RPE-mIL2 (mouse IL-2) or RPE-hIL2 (human IL-2). A delivery system with two levels of dose modulation was designed for precise IL-2 dosing and immune cell activation. First, the administered IL-2 concentration was altered by changing the density of engineered cells suspended in each capsule. Second, the dose was fine-tuned by changing the number of individual capsules in a given dose. IL-2-based cytokine factories were fabricated at four different cell densities and assayed individual capsules from each dose group for IL-2 production. The number of capsules in each dose was varied and evaluated the dose-dependent anti-tumor response in mice with AB1 tumors. The results demonstrate that as the cell density per capsule increases (FIG. 2A), the concentration of IL-2 from an individual capsule also increases (FIG. 1B) without reducing cell viability within the capsules (FIG. 1C), providing precise dose-dependent control.

To evaluate whether anti-tumor efficacy was also dose-dependent, an intraperitoneal (IP) mouse model of mesothelioma was developed and administered various doses of RPE-mIL2 according to the experimental timeline seen in FIG. 1D. The results show that increasing the number of capsules in each dose provided a dose dependent anti-tumor effect in mice bearing AB1 tumors (FIG. 1E). Tumor regression was not seen in the control animals at any time (FIGS. 1F-G). After one week of RPE-mIL2 treatment tumor reduction was greater than 45% percent, regardless of the dose, in 19/26 mice when compared to the total flux before treatment (FIGS. 1H-K). Notably, 11/12 mice treated with at least 2.5 μg of RPE-mIL2 had greater than 75% reduction in tumor burden in one week and 100% of mice treated with 5 μg of RPE-mIL2 had 90% reduction in tumor burden. Mice in the sham and capsule control (RPE) groups experienced progressive tumor growth over time (FIGS. 1F-G) while mice treated with RPE-mIL2 experienced tumor regression and extended survival (FIGS. 1H-K). Further, 17/19 mice treated with at least 1.5 μg of RPE-mIL2 survived more than 2× longer than mice in the sham group. No significant deviations in body weight over time in any of the treatment groups were observed suggesting that the therapy was well tolerated (FIGS. 2B-E). These results highlight the significant anti-tumor effects of RPE-mIL2 treatment in mice with AB1 tumors.

### Example 6—CD8+ Cytotoxic T Cells are Required for RPE-mIL2-Based Anti-Tumor Responses Seen in AB1 Tumor-Bearing Mice

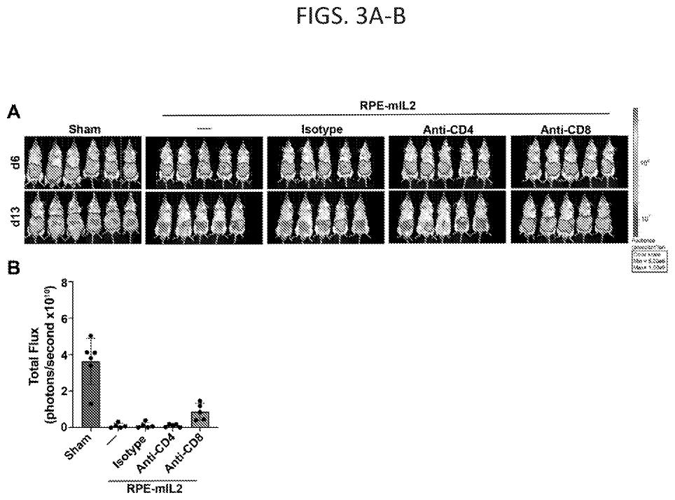

To elucidate whether CD8+ or CD4+ T cell populations (or both) were required to reproduce the tumor reduction seen in our earlier studies, antibodies against CD8+ or CD4+ T cells in AB1 tumor bearing mice treated with RPE-mIL2 were utilized. Mice lacking CD8+ T cells were unable to mount a sufficient anti-tumor response after treatment. The average total flux from this group was comparable to mice in the sham and RPE control groups (FIGS. 3A-B). The CD4+ T cell-depleted mice showed an anti-tumor response which was similar to the immune-competent mice after RPE-mIL2 treatment, suggesting that CD4+ T cells are not required to mount an anti-tumor response with our treatment (FIGS. 3A-B). Taken together, these data provide mechanistic insight into the immune cells responsible for anti-tumor efficacy after RPE-mIL2 treatment and suggest that our results are largely CD8+ T cell-dependent.

### Example 7—RPE-mIL2 in Combination with Anti-PD1 Checkpoint Therapy Eradicates AB1 Tumor Burden and Provides Protection Against Recurrence in Mice

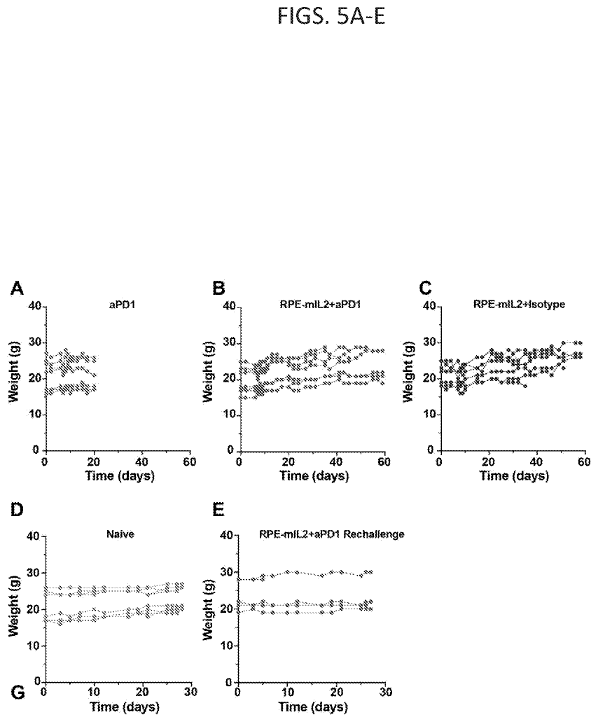

To evaluate the potential of RPE-mIL2 to increase the efficacy of checkpoint inhibitors a combination study with RPE-mIL2 and anti-PD1 (aPD1) treatment was conducted. The experiment was carried out according to the schematic seen in FIG. 4A. AB1 tumors in the intraperitoneal space of the 7/7 mice treated with RPE-mIL2+aPD1 were eradicated after one week of treatment and did not recur throughout the duration of the study (FIG. 4B). In addition, 7/7 mice treated with RPE-mIL2+isotype experienced significant reduction of tumor burden early on and tumors in 5/7 of these mice were eradicated. Mice treated with sham surgical control, PD-1 only, or RPE+aPD1 did not experience tumor regression at any time during the study and 100% of these control mice reached humane endpoints for euthanasia within three weeks after tumor administration (FIGS. 4B-G). The total flux of each animal in this study was plotted over time. Mice in each of the control groups experienced increases in total flux until they reached humane endpoints and were euthanized (FIGS. 4B-E). Mice treated with RPE-mIL2+isotype or RPE-mIL2+aPD1 never experienced a total flux higher than the starting value suggesting strong anti-tumor efficacy after RPE-mIL2 treatment (FIGS. 4B, 4F-G). Mice treated with RPE-mIL2 or RPE-mIL2+aPD1 survived significantly longer than mice in the control groups (FIG. 4H). Deviations in body weight overtime in any of the treatment groups were not observed suggesting that the therapy was well tolerated (FIGS. 5A-C). These results highlight the ability of RPE-mIL2 to act as a monotherapy and to boost the effectiveness of anti-PD1 checkpoint therapy when administered in combination.

A subset of the RPE-mIL2+aPD1 treated animals were evaluated for protection against recurrence in a rechallenge experiment. Briefly, animals treated with RPE+aPD1 were challenged with a subcutaneous injection approximately 60 days after the initial intraperitoneal administration. 100% of previously treated mice were protected from recurrence and thus did not develop subcutaneous tumors while 5/6 control mice developed large tumors with evidence of necrosis (FIGS. 4I-J) within the first 30 days. In addition, we did not observe any significant deviations in body weight in the rechallenged mice (FIG. 5E). These results suggest that this treatment may provide immunologic memory against AB1 tumors which allows for protection against recurrence.

### Example 8—RPE-mIL2 and aPD1 Combination Treatment Increased CD4+ and CD8+ T Cell Activation and Caused a Phenotypic Shift in Macrophages from M2-Like to M1-Like

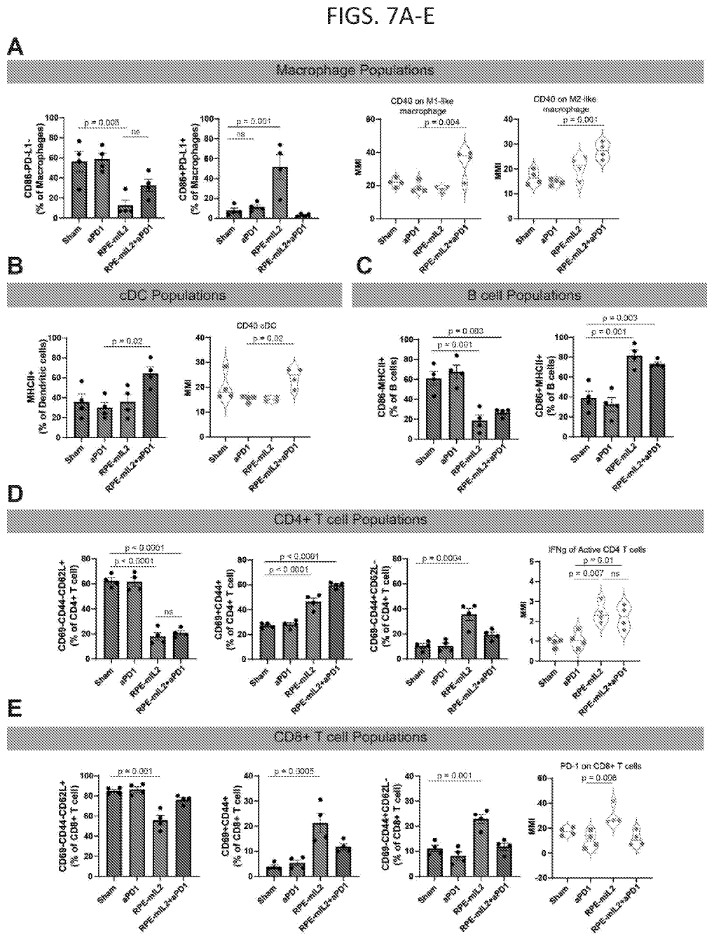

Without wishing to be bound to a particular theory, TL-2 signaling pathway is associated with immunotherapy-responsive tumors. CyTOF analysis was utilized to evaluate the changes in the presence and activation of various immune cells seven days after treatment. Briefly, mice were stratified into 4 groups and treated with sham surgery, aPD1 injection, RPE-mIL2 only, or RPE-mIL2+aPD1. Uniform Manifold Approximation and Projection (UMAP) dimension reduction was used to visualize the cellular landscape of the intraperitoneal space after treatment (FIG. 6A). The data show that there was at least 4.4× fewer intraperitoneal B cells in mice treated with either RPE-mIL2 or RPE-mIL2+aPD1 when compared to sham treated mice (FIG. 6B). A decrease in the percentage of M2-like macrophages (CD86−PD-L1−) after either RPE-mIL2 or RPE-mIL2+aPD1 combination treatment was observed (FIG. 7A). A corresponding increase in M1-like macrophages (CD86+PD-L1+) only in the mice treated with RPE-mIL2 was observed (FIG. 7A) while the combination treated mice displayed a corresponding increase in conventional dendritic cells (cDC) (MHC II+) (FIG. 7B). Further, combination treatment resulted in significantly higher levels of CD40 from both macrophages and dendritic cells which further highlights the potential of RPE-mIL2 treatment to induce immunological changes (FIGS. 7A-B).

In addition to activating the adaptive immune system, RPE-mIL2 and RPE-mIL2+aPD1 treated mice had 2.3× fewer naïve B cells and 1.9× more memory B cells than sham or aPD1 treated mice suggesting that RPE-mIL2 has a significant effect on B cell maturation (FIG. 7C). Changes in T cell subpopulations after administration of TL-2 cytokine factories were also observed. Specifically, RPE-mIL2 and RPE-mIL2+aPD1 treated mice had significantly fewer naïve (CD69−CD44−CD62L+) CD4+ and CD8+ T cells as well as increased activated (CD69+CD44+) CD4+ and CD8+ T cells when compared to sham treated mice (FIGS. 7D-E). RPE-mIL2 and RPE-mIL-2+aPD1 caused 2× higher expression of pro-inflammatory IFN-7 from activated CD4+ T cells when compared to sham or aPD1 treatment (FIG. 7D). Taken together, these data suggest that combination therapy may boost the anti-tumor potential of both CD4+ and CD8+ T cells. Further, RPE-mIL2 treatment caused a significant increase in CD4+ and CD8+ effector T cells (CD69−CD44+CD62L−) when compared to sham mice (FIGS. 7D-E) suggesting that our treatment was able to induce differentiation of critical T cell subsets in mice with mesothelioma. Finally, a 1.7× increase of PD-1 on CD8+ T cells was observed from mice treated with RPE-mIL2 when compared to the sham group. This suggests a potential rationale for the success seen when used in combination with anti-PD1 therapy (FIG. 7D). Taken together, these results suggest that RPE-mIL2 treatment has the potential to activate both innate and adaptive immune cells when administered in mice with AB1 tumors.

### Example 9—RPE-hIL2 can be Safely Administered to the Intraperitoneal or Pleural Cavity and is Well-Tolerated in Mice and Rats

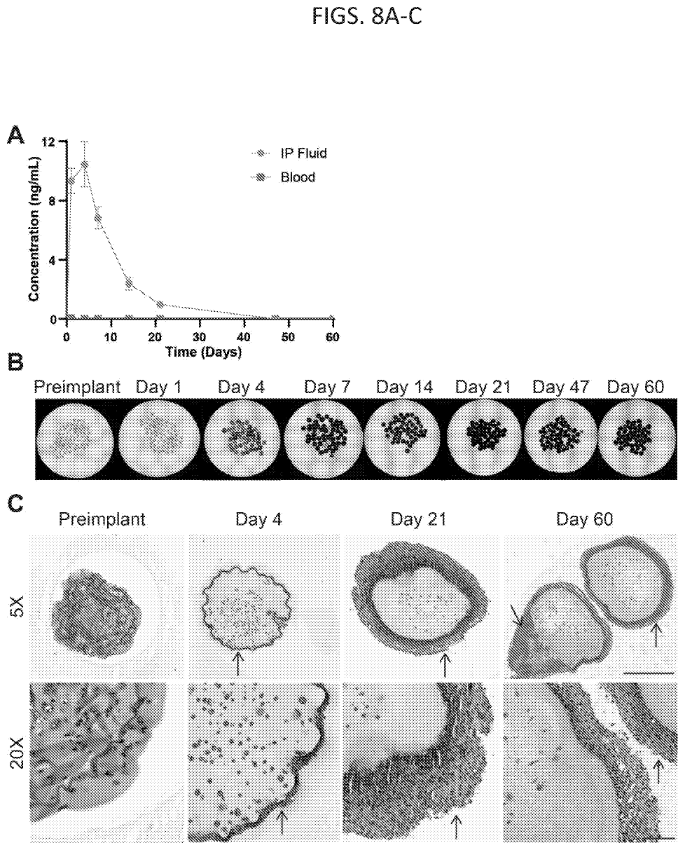

To address feasibility of dosing and the reproducibility of the foreign body response, the in vivo pharmacokinetics of RPE-hIL2 in the intraperitoneal (IP) space of immunocompetent mice was evaluated. The local (IP fluid) hIL2 concentration peaked by day 4 after implantation and declined at a rate inversely proportional to the pericapsular fibrotic overgrowth (PFO) accumulation on the surface of the capsules (FIGS. 8A-B) suggesting that PFO accumulation plays a role in RPE-hIL2 treatment duration. To assess the extent of fibrotic overgrowth and the stage/activity of the FBR for the RPE-hIL2 platform, the PFO at select time points (day 0, 4, 21, and 60) was evaluated using H&E staining to assess the extent of fibrotic overgrowth (FIG. 8C). By day 60, a thick coating could clearly be seen encompassing the capsule(s), but the host cells on the surface of the capsules no longer possessed distinct membranes and nuclei, thus demonstrating that the cells were not viable, the FBR remodeling was complete and the capsule-PFO particles were inert. All animals tolerated both the cell-delivered hIL2 and the alginate microcapsules at all-time points. This data was key for ensuring that the capsules: 1) did not continue to deliver cytokines after treatment completion and 2) did not pose a safety issue to the patients at extended periods.

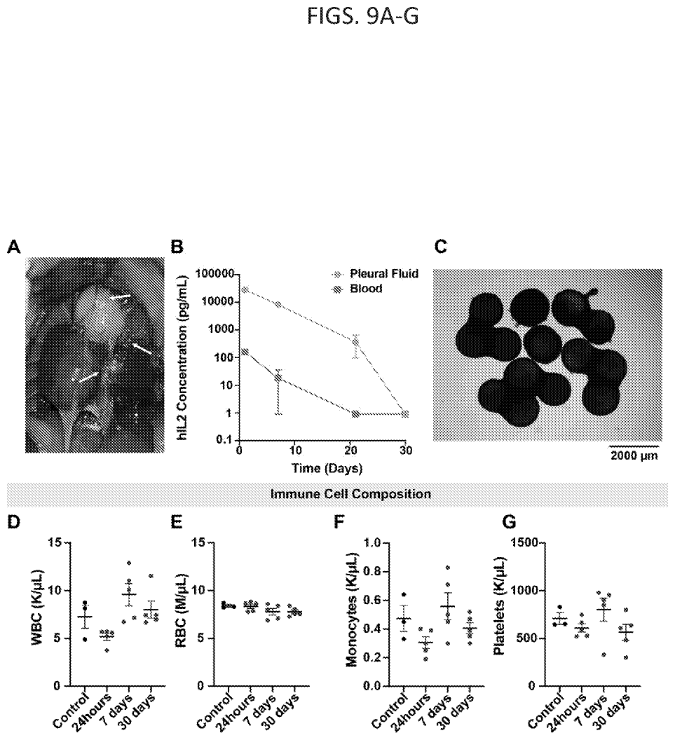

To study the safety and translatability of hIL2 administration in the pleural cavity, the effects of RPE-hIL2 (2 μg/day) in the pleural cavity of Sprague Dawley rats was evaluated. RPE-hIL2 cytokine factories were successfully administered to the pleural cavity in 20/20 rats (FIG. 9A) which highlights the feasibility of administration to this cavity. The hIL2 concentration peaked 24 hours after administration in the pleural fluid and the blood (FIG. 9B) and that the local concentration was at least 100× greater than the systemic concentration at all time points. Similar to the results seen in mice, the cytokine factories were heavily coated with pericapsular overgrowth by day 30 post-treatment (FIG. 9C). Any significant deviations from control values in WBC, RBC, monocyte, or platelet concentrations at any time during our study were not observed. These data suggest that the cytokine factories were well tolerated by the host immune system.

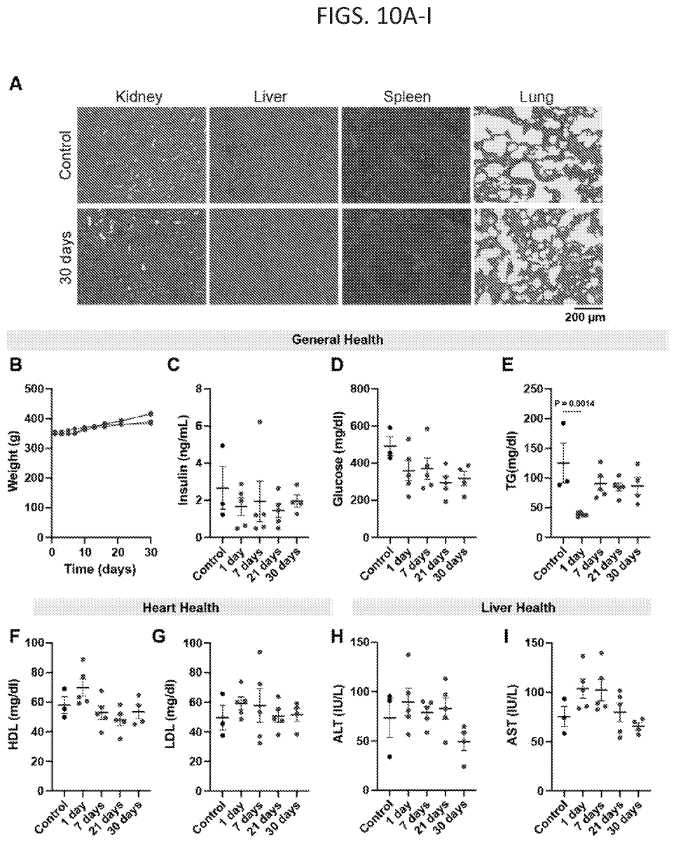

The liver, kidney, and lungs are often implicated in IL2-related toxicities so we used H&E staining to assess the histopathologic condition of these organs 30 days after RPE-hIL2 administration. No major histopathological changes in cells of the kidney, liver, spleen, or lungs were observed when compared to control animals at the conclusion of our study (FIG. 10A). In addition, no significant changes in body weight, insulin levels, or glucose levels was observed suggesting that the treatment was well tolerated (FIGS. 10B-D). A decrease in triglyceride levels 24 hours after administration was recorded, but this drop was transient and was managed by the animals without any intervention (FIG. 10E). Finally, no significant changes in HDL (FIG. 10F), LDL (FIG. 10G), ALT (FIG. 10H), or AST (FIG. 10I) levels were observed when compared to control animals which suggest healthy heart and liver function. In total, 20/20 rats dosed with RPE-hIL2 managed the cytokine factories without complication. To further highlight the translational potential of this platform, successful administration of cytokine factories was demonstrated via intrapleural catheters in porcine cadavers. Taken together, these data suggest that RPE-hIL2 can be safely and successfully administered to the pleural cavity and this work as a whole provides a rationale for translation into clinical studies for patients with pleural or intraperitoneal malignant mesothelioma.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

1. A method of treating a mesothelioma, in a subject, the method comprising implanting, or delivering to, the pleural cavity a pharmaceutical composition comprising a population of encapsulated cells comprising a heterologous oligonucleotide molecule encoding the native human IL-2.

2. The method of claim 1, wherein the mesothelioma is a pleural mesothelioma, a malignant pleural mesothelioma, or a diffuse pleural mesothelioma.

3. A method of treating a mesothelioma, in a subject by generating memory immunity, the method comprising implanting, or delivering to, the pleural cavity a pharmaceutical composition comprising a population of encapsulated cells comprising a heterologous oligonucleotide molecule encoding the native human IL-2.

4. The method of claim 3, wherein the mesothelioma is a pleural mesothelioma, a malignant pleural mesothelioma, or a diffuse pleural mesothelioma.

5. A method of delivering a native IL-2 to the pleural cavity of subject, the method comprising implanting, or delivering to, the pleural cavity a pharmaceutical composition comprising a population of encapsulated cells comprising a heterologous oligonucleotide molecule encoding the native human IL-2.

6. A method of treating a disease, in a subject, the method comprising implanting, or delivering to, the pleural cavity of the subject a pharmaceutical composition comprising a population of encapsulated cells comprising a heterologous oligonucleotide molecule encoding the native human IL-2.

7. The method of claim 2, wherein the disease is as provided herein.

8. A method of treating a pleural disease or condition, in a subject, the method comprising implanting, or delivering to, the pleural cavity a pharmaceutical composition comprising a population of encapsulated cells comprising a heterologous oligonucleotide molecule encoding the native human IL-2.

9. A method of treating a pleural disease or condition, in a subject by generating memory immunity, the method comprising implanting, or delivering to, the pleural cavity a pharmaceutical composition comprising a population of encapsulated cells comprising a heterologous oligonucleotide molecule encoding the native human IL-2.

10. The method of claim 8, wherein the pleural disease or condition is pleural cancer, pleural metastatic disease, pleurisy, lung infection, viral pneumonia, bacterial pneumonia, idiopathic pulmonary fibrosis, acute respiratory distress syndrome, pleural thickening, pleural pseudotumor, pleural plaque, extrapleural hematoma, Castleman disease, hemangioendothelioma, splenosis, paramalignang effusion, pleural effusion, pneumothorax, hemothorax, reactive pleuritis.

11. The method of claim 10, wherein the pleural cancer is mesothelioma, lung cancer, metastases, malignant mesothelioma, lymphoma, malignant fibrous tumor, sarcoma, askin tumor, extraskeletal osteosarcoma, malignant fibrous histiocytoma, solitary fibrous tumor, lipoma, mesothelial cyst, calcifying fibrous pseudotumor, primary effusion lymphoma.

12. A method of providing systemic treatment to a subject with cancer, the method comprising implanting in the pleural cavity of the subject a pharmaceutical composition comprising a population of encapsulated cells comprising a heterologous oligonucleotide molecule encoding the native human IL-2, whereby the pharmaceutical composition stimulates the activation of immune cells in the pleural cavity and the activated immune cells migrate to a region of the subject that is distal to the pleural cavity to treat the cancer systemically in the subject.

13. A method of providing systemic treatment to a subject with cancer, the method comprising implanting in the pleural cavity of the subject a pharmaceutical composition comprising a population of encapsulated cells comprising a heterologous oligonucleotide molecule encoding the native human IL-2, whereby the pharmaceutical composition activates immune cells and the activated immune cells migrate out of the pleural cavity to treat the cancer in the subject.

14. The method of claim 12, wherein the subject has fewer side effects as compared to a subject that is administered the pharmaceutical composition systemically, such as intravenously.

15. The method of claim 12, wherein the activated immune cells are CD8 positive effector T cells.

16. The method of claim 12, wherein the effector T cells are selectively activated and expanded at least 1, 2, 3, 4, or 5 times as compared to Tregs in the pleural cavity.

17. The method of claim 12, wherein the effector T cells are selectively activated and expanded at least 1, 2, 3, 4, or 5 times as compared to Tregs systemically.

18. The method of claim 1, wherein the oligonucleotide encoding native human IL-2 comprises a sequence of SEQ ID NO: 1:

19. The method of claim 1, wherein the oligonucleotide encoding native human IL-2 comprises a sequence that is codon-optimized, wherein the codon-optimized oligonucleotide encoding native human IL-2 comprises a sequence having at least 90%, 91%, 92%, 93%, 94%, 95%, 96%, 97%, 98%, 99%, or 100% sequence identity to SEQ ID NO: 3.

20. The method of claim 1, wherein the recombinant native human IL-2 protein expressed by the cells comprises the amino acid sequence of: SEQ ID NO: 2.

21. The method of claim 1, wherein the encapsulated cells are ARPE-19 cells comprising the heterologous oligonucleotide molecule.

22. The method of claim 1, wherein the encapsulated cells are encapsulated with a polymeric hydrogel, such as chitosan, cellulose, hyaluronic acid, or alginate.

23. The method of claim 21, wherein the polymeric hydrogel comprises alginate, such as SLG20.

24. The method of claim 1, further comprising administering an additional therapeutic.

25. The method of claim 24, wherein the additional therapeutic is an immunomodulatory agent.

26. The method of claim 25, wherein the immunomodulatory agent is an inhibitor of PD-1, PD-L1, PD-L2, CTLA4, TIM3, LAG3, VISTA, BTLA, TIGIT, LAIR1, CD73, CD160, 2B4 and/or TGFRβ.

27. The method of claim 26, wherein the inhibitor is an anti-PD-1 antibody, anti-PD-L1 antibody, anti-PD-L2 antibody, anti-CTLA4 antibody, anti-TIM3 antibody, anti-LAG3 antibody, anti-VISTA antibody, anti-BTLA antibody, anti-TIGIT antibody, anti-LAIR1 antibody, anti-CD73 antibody, anti-CD160 antibody, anti-2B4 antibody, anti-TGFRβ antibody, or any combination thereof.

28. The method of claim 27, wherein the anti-PD-1 antibody is selected from pembrolizumab, nivolumab, cemiplimab, atezolizumab, dostralimab, durvalumab, avelumab, or any combination thereof.

29. A method of delivering a native cytokine and an therapeutic to the subject, the method comprising implanting, or delivering to, the a pharmaceutical composition comprising a population of encapsulated cells a heterologous oligonucleotide molecule encoding the native human cytokine and further administering a pharmaceutical composition comprising an additional therapeutic.

30. A method of treating a disease or condition, in a subject, the method comprising implanting, or delivering to, the subject a pharmaceutical composition comprising a population of encapsulated cells comprising a heterologous oligonucleotide molecule encoding an IL-2 molecule and further administering a pharmaceutical composition comprising an additional therapeutic.

31. The method of claim 30, wherein the disease or condition is a cancer, such as mesothelioma.

32. The method of claim 31, wherein the mesothelioma is a pleural mesothelioma, peritoneal mesothelioma, pericardial mesothelioma, testicular mesothelioma, epithelioid mesothelioma, sarcomatoid mesothelioma, biphasic mesothelioma, small cell mesothelioma, deciduoid mesothelioma, cystic and papillary mesothelioma, desmoplastic mesothelioma, adenomatoid mesothelioma, heterologous mesothelioma, well-defined papillary cell mesothelioma, or any combination thereof.

33. A method of treating mesothelioma in a subject, the method comprising implanting, or delivering to, the subject a pharmaceutical composition comprising a population of encapsulated cells comprising a heterologous oligonucleotide molecule encoding the native human cytokine and further administering a pharmaceutical composition comprising an additional therapeutic.

34. The method of claim 29, wherein the cytokine is IL-2, IL-12, IL-1, IL-1α, IL-1β, IL-1RA, IL-4, IL-5, IL-6, IL-7, IL-8, IL-9, IL-10, IL-11, IL-12a, IL-12b, IL-13, IL-14, IL-15, IL-16, IL-17, G-CSF, GM-CSF, IL-20, IL-23, IFN-α, IFN-β, IFN-γ, CD154, LT-β, CD70, CD153, CD178, TRAIL, TNF-α, TNF-β, SCF, M-CSF, MSP, 4-1BBL, LIF, OSM, or any combination thereof.

35. The method of claim 29, wherein the additional therapeutic is an immunomodulatory agent.

36. The method of claim 35, wherein the immunomodulatory agent is an inhibitor of PD-1, PD-L1, PD-L2, CTLA4, TIM3, LAG3, VISTA, BTLA, TIGIT, LAIR1, CD73, CD160, 2B4 and/or TGFRβ.

37. The method of claim 36, wherein the inhibitor is an anti-PD-1 antibody, anti-PD-L1 antibody, anti-PD-L2 antibody, anti-CTLA4 antibody, anti-TIM3 antibody, anti-LAG3 antibody, anti-VISTA antibody, anti-BTLA antibody, anti-TIGIT antibody, anti-LAIR1 antibody, anti-CD73 antibody, anti-CD160 antibody, anti-2B4 antibody, anti-TGFRβ antibody, or any combination thereof.

38. The method of claim 37, wherein the anti-PD-1 antibody is selected from pembrolizumab, nivolumab, cemiplimab, atezolizumab, dostralimab, durvalumab, avelumab, or any combination thereof.

39. A method of treating mesothelioma in a subject, the method comprising implanting, or delivering to, the subject a pharmaceutical composition comprising a population of encapsulated cells comprising a heterologous oligonucleotide molecule encoding an IL-2 molecule and further administering a pharmaceutical composition comprising an immunomodulatory agent.

40. The method of claim 39, wherein the treatment results in activation or increase of immune cells.

41. The method of claim 40, wherein the activated immune cells are CD4 and CD8 positive T cells.

42. The method of claim 40, wherein the increased immune cells are CD4 and CD8 positive effector T cells.

43. The method of claim 29, wherein the treatment results in macrophage phenotype shift, such as from M2-like macrophages to M1-like macrophages.

44. The method of claim 39, wherein the treatment results in increase in MHC II+ dendritic cells.

45. A method of providing systemic treatment to a subject with cancer, the method comprising
implanting in a cavity of the subject a pharmaceutical composition comprising a population of encapsulated cells comprising a heterologous oligonucleotide molecule encoding an IL-2 molecule; and
administering an immunomodulatory agent;
whereby the pharmaceutical composition stimulates the activation of immune cells in the cavity and the activated immune cells migrate to a region of the subject that is distal to the cavity to treat the cancer systemically in the subject.

46. The method of claim 45, wherein the cancer is mesothelioma.

47. A method of providing systemic treatment to a subject with cancer, the method comprising
implanting in a cavity of the subject a pharmaceutical composition comprising a population of encapsulated cells comprising a heterologous oligonucleotide molecule encoding an IL-2 molecule; and
administering an immunomodulatory agent;
whereby the pharmaceutical composition activates immune cells and the activated immune cells migrate out of the cavity to treat the cancer in the subject.

48. The method of claim 47, wherein the cancer is mesothelioma.
